(19) United States
(12) Patent Application Publication
(10) Pub. No.: US 2026/0191837 A1
(43) Pub. Date: Jul. 9, 2026

(54) BET INHIBITORS FOR THE TREATMENT OF CANCER

(71) Applicant: Leapfrog Bio, Inc., South San Francisco, CA (US)

(72) Inventors: Tomas BABAK, Oakland, CA (US); Peter TRUESDELL, Kingston (CA)

(21) Appl. No.: 19/557,803

(22) Filed: Mar. 5, 2026

Related U.S. Application Data
(63) Continuation of application No. PCT/US2024/045429, filed on Sep. 5, 2024.
(60) Provisional application No. 63/581,241, filed on Sep. 7, 2023.
(60) Provisional application No. 63/625,852, filed on Jan. 26, 2024.

(51) Int. Cl. A61K 31/4365; A61K 31/437; A61K 31/444; A61K 31/472; A61K 31/4985; A61K 31/5025; A61K 31/517; A61K 31/519; A61K 31/5383; A61K 31/55; A61K 31/5517; A61K 38/17; A61P 35/00

(52) U.S. Cl. CPC A61K 31/4365; A61K 31/437; A61K 31/444; A61K 31/472; A61K 31/4985; A61K 31/5025; A61K 31/517; A61K 31/519; A61K 31/5383; A61K 31/55; A61K 31/5517; A61K 38/1709; A61P 35/00

(57) ABSTRACT

Provided herein are methods of using a BET inhibitor for the treatment of a cancer comprising a loss of function or a deletion of an EP300 gene or portion thereof.

### CROSS REFERENCE

This application is a continuation of International Application No. PCT/US2024/045429, filed Sep. 5, 2024, which claims the benefit of U.S. Provisional Application No. 63/581,241, filed Sep. 7, 2023, and U.S. Provisional Application No. 63/625,852, filed Jan. 26, 2024, the contents of each being hereby incorporated herein by reference.

### BACKGROUND

BET inhibitors are a class of drugs that target a family of proteins called Bromodomain and Extra-Terminal (BET) proteins. These proteins may play a key role in regulating gene expression and chromatin structure, cell cycle progression, and other important cell biology functions. Their involvement in cell growth and differentiation makes them potentially important to the pathophysiology of cancer. Among a number of distinct mechanisms of action, BET inhibitors have the ability to disrupt the interaction between BET proteins and chromatin, leading to changes in gene expression that can inhibit cancer cell growth and survival. Although they have shown promise in a few studies, lack of clinical efficacy, dose-limiting toxicities and the prevalence of resistance to BET inhibitors remain a significant challenge. Accordingly, there is a need for the development of novel BET inhibitor treatments for cancer-targeted therapies.

### BRIEF SUMMARY OF THE INVENTION

The present disclosure is based, in part, on the discovery that cancers may be associated with a loss of function or a deletion of an EP300 gene, and once identified, such cancers may be treated by administering (e.g., a therapeutically effective amount) a pharmaceutical composition comprising a Bromodomain and Extra-Terminal (BET) inhibitor.

Provided herein, in some embodiments, are methods of treating a cancer in a subject in need thereof, said method comprising administering a pharmaceutical composition to said subject, wherein said pharmaceutical composition comprises a bromodomain and extra-terminal domain (BET) inhibitor; said cancer has previously been determined to comprise a loss of function or a deletion of an EP300 gene; said patient is not concurrently receiving

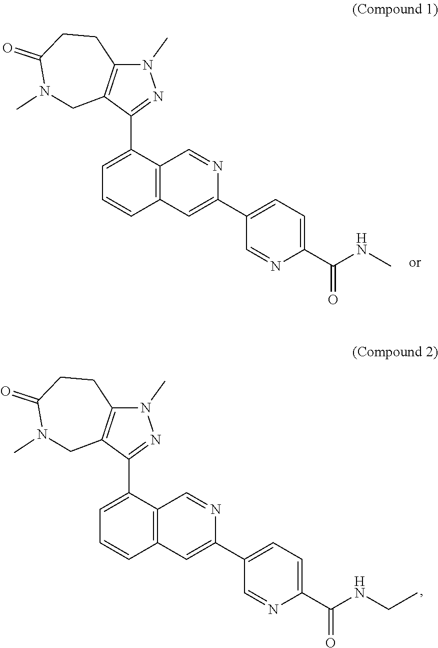 [SMILES: CNC(=O)c1ccc(-c2cc3cccc(-c4nn(C)c5c4CN(C)C(=O)CC5)c3cn2)cn1 ; CCNC(=O)c1ccc(-c2cc3cccc(-c4nn(C)c5c4CN(C)C(=O)CC5)c3cn2)cn1]

and said BET inhibitor is not JQ1 when said cancer is triple negative breast cancer.

Provided herein, in some embodiments, are methods of treating a cancer in a subject in need thereof, said method comprising administering a pharmaceutical composition to said subject, wherein said pharmaceutical composition comprises a bromodomain and extra-terminal domain (BET) inhibitor; said cancer has previously been determined to comprise a decreased amount or activity of p300 compared to wild type p300; said patient is not concurrently

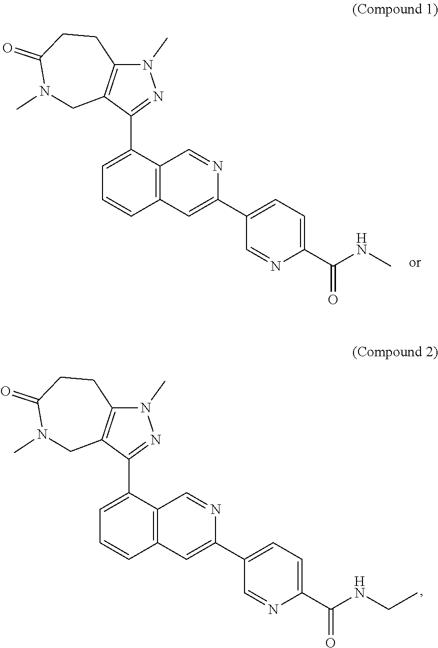 [SMILES: CNC(=O)c1ccc(-c2cc3cccc(-c4nn(C)c5c4CN(C)C(=O)CC5)c3cn2)cn1 ; CCNC(=O)c1ccc(-c2cc3cccc(-c4nn(C)c5c4CN(C)C(=O)CC5)c3cn2)cn1]

said BET inhibitor is not JQ1 when said cancer is triple negative breast cancer. In some embodiments, said loss of function is caused by genetic mutation. In some embodiments, said subject has not previously received Compound 1 or Compound 2. In some embodiments, said BET inhibitor comprises ABBV-075, ABBV-744, Apabetalone, APL-581, ARV-825, AZD-5153, BI-6727, BI-894999, BMS-986158, BOS-475, BPI-23314, CD-161, CG-223, CK-103, CN-470, FT-1101, GNE-0011, GS-5829, GS-626510, GSK525762, I-BET151, INCB054329, INCB57643, JQ1, LY-294002, NE02734, ODM-207, OMT-001, OMT-002, OTX-015, Pelabresib, PLX-2853 (OPN-2853), PLX-51107 (OPN-51107), TEN-010, RVX-297, SRX-2523, SRX-3225, SRX-3254, SW-064652, SYHA-1801, TTI-281, Zen-3694, CPI-0610, INCB0543294, INCB057643, CC-90010, Alobresib, GSK778, GSK046, Mivebresib, Trotabresib, Molibresib, Pelabresib or a combination thereof. In some embodiments, said BET inhibitor comprises AZD-5153, BI-894999, BMS-986158, GSK525762, INCB054329, INCB57643, OTX-015, PLX-51107, or a combination thereof. In some embodiments, said BET inhibitor comprises AZD-5153, BI-894999, BMS-986158, GSK525762, INCB054329, OTX-015, PLX-51107, INCB057643, TEN-010, CC-90010, Alobresib, ODM-207, GSK778, GSK046, ABBV-744, Mivebresib, Trotabresib, Molibresib, Birabresib, Pelabresib, or a combination thereof.

In some embodiments, said BET inhibitor comprises a compound of Formula (I):

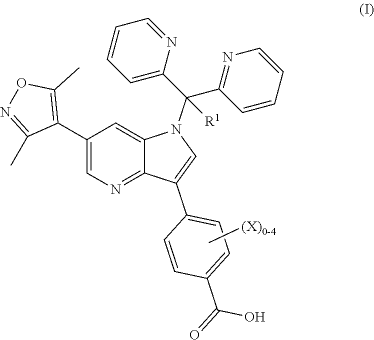

or a pharmaceutically acceptable salt, a tautomer, a stereoisomer or a deuterated analog thereof, wherein: R¹ is cyano, halo, or (C₁-C₃)alkyl optionally substituted with 1 to 3 substituents independently selected from the group consisting of halo, methyl, ethyl, methoxy and ethoxy; and X, when present, is halo. In some embodiments, R¹ is (C₁-C₂)alkyl, cyano or fluoro. In some embodiments, R¹ is methyl. In some embodiments, R¹ is fluoro. In some embodiments, R¹ is cyano.

In some embodiments, said BET inhibitor comprises a compound of Formula (II):

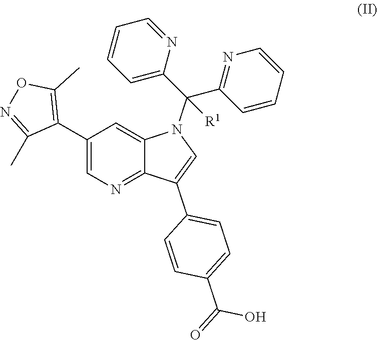

or a pharmaceutically acceptable salt, a tautomer, a stereoisomer, or a deuterated analog thereof, wherein: R¹ is (C₁-C₃)alkyl optionally substituted with 1 to 3 substituents independently selected from the group consisting of halo, methyl, ethyl, methoxy and ethoxy.

In some embodiments, R¹ is methyl.

In some embodiments, said BET inhibitor comprises a compound of Formula (III):

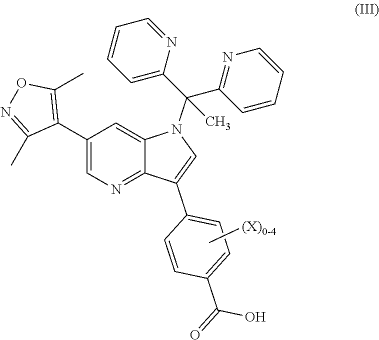

or a pharmaceutically acceptable salt thereof, wherein: X, when present, is halo.

In some embodiments, said BET inhibitor comprises Compound 3:

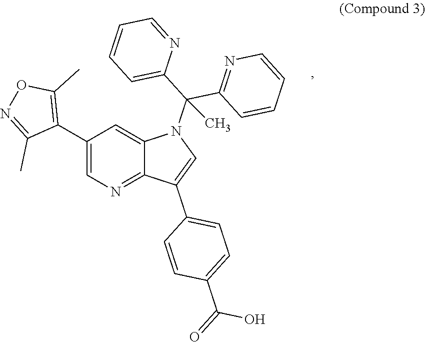 [SMILES: Cc1noc(C)c1-c1cnc2c(-c3ccc(C(=O)O)cc3)cn(C(C)(c3ccccn3)c3ccccn3)c2c1]

or a pharmaceutically acceptable salt, a tautomer, a solvate, or a deuterated analog thereof. In some embodiments, said BET inhibitor comprises a compound having formula (Va):

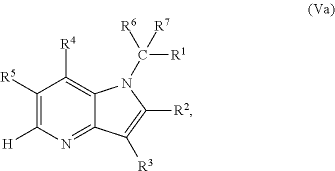

or a pharmaceutically acceptable salt, a solvate, a tautomer, a stereoisomer or a deuterated analog thereof, wherein: R² is H; R⁴ is H; R⁶ is H; R⁷ is H, —OH, C₁₋₆ alkyl, aryl, heteroaryl, cycloalkyl, or heterocycloalkyl; R¹ is heteroaryl optionally substituted with from 1-3 R^{j} groups; each R^{j} independently selected from halogen, —CN, —OH, —NH₂, —NO₂, —C(O)OH, —C(S)OH, —C(O)NH₂, —C(S)NH₂, —S(O)₂NH₂, —NHC(O)NH₂, —NHC(S)NH₂, —NHS(O)₂NH₂, —C(NH)NH₂, —CH═C(R^{k})(R^{k}), —OR^{k}, —SR^{k}, —OC(O)R^{k}, —OC(S)R^{k}, —P(═O)HR^{k}, —P(═O)R^{k}R^{k}, —PH(═O)OR^{k}, —P(═O)(OR^{k})₂, —OP(═O)(OR^{k})₂, —C(O)H, —O(CO)OR^{k}, —C(O)R^{k}, —C(S)R^{k}, —C(O)OR^{k}, —C(S)OR^{k}, —S(O)R^{k}, —S(O)₂R^{k}, —C(O)NHR^{k}, —C(S)NHR^{k}, —C(O)NR^{k}R^{k}, —C(S)NR^{k}R^{k}, —S(O)₂NHR^{k}, —S(O)₂NR^{k}R^{k}, —C(NH)NHR^{k}, —C(NH)NR^{k}R^{k}, —NHC(O)R^{k}, —NHC(S)R^{k}, —NR^{k}C(O)R^{k}, —NR^{k}C(S)R^{k}, —NHS(O)₂R^{k}, —NR^{k}S(O)₂R^{k}, —NHC(O)NHR^{k}, —NHC(S)NHR^{k}, —NR^{k}C(O)NH₂, —NR^{k}C(S)NH₂, —NR^{k}C(O)NHR^{k}, —NR^{k}C(S)NHR^{k}, —NHC(O)NR^{k}R^{k}, —NHC(S)NR^{k}R^{k}, —NR^{k}C(O)NR^{k}R^{k}, —NR^{k}C(S)NR^{k}R^{k}, —NHS(O)₂NHR^{k}, —NR^{k}S(O)₂NH₂, —NR^{k}S(O)₂NHR^{k}, —NHS(O)₂NR^{k}R^{k}, —NR^{k}S(O)₂NR^{k}R^{k}, —NHR^{k} or —NR^{k}R^{k}; each R^{k} is independently H, C₁₋₆alkyl, aryl, arylalkyl, heteroaryl, heteroarylalkyl, heterocycloalkyl, heterocycloalkylalkyl, cycloalkyl or cycloalkylalkyl; or two R^{k} groups when attached to the same carbon or nitrogen atom are taken together to form a 3- to 6-membered carbocyclic ring or 3- to 8-membered heterocyclic ring having from 1-2 heteroatoms as ring members selected from O, N or S, wherein the nitrogen or sulfur ring atoms are optionally oxidized; R³ is H, halogen, —CN, optionally substituted C₁₋₆alkyl, optionally substituted deuterated C₁₋₆alkyl, optionally substituted aryl, optionally substituted aryl-C₁₋₄alkyl, optionally substituted heteroaryl, optionally substituted heteroaryl-C₁₋₄alkyl, optionally substituted C₃₋₈ cycloalkyl, optionally substituted C₃₋₈ cycloalkyl-C₁₋₄alkyl, optionally substituted heterocycloalkyl, or optionally substituted heterocycloalkyl-C₁₋₄alkyl; and R⁵ is

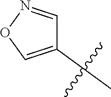 [SMILES: *c1cnoc1]

optionally substituted with from 1 to 2 R¹¹ groups independently selected from D, halogen, C₁₋₆alkyl, C₁₋₄haloalkyl, C₁₋₄haloalkoxy or —CN; wherein the wavy line indicates the point of attachment to the rest of molecule. In some embodiments, said BET inhibitor comprises Compound 4:

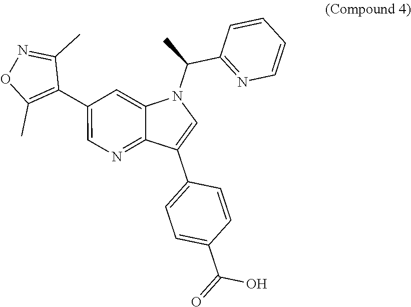 [SMILES: Cc1noc(C)c1-c1cnc2c(-c3ccc(C(=O)O)cc3)cn([C@@H](C)c3ccccn3)c2c1]

or a pharmaceutically acceptable salt, a tautomer, a solvate, or a deuterated analog thereof.

In some embodiments, said BET inhibitor comprises Compound 9:

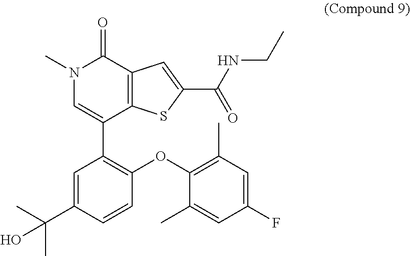 [SMILES: CCNC(=O)c1cc2c(=O)n(C)cc(-c3cc(C(C)(C)O)ccc3Oc3c(C)cc(F)cc3C)c2s1]

or a pharmaceutically acceptable salt, a tautomer, a solvate, or a deuterated analog thereof. In some embodiments, said cancer comprises bladder cancer, lung cancer, gynecologic cancer, adrenocortical carcinoma, bone cancer, central nervous system (CNS) cancer, pancreatic cancer, gastrointestinal cancer, head and neck cancer, skin cancer, mesothelioma, nerve sheath tumor, lymphoma, renal cell carcinoma, salivary gland cancer, non-melanoma skin cancer, or a combination thereof. In some embodiments, said cancer comprises lung cancer. In some embodiments, said lung cancer comprises non-small cell lung cancer. In some embodiments, said non-small cell lung cancer comprises squamous cell carcinoma, large cell carcinoma, adenocarcinoma, or a combination thereof. In some embodiments, said non-small cell lung cancer comprises squamous cell carcinoma. In some embodiments, said non-small cell lung cancer comprises large cell carcinoma. In some embodiments, said non-small cell lung cancer comprises adenocarcinoma. In some embodiments, said lung cancer comprises small cell lung cancer. In some embodiments, said BET inhibitor comprises AZD-5153, INCB57643, OTX-015, PLX-51107, or a combination thereof. In some embodiments, said cancer comprises bladder cancer. In some embodiments, said bladder cancer comprises a carcinoma. In some embodiments, said carcinoma comprises urothelial carcinoma. In some embodiments, said BET inhibitor comprises AZD-5153, BMS-986158, GSK525762, INCB57643, OTX-015, PLX-51107, or a combination thereof. In some embodiments, said cancer comprises pancreatic cancer. In some embodiments, said BET inhibitor comprises BI-894999, BMS-986158, GSK525762, INCB054329, INCB57643, OTX-015, PLX-51107, or a combination thereof. In some embodiments, said skin cancer comprises melanoma. In some embodiments, said CNS cancer comprises a glioma. In some embodiments, said CNS cancer comprises a neuroepithelial tumor. In some embodiments, said lymphoma comprises Non-Hodgkin lymphoma. In some embodiments, said lymphoma comprises diffuse large B cell lymphoma. In some embodiments, said lymphoma comprises follicular lymphoma. In some embodiments, said lymphoma comprises marginal zone lymphoma. In some embodiments, said lymphoma comprises mature B-cell neoplasm. In some embodiments, said gastrointestinal cancer comprises a gastrointestinal stromal tumor, esophagogastric cancer, gastrointestinal neuroendocrine tumor, small bowel cancer, anal cancer, colon cancer, or a combination thereof. In some embodiments, said gastrointestinal cancer comprises a gastrointestinal stromal tumor. In some embodiments, said gastrointestinal cancer comprises an esophagogastric cancer. In some embodiments, said gastrointestinal cancer comprises a gastrointestinal neuroendocrine tumor. In some embodiments, said gastrointestinal cancer comprises a small bowel cancer. In some embodiments, said gastrointestinal cancer comprises an anal cancer. In some embodiments, said gastrointestinal cancer comprises colon cancer. In some embodiments, said colon cancer comprises colorectal cancer. In some embodiments, said gynecologic cancer comprises a cervical cancer. In some embodiments, said gynecologic cancer comprises an ovarian cancer. In some embodiments, said gynecologic cancer comprises a sex cord stromal tumor. In some embodiments, said gynecologic cancer comprises a vaginal cancer. In some embodiments, said cancer comprises uterine cancer. In some embodiments, said uterine cancer comprises endometrial cancer or uterine sarcoma. In some embodiments, said uterine cancer comprises endometrial cancer. In some embodiments, said endometrial cancer comprises uterine corpus endometrial carcinoma. In some embodiments, said uterine cancer comprises uterine sarcoma. In some embodiments, said uterine sarcoma comprises uterine carcinosarcoma. In some embodiments, said BET inhibitor inhibits any one or more of the following bromodomains: BRD2, BRD3, and BRD4. In some embodiments, said BET inhibitor inhibits BRD4 and BRD2. In some embodiments, said BET inhibitor inhibits BRD3 and BRD4. In some embodiments, said BET inhibitor inhibits BD1 bromodomain of BRD2, BRD3, BRD4, or any combination thereof. In some embodiments, said BET inhibitor inhibits BD2 bromodomain of BRD2, BRD3, BRD4, or any combination thereof. In some embodiments, said BET inhibitor comprises a selective BET inhibitor. In some embodiments, said selective BET inhibitor inhibits any one or more of the following bromodomains: BRD2, BRD3, and BRD4. In some embodiments, said selective BET inhibitor is selective for BRD4 and BRD2. In some embodiments, said selective BET inhibitor is selective for BRD2, BRD3, and BRD4. In some embodiments, said selective BET inhibitor inhibits BD1 bromodomain of BRD2, BRD3, BRD4, or any combination thereof. In some embodiments, said selective BET inhibitor comprises GSK778 In some embodiments, said selective BET inhibitor inhibits BD2 bromodomain of BRD2, BRD3, BRD4, or any combination thereof. In some embodiments, said selective BET inhibitor comprises GSK046, ABBV-744, Compound 9, or any combination thereof. In some embodiments, said loss of function or deletion is for an EP300 gene.

Provided herein, in some embodiments, are methods of treating a cancer in a subject in need thereof, said method comprising administering a pharmaceutical composition to said subject, wherein said pharmaceutical composition comprises a bromodomain and extra-terminal domain (BET) inhibitor; said cancer has previously been determined to comprise a loss of function or a deletion of an EP300 gene;
wherein said BET inhibitor comprises a compound of formula IV,

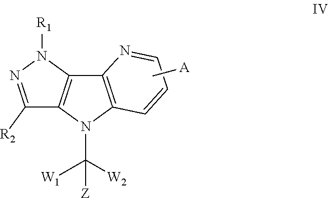

or a pharmaceutically acceptable salt thereof or a stereoisomer thereof, wherein:
R₁ is selected from hydrogen; deuterium; —C₁₋₆alkyl; or —C₃₋₈carbocyclic; and each of which at each occurrence is independently optionally substituted with 1, 2, 3, 4, 5 or 6 substituents, and the said each of substituents at each occurrence is independently selected from deuterium, halogen, OH, —CN, —C₁₋₈alkyl, —C₁₋₈alkoxy, —NH₂, —NH(C₁₋₆alkyl), —N(C₁₋₆alkyl)₂, or —C₃₋₈carbocyclic; R₂ is selected from hydrogen; deuterium; halogen; —OR₂₁; —NR₂₁R₂₂; —CN; —SR₂₁; —SOR₂₁; —SO₂R₂₁; —SO₂NR₂₁R₂₂; —C₁₋₈alkyl;

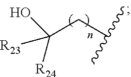

carboxyl; —COOR₂₁; —CONR₂₁R₂₂; —NR₂₁COR₂₂; —NR₂₁SO₂R₂₂; or —C₃₋₈carbocyclic; and each of which at each occurrence is independently optionally substituted with 1, 2, 3, 4, 5 or 6 substituents, and the said each of substituents at each occurrence is independently selected from deuterium, halogen, —OH, —CN, —NH₂, —C₁₋₈alkyl, —C₁₋₈alkoxy, C₃₋₈carbocyclic, or 6-membered heterocyclic containing 1, 2 or 3 heteroatoms selected from N, O; each of R₂₁ and R₂₂ at each occurrence is independently selected from hydrogen; deuterium; —OH; NH₂; —CN; —C₁₋₈alkyl; —C₁₋₈alkoxy; —C₁₋₈alkylene-C₃₋₈carbocyclic; or —C₃₋₈carbocyclic; each of R₂₃ and R₂₄ at each occurrence is independently selected from hydrogen, deuterium, or —C₁₋₈alkyl; A is selected from

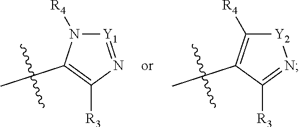

Y₁ is selected from N or CR_{Y1}; Y₂ is selected from O, S, CR_{Y1}R_{Y2} or NR_{Y2}; each of R_{Y1} and R_{Y2} at each occurrence is independently selected from hydrogen, deuterium, halogen, —OH, NH₂, —CN, —C₁₋₆alkyl or —C₁₋₆alkoxy; each of R₃ and R₄ at each occurrence is independently selected from hydrogen, deuterium, or —C₁₋₆alkyl; and each of which at each occurrence is independently optionally substituted with 1, 2, 3, 4, 5 or 6 substituents, and the said each of substituents at each occurrence is independently selected from deuterium, halogen, —OH, —NH₂, —CN, —C₁₋₆alkyl or —C₁₋₆alkoxy; n is selected from 0, 1, 2, 3, 4, 5 or 6; W₁ is selected from hydrogen; deuterium; —F; —CI; —NH₂; —CN; —OH; carboxyl; —C₁₋₆alkyl; —C₁₋₆alkoxy; —C₁₋₃alkylene-C₁₋₃alkoxy; phenyl; 5-membered heteroaryl containing 1, 2 or 3 heteroatoms selected from N, O; 6-membered heteroaryl containing 1, 2 or 3 heteroatoms selected from N, O; 3-membered heterocyclic containing 1, 2 or 3 heteroatoms selected from N, O; 4-membered heterocyclic containing 1, 2 or 3 heteroatoms selected from N, O; 5-membered heterocyclic containing 1, 2 or 3 heteroatoms selected from N, O; 6-membered heterocyclic containing 1, 2 or 3 heteroatoms selected from N, O; 3-membered carbocyclic; 4-membered carbocyclic; 5-membered carbocyclic; or 6-membered carbocyclic; and each of which at each occurrence is independently optionally substituted with 1, 2, 3, 4, 5 or 6 substituents, and the said each of substituents at each occurrence is selected from deuterium, halogen, —NH₂, —CN, —OH, —NO₂, carboxyl, —C₁₋₃alkyl, or —C₁₋₃alkoxy; W₂ is selected from hydrogen; deuterium; —F; —CI; —NH₂; —CN; —OH; carboxyl; —C₁₋₃alkyl; —C₁₋₃alkoxy; phenyl; naphthyl; 5-membered heteroaryl containing 1, 2 or 3 heteroatoms selected from N, O or S; 6-membered heteroaryl containing 1, 2 or 3 heteroatoms selected from N, O, or S; 7-membered heteroaryl containing 1, 2 or 3 heteroatoms selected from N, O or S; 8-membered heteroaryl containing 1, 2 or 3 heteroatoms selected from N, O or S; 9-membered heteroaryl containing 1, 2 or 3 heteroatoms selected from N, O or S; 10-membered heteroaryl containing 1, 2 or 3 heteroatoms selected from N, O, or S; 3-membered heterocyclic containing 1, 2 or 3 heteroatoms selected from N, O or S; 4-membered heterocyclic containing 1, 2 or 3 heteroatoms selected from N, O or S; 5-membered heterocyclic containing 1, 2 or 3 heteroatoms selected from N, O or S; 6-membered heterocyclic containing 1, 2 or 3 heteroatoms selected from N, O or S; 3-membered carbocyclic; 4-membered carbocyclic; 5-membered carbocyclic; or 6-membered carbocyclic; and each of which at each occurrence is independently optionally substituted with 1, 2, 3, 4 or 5 substituents, and the said each of substituents at each occurrence is selected from deuterium, halogen, —NH₂, —CN, —OH, —NO₂, carboxyl, methyl, ethyl, propyl, isopropyl, methoxy, ethoxy, propoxy or isopropoxy; Z is selected from hydrogen, deuterium, halogen, —NH₂, —CN, —OH, carboxyl, —C₁₋₆alkyl or —C₁₋₆alkoxy.

Provided herein, in some embodiments, are methods of treating a cancer in a subject in need thereof, said method comprising administering a pharmaceutical composition to said subject, wherein said pharmaceutical composition comprises a bromodomain and extra-terminal domain (BET) inhibitor; said cancer has previously been determined to comprise a decreased amount or activity of p300 compared to wild type p300; wherein said BET inhibitor comprises a compound of formula IV,

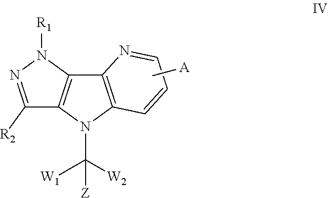

or a pharmaceutically acceptable salt thereof or a stereoisomer thereof wherein: R₁ is selected from hydrogen; deuterium; —C₁₋₆alkyl; or —C₃₋₈carbocyclic; and each of which at each occurrence is independently optionally substituted with 1, 2, 3, 4, 5 or 6 substituents, and the said each of substituents at each occurrence is independently selected from deuterium, halogen, OH, —CN, —C₁₋₈alkyl, —C₁₋₈alkoxy, —NH₂, —NH(C₁₋₆alkyl), —N(C₁₋₆alkyl)₂, or —C₃₋₈carbocyclic; R₂ is selected from hydrogen; deuterium; halogen; —OR₂₁; —NR₂₁R₂₂; —CN; —SR₂₁; —SOR₂₁; —SO₂R₂₁; —SO₂NR₂₁R₂₂; —C₁₋₈alkyl;

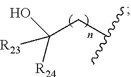

carboxyl; —COOR₂₁; —CONR₂₁R₂₂; —NR₂₁COR₂₂; —NR₂₁SO₂R₂₂; or —C₃₋₈carbocyclic; and each of which at each occurrence is independently optionally substituted with 1, 2, 3, 4, 5 or 6 substituents, and the said each of substituents at each occurrence is independently selected from deuterium, halogen, —OH, —CN, —NH₂, —C₁₋₈alkyl, —C₁₋₈alkoxy, C₃₋₈carbocyclic, or 6-membered heterocyclic containing 1, 2 or 3 heteroatoms selected from N, O; each of R₂₁ and R₂₂ at each occurrence is independently selected from hydrogen; deuterium; —OH; NH₂; —CN; —C₁₋₈alkyl; —C₁₋₈alkoxy; —C₁₋₈alkylene-C₃₋₈carbocyclic; or —C₃₋₈carbocyclic; each of R₂₃ and R₂₄ at each occurrence is independently selected from hydrogen, deuterium, or —C₁₋₈alkyl; A is selected from

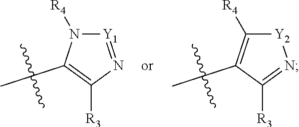

Y₁ is selected from N or CR_{Y1}; Y₂ is selected from O, S, CR_{Y1}R_{Y2} or NR_{Y2}; each of R_{Y1} and R_{Y2} at each occurrence is independently selected from hydrogen, deuterium, halogen, —OH, NH₂, —CN, —C₁₋₆alkyl or —C₁₋₆alkoxy; each of R₃ and R₄ at each occurrence is independently selected from hydrogen, deuterium, or —C₁₋₆alkyl; and each of which at each occurrence is independently optionally substituted with 1, 2, 3, 4, 5 or 6 substituents, and the said each of substituents at each occurrence is independently selected from deuterium, halogen, —OH, —NH₂, —CN, —C₁₋₆alkyl or —C₁₋₆alkoxy; n is selected from 0, 1, 2, 3, 4, 5 or 6; W₁ is selected from hydrogen; deuterium; —F; —CI; —NH₂; —CN; —OH; carboxyl; —C₁₋₆alkyl; —C₁₋₆alkoxy; —C₁₋₃alkylene-C₁₋₃alkoxy; phenyl; 5-membered heteroaryl containing 1, 2 or 3 heteroatoms selected from N, O; 6-membered heteroaryl containing 1, 2 or 3 heteroatoms selected from N, O; 3-membered heterocyclic containing 1, 2 or 3 heteroatoms selected from N, O; 4-membered heterocyclic containing 1, 2 or 3 heteroatoms selected from N, O; 5-membered heterocyclic containing 1, 2 or 3 heteroatoms selected from N, O; 6-membered heterocyclic containing 1, 2 or 3 heteroatoms selected from N, O; 3-membered carbocyclic; 4-membered carbocyclic; 5-membered carbocyclic; or 6-membered carbocyclic; and each of which at each occurrence is independently optionally substituted with 1, 2, 3, 4, 5 or 6 substituents, and the said each of substituents at each occurrence is selected from deuterium, halogen, —NH₂, —CN, —OH, —NO₂, carboxyl, —C₁₋₃alkyl, or —C₁₋₃alkoxy; W₂ is selected from hydrogen; deuterium; —F; —CI; —NH₂; —CN; —OH; carboxyl; —C₁₋₃alkyl; —C₁₋₃alkoxy; phenyl; naphthyl; 5-membered heteroaryl containing 1, 2 or 3 heteroatoms selected from N, O or S; 6-membered heteroaryl containing 1, 2 or 3 heteroatoms selected from N, O, or S; 7-membered heteroaryl containing 1, 2 or 3 heteroatoms selected from N, O or S; 8-membered heteroaryl containing 1, 2 or 3 heteroatoms selected from N, O or S; 9-membered heteroaryl containing 1, 2 or 3 heteroatoms selected from N, O or S; 10-membered heteroaryl containing 1, 2 or 3 heteroatoms selected from N, O, or S; 3-membered heterocyclic containing 1, 2 or 3 heteroatoms selected from N, O or S; 4-membered heterocyclic containing 1, 2 or 3 heteroatoms selected from N, O or S; 5-membered heterocyclic containing 1, 2 or 3 heteroatoms selected from N, O or S; 6-membered heterocyclic containing 1, 2 or 3 heteroatoms selected from N, O or S; 3-membered carbocyclic; 4-membered carbocyclic; 5-membered carbocyclic; or 6-membered carbocyclic; and each of which at each occurrence is independently optionally substituted with 1, 2, 3, 4 or 5 substituents, and the said each of substituents at each occurrence is selected from deuterium, halogen, —NH₂, —CN, —OH, —NO₂, carboxyl, methyl, ethyl, propyl, isopropyl, methoxy, ethoxy, propoxy or isopropoxy; Z is selected from hydrogen, deuterium, halogen, —NH₂, —CN, —OH, carboxyl, —C₁₋₆alkyl or —C₁₋₆alkoxy. In some embodiments, said loss of function is caused by genetic mutation. In some embodiments, said BET inhibitor comprises Compound 5:

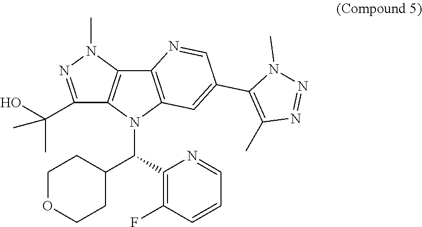 [SMILES: Cc1nnn(C)c1-c1cnc2c3c(c(C(C)(C)O)nn3C)n([C@H](c3ncccc3F)C3CCOCC3)c2c1]

or a pharmaceutically acceptable salt, a tautomer, a solvate, or a deuterated analog thereof.

In some embodiments, said BET inhibitor comprises Compound 6:

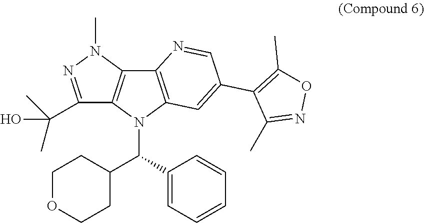 [SMILES: Cc1noc(C)c1-c1cnc2c3c(c(C(C)(C)O)nn3C)n([C@H](c3ccccc3)C3CCOCC3)c2c1]

or a pharmaceutically acceptable salt, a tautomer, a solvate, or a deuterated analog thereof.

In some embodiments, said BET inhibitor comprises Compound 7:

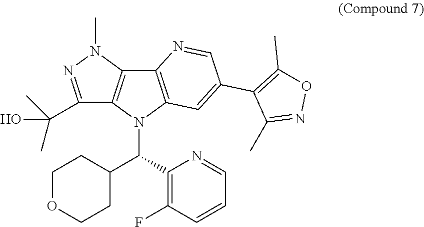 [SMILES: Cc1noc(C)c1-c1cnc2c3c(c(C(C)(C)O)nn3C)n([C@H](c3ncccc3F)C3CCOCC3)c2c1]

or a pharmaceutically acceptable salt, a tautomer, a solvate, or a deuterated analog thereof.

In some embodiments, said BET inhibitor comprises Compound 8:

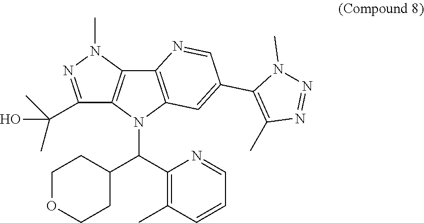 [SMILES: Cc1cccnc1C(C1CCOCC1)n1c2cc(-c3c(C)nnn3C)cnc2c2c1c(C(C)(C)O)nn2C]

or a pharmaceutically acceptable salt, a tautomer, a solvate, or a deuterated analog thereof. In some embodiments, said cancer comprises bladder cancer, lung cancer, gynecologic cancer, adrenocortical carcinoma, bone cancer, central nervous system (CNS) cancer, pancreatic cancer, gastrointestinal cancer, head and neck cancer, skin cancer, mesothelioma, nerve sheath tumor, lymphoma, renal cell carcinoma, salivary gland cancer, non-melanoma skin cancer, or a combination thereof. In some embodiments, said cancer comprises lung cancer. In some embodiments, said lung cancer comprises non-small cell lung cancer. In some embodiments, said non-small cell lung cancer comprises squamous cell carcinoma, large cell carcinoma, adenocarcinoma, or a combination thereof. In some embodiments, said non-small cell lung cancer comprises squamous cell carcinoma. In some embodiments, said non-small cell lung cancer comprises large cell carcinoma. In some embodiments, said non-small cell lung cancer comprises adenocarcinoma. In some embodiments, said lung cancer comprises small cell lung cancer. In some embodiments, said cancer comprises bladder cancer. In some embodiments, said bladder cancer comprises a carcinoma. In some embodiments, carcinoma comprises urothelial carcinoma. In some embodiments, said cancer comprises pancreatic cancer. In some embodiments, said skin cancer comprises melanoma. In some embodiments, said CNS cancer comprises a glioma. In some embodiments, said CNS cancer comprises a neuroepithelial tumor. In some embodiments, said lymphoma comprises Non-Hodgkin lymphoma. In some embodiments, said lymphoma comprises diffuse large B cell lymphoma. In some embodiments, said lymphoma comprises follicular lymphoma. In some embodiments, said lymphoma comprises marginal zone lymphoma. In some embodiments, said lymphoma comprises mature B-cell neoplasm. In some embodiments, said gastrointestinal cancer comprises a gastrointestinal stromal tumor, esophagogastric cancer, gastrointestinal neuroendocrine tumor, small bowel cancer, anal cancer, colon cancer, or a combination thereof. In some embodiments, said gastrointestinal cancer comprises a gastrointestinal stromal tumor. In some embodiments, said gastrointestinal cancer comprises an esophagogastric cancer. In some embodiments, said gastrointestinal cancer comprises a gastrointestinal neuroendocrine tumor. In some embodiments, said gastrointestinal cancer comprises a small bowel cancer. In some embodiments, said gastrointestinal cancer comprises an anal cancer. In some embodiments, said gastrointestinal cancer comprises colon cancer. In some embodiments, said colon cancer comprises colorectal cancer. In some embodiments, said gynecologic cancer comprises a cervical cancer. In some embodiments, said gynecologic cancer comprises an ovarian cancer. In some embodiments, said gynecologic cancer comprises a sex cord stromal tumor. In some embodiments, said gynecologic cancer comprises a vaginal cancer. In some embodiments, said cancer comprises uterine cancer. In some embodiments, said uterine cancer comprises endometrial cancer or uterine sarcoma. In some embodiments, said uterine cancer comprises endometrial cancer. In some embodiments, said endometrial cancer comprises uterine corpus endometrial carcinoma. In some embodiments, said uterine cancer comprises uterine sarcoma. In some embodiments, said uterine sarcoma comprises uterine carcinosarcoma. In some embodiments, said subject has a NUT midline cancer. In some embodiments, said subject has a castration resistant prostate cancer. In some embodiments, said subject has a solid cancer or a liquid cancer. In some embodiments, said subject has a liquid cancer selected from myelofibrosis. In some embodiments, said subject has a liquid cancer selected from myeloma and leukemia. In some embodiments, said subject has a liquid cancer selected from leukemia. In some embodiments, the leukemia is selected from acute myeloid leukemia (AMIL) and acute lymphocytic leukemia (ALL). In some embodiments, said loss of function or deletion is for an EP300 gene. In some embodiments, R₁ is selected from hydrogen; deuterium; —C₁₋₆alkyl; or —C₃₋₈carbocyclic; and each of which at each occurrence is independently optionally substituted with 1, 2, 3, 4, 5 or 6 substituents, and the said each of substituents at each occurrence is independently selected from deuterium, halogen, OH, —CN, —C₁₋₈alkyl, or —C₁₋₈alkoxy; R₂ is selected from hydrogen; deuterium; halogen; —C₁₋₈alkyl;

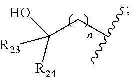

carboxyl; —COOR₂₁; or —CONR₂₁R₂₂; and each of which at each occurrence is independently optionally substituted with 1, 2, 3, 4, 5 or 6 substituents, and the said each of substituents at each occurrence is independently selected from deuterium, halogen, —OH, —CN, —NH₂, —C₁₋₈alkyl, —C₁₋₈alkoxy, C₃₋₈carbocyclic, or 6-membered heterocyclic containing 1, 2 or 3 heteroatoms selected from N, O; each of R₂₁ and R₂₂ at each occurrence is independently selected from hydrogen; deuterium; —OH; NH₂; —CN; —C₁₋₈alkyl; or —C₃₋₈carbocyclic; each of R₂₃ and R₂₄ at each occurrence is independently selected from hydrogen, deuterium, or —C₁₋₈alkyl; A is selected from

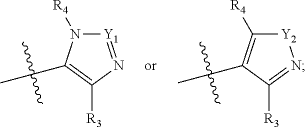

Y₁ is selected from N or CR_{Y1}; Y₂ is selected from O, S, CR_{Y1}R_{Y2} or NR_{Y2}; each of R_{Y1} and R_{Y2} at each occurrence is independently selected from hydrogen, deuterium, halogen, —OH, NH₂, —CN, —C₁₋₆alkyl or —C₁₋₆alkoxy; each of R₃ and R₄ at each occurrence is independently selected from hydrogen, deuterium, or —C₁₋₆alkyl; and each of which at each occurrence is independently optionally substituted with 1, 2, 3, 4, 5 or 6 substituents, and the said each of substituents at each occurrence is independently selected from deuterium, halogen, —OH, —NH₂, —CN, —C₁₋₆alkyl or —C₁₋₆alkoxy; n is selected from 0, 1 or 2; W₁ is selected from hydrogen; deuterium; —F; —CI; —NH₂; —CN; —OH; carboxyl; —C₁₋₆alkyl; —C₁₋₆alkoxy; 6-membered heterocyclic containing 1, 2 or 3 heteroatoms selected from N, O; and each of which at each occurrence is independently optionally substituted with 1, 2, 3, 4, 5 or 6 substituents, and the said each of substituents at each occurrence is selected from deuterium, halogen, —NH₂, —CN, —OH, —NO₂, carboxyl, —C₁₋₃alkyl, or —C₁₋₃alkoxy; W₂ is selected from hydrogen; deuterium; —F; —CI; —NH₂; —CN; —OH; carboxyl; —C₁₋₃alkyl; —C₁₋₃alkoxy; phenyl; naphthyl; 5-membered heteroaryl containing 1, 2 or 3 heteroatoms selected from N, O or S; 6-membered heteroaryl containing 1, 2 or 3 heteroatoms selected from N, O, or S; and each of which at each occurrence is independently optionally substituted with 1, 2, 3, 4 or 5 substituents, and the said each of substituents at each occurrence is selected from deuterium, halogen, —NH₂, —CN, —OH, —NO₂, carboxyl, methyl, ethyl, propyl, isopropyl, methoxy, ethoxy, propoxy or isopropoxy; Z is selected from hydrogen, deuterium, halogen, —NH₂, —CN, —OH, or —C₁₋₆alkoxy. In some embodiments, said compound of formula IV is selected from: (S)-2-(6-(3,5-dimethylisoxazol-4-yl)-1-methyl-4-(phenyl(tetrahydro-2H-pyran-4-yl)methyl)-1,4-dihydropyrazolo[3′,4′:4,5]pyrrolo[3,2-b]pyridin-3-yl)propan-2-ol; (S)-2-(6-(3,5-dimethylisoxazol-4-yl)-4-((3-fluoropyridin-2-yl)(tetrahydro-2H-pyran-4-yl)methyl)-1-methyl-1,4-dihydropyrazolo[3′,4′:4,5]pyrrolo[3,2-b]pyridin-3-yl)propan-2-ol; 2-(6-(1,4-Dimethyl-1H-1,2,3-triazol-5-yl)-1-methyl-4-(4,4,4-trifluoro-1-(3-fluoropyridin-2-yl)butyl)-1,4-dihydropyrazolo[3′,4′:4,5]pyrrolo[3,2-b]pyridine-3-yl)propan-2-ol; 2-(6-(1,4-Dimethyl-1H-1,2,3-triazol-5-yl)-1-methyl-4-((3-methylpyridin-2-yl)(tetrahydro-2H-pyran-4-yl)methyl)-1,4-dihydropyrazolo[3′,4′:4,5]pyrrolo[3,2-b]pyridine-3-yl)propan-2-ol; (S)-2-(6-(1,4-dimethyl-1H-1,2,3-triazol-5-yl)-1-methyl-4-(phenyl(tetrahydro-2H-pyran-4-yl)methyl)-1,4-dihydropyrazolo[3′,4′:4,5]pyrrolo[3,2-b]pyridine-3-yl)propan-2-ol; (S)-2-(6-(1,4-dimethyl-1H-1,2,3-triazol-5-yl)-4-((3-fluoropyridin-2-yl)(tetrahydro-2H-pyran-4-yl)methyl)-1-methyl-1,4-dihydropyrazolo[3′,4′:4,5]pyrrolo[3,2-b]pyridin-3-yl)propan-2-ol; 2-(6-(3,5-dimethylisoxazol-4-yl)-1-methyl-4-((tetrahydro-2H-pyran-4-yl)(o-tolyl)methyl)-1,4-dihydropyrazolo[3′,4′:4,5]pyrrolo[3,2-b]pyridin-3-yl)propan-2-ol; (S)-2-(6-(1,4-Dimethyl-1H-1,2,3-triazol-5-yl)-4-((3-fluoropyridin-2-yl)(tetrahydro-2H-pyran-4-yl)methyl)-1,4-dihydropyrazolo[3′,4′:4,5]pyrrolo[3,2-b]pyridin-3-yl)propan-2-ol; 6-(1,4-Dimethyl-1H-1,2,3-triazol-5-yl)-1-methyl-4-(phenyl(tetrahydro-2H-pyran-4-yl)methyl)-1,4-dihydropyrazolo[3′,4′:4,5]pyrrolo[3,2-b]pyridine-3-carboxamide; 2-(6-(1,4-Dimethyl-1H-1,2,3-triazol-5-yl)-1-methyl-4-(phenyl(tetrahydro-2H-pyran-4-yl)methyl)-1,4-dihydropyrazolo[3′,4′:4,5]pyrrolo[3,2-b]pyridine-3-yl)propan-2-amine; 2-(4-((3-Fluoropyridin-2-yl)(tetrahydro-2H-pyran-4-yl)methyl)-1-methyl-6-(1-methyl-4-(methyl-d3)-1H-1,2,3-triazol-5-yl)-1,4-dihydropyrazolo[3′,4′:4,5]pyrrolo[3,2-b]pyridine-3-yl)propan-2-ol; 2-(6-(1,4-Dimethyl-1H-1,2,3-triazol-5-yl)-4-((3-fluoropyridin-4-yl)(tetrahydro-2H-pyran-4-yl)methyl)-1-methyl-1,4-dihydropyrazolo[3′,4′:4,5]pyrrolo[3,2-b]pyridine-3-yl)propan-2-ol; 2-(6-(1,4-Dimethyl-1H-1,2,3-triazol-5-yl)-4-((3-methoxypyridin-2-yl)(tetrahydro-2H-pyran-4-yl)methyl)-1-methyl-1,4-dihydropyrazolo[3′,4′:4,5]pyrrolo[3,2-b]pyridin-3-yl)propan-2-ol; and 4-((6-(1,4-dimethyl-1H-1,2,3-triazol-5-yl)-1-methyl-4-(phenyl(tetrahydro-2H-pyran-4-yl)methyl)-1,4-dihydropyrazolo[3′,4′:4,5]pyrrolo[3,2-b]pyridin-3-yl)methyl)morpholine.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A demonstrates the inhibitive activity of AZD-5153 in H2009 cells with either wildtype EP300 or with EP300 knocked out.

FIG. 1B demonstrates the inhibitive activity of AZD-5153 in SW780 cells with either wildtype EP300 or with EP300 knocked out.

FIG. 2A demonstrates the inhibitive activity of BI-2536 in SW780 cells with either wildtype EP300 or with EP300 knocked out.

FIG. 2B demonstrates the inhibitive activity of BI-2536 in KP4 cells with either wildtype EP300 or with EP300 knocked out.

FIG. 3 demonstrates the inhibitive activity of BI-894999 in KP4 cells with either wildtype EP300 or with EP300 knocked out.

FIG. 4A demonstrates the inhibitive activity of BMS-986158 in SW780 cells with either wildtype EP300 or with EP300 knocked out.

FIG. 4B demonstrates the inhibitive activity of BMS-986158 in KP4 cells with either wildtype EP300 or with EP300 knocked out.

FIG. 5A demonstrates the inhibitive activity of GSK525762 in SW780 cells with either wildtype EP300 or with EP300 knocked out.

FIG. 5B demonstrates the inhibitive activity of GSK525762 in KP4 cells with either wildtype EP300 or with EP300 knocked out.

FIG. 6 demonstrates the inhibitive activity of INCB054329 in KP4 cells with either wildtype EP300 or with EP300 knocked out.

FIG. 7A demonstrates the inhibitive activity of OTX-015 in RT11284 cells with either wildtype EP300 or with EP300 knocked out.

FIG. 7B demonstrates the inhibitive activity of OTX-015 in KP4 cells with either wildtype EP300 or with EP300 knocked out.

FIG. 7C demonstrates the inhibitive activity of OTX-015 in SW780 cells with either wildtype EP300 or with EP300 knocked out.

FIG. 7D demonstrates the inhibitive activity of OTX-015 in Calu1 cells with either wildtype EP300 or with EP300 knocked out.

FIG. 8A demonstrates the inhibitive activity of PLX-51107 in RT11284 cells with either wildtype EP300 or with EP300 knocked out.

FIG. 8B demonstrates the inhibitive activity of PLX-51107 in Calu1 cells with either wildtype EP300 or with EP300 knocked out.

FIG. 8C demonstrates the inhibitive activity of PLX-51107 in KP4 cells with either wildtype EP300 or with EP300 knocked out.

FIG. 9 demonstrates the inhibitive activity of TEN-010 in KP4 cells with either wildtype EP300 or with EP300 knocked out.

FIG. 10 illustrates the inhibitive activity of C177 (JQ1) in KP4 cells with either wildtype EP300 or with EP300 knocked out

FIG. 11 illustrates in vivo activity of PLX-51107 and OTX-015 in a bladder cancer PDX model that has an EP300 loss of function mutation.

FIG. 12 illustrates mouse body weight change after administration of PLX-51107 or OTX-015 in the bladder cancer PDX model that has an EP300 loss of function mutation.

FIG. 13 illustrates the inhibitive activity of AZD-5153 in H2009 cells with either wildtype EP300 or with EP300 knocked out.

FIG. 14 illustrates the inhibitive activity of TEN-010 in KP4 cells with either wildtype EP300 or with EP300 knocked out.

FIG. 15 illustrates the inhibitive activity of INCB057643 in KP4 cells with either wildtype EP300 or with EP300 knocked out.

FIG. 16 illustrates the inhibitive activity of INCB057643 in SW780 cells with either wildtype EP300 or with EP300 knocked out.

FIG. 17 illustrates the inhibitive activity of INCB057643 in HCC827 cells with either wildtype EP300 or with EP300 knocked out.

FIG. 18 illustrates the inhibitive activity of CC-90010 in KP4 cells with either wildtype EP300 or with EP300 knocked out.

FIG. 19 illustrates the inhibitive activity of BI-2536 in KP4 cells with either wildtype EP300 or with EP300 knocked out. BI-2536 is a potent Plk1 inhibitor with >40× specificity for Plk1 versus BRD4.

FIG. 20 illustrates the inhibitive activity of PLX-51107 in KP4 cells with either wildtype EP300 or with EP300 knocked out.

FIG. 21 illustrates the inhibitive activity of PLX-51107 in Calu1 cells with either wildtype EP300 or with EP300 knocked out.

FIG. 22 illustrates the inhibitive activity of PLX-51107 in SW780 cells with either wildtype EP300 or with EP300 knocked out.

FIG. 23 illustrates the inhibitive activity of Alobresib in Calu1 cells with either wildtype EP300 or with EP300 knocked out.

FIG. 24 illustrates the inhibitive activity of Alobresib in KP4 cells with either wildtype EP300 or with EP300 knocked out.

FIG. 25 illustrates the inhibitive activity of ODM-207 in KP4 cells with either wildtype EP300 or with EP300 knocked out.

FIG. 26 illustrates the inhibitive activity of GSK778 in KP4 cells with either wildtype EP300 or with EP300 knocked out.

FIG. 27 illustrates the inhibitive activity of GSK778 in Calu1 cells with either wildtype EP300 or with EP300 knocked out.

FIG. 28 illustrates the inhibitive activity of GSK046 in KP4 cells with either wildtype EP300 or with EP300 knocked out.

FIG. 29 illustrates the inhibitive activity of ABBV-744 in Calu1 cells with either wildtype EP300 or with EP300 knocked out.

FIG. 30 illustrates the inhibitive activity of ABBV-744 in KP4 cells with either wildtype EP300 or with EP300 knocked out.

FIG. 31 illustrates the inhibitive activity of Compound 5 in KP4 cells with either wildtype EP300 or with EP300 knocked out.

FIG. 32 illustrates the inhibitive activity of Mivebresib in KP4 cells with either wildtype EP300 or with EP300 knocked out.

FIG. 33 illustrates the inhibitive activity of ZEN-3694 in KP4 cells with either wildtype EP300 or with EP300 knocked out.

FIG. 34 illustrates the inhibitive activity of Trotabresib in KP4 cells with either wildtype EP300 or with EP300 knocked out.

FIG. 35 illustrates the inhibitive activity of Molibresib in KP4 cells with either wildtype EP300 or with EP300 knocked out.

FIG. 36 illustrates the inhibitive activity of Molibresib in SW780 cells with either wildtype EP300 or with EP300 knocked out.

FIG. 37 illustrates the inhibitive activity of Birabresib in Calu1 cells with either wildtype EP300 or with EP300 knocked out.

FIG. 38 illustrates the inhibitive activity of Birabresib in KP4 cells with either wildtype EP300 or with EP300 knocked out.

FIG. 39 illustrates the inhibitive activity of Birabresib in SW780 cells with either wildtype EP300 or with EP300 knocked out.

FIG. 40 illustrates the inhibitive activity of Birabresib in SW1271 cells with either wildtype EP300 or with EP300 knocked out.

FIG. 41 illustrates the inhibitive activity of INCB054329 in KP4 cells with either wildtype EP300 or with EP300 knocked out.

FIG. 42 illustrates the inhibitive activity of BI-894999 in KP4 cells with either wildtype EP300 or with EP300 knocked out.

FIG. 43 illustrates the inhibitive activity of Pelabresib in KP4 cells with either wildtype EP300 or with EP300 knocked out.

FIG. 44 illustrates the inhibitive activity of C177 (JQ1) in KP4 cells with either wildtype EP300 or with EP300 knocked out.

FIG. 45A illustrates a volcano plot depicting the effect size and significance of driver gene changes in a CRISPR knockout/Birabresib pharmacogenetic screen in KP4 cells.

FIG. 45B illustrates a volcano plot depicting the effect size and significance of driver gene changes in a CRISPR knockout/Birabresib pharmacogenetic screen in MDAMB231 triple-negative breast cancer cells.

FIG. 46A illustrates the change in tumor volume over time in PDX model J000108112, a bladder cancer. Mouse models were treated with vehicle, 20 mg/kg Birabresib, or 20 mg/kg PLX-51107.

FIG. 46B illustrates the change in body weight over time in PDX model J000108112, a bladder cancer. Mouse models were treated with vehicle, 20 mg/kg Birabresib, or 20 mg/kg PLX-51107.

FIG. 46C illustrates the change in tumor volume over time in PDX model J000108112, a bladder cancer. Mouse models were treated with vehicle or 20 mg/kg Birabresib.

FIG. 46D illustrates the change in tumor volume over time in PDX model J000108112, a bladder cancer. Mouse models were treated with vehicle or 20 mg/kg PLX-51107.

FIG. 47A illustrates the change in tumor volume over time in PDX model TM00244, a lung cancer. Mouse models were treated with vehicle, 20 mg/kg Birabresib, or 20 mg/kg PLX-51107.

FIG. 47B illustrates the change in body weight over time in PDX model TM00244, a lung cancer. Mouse models were treated with vehicle, 20 mg/kg Birabresib, or 20 mg/kg PLX-51107.

FIG. 47C illustrates the change in tumor volume over time in PDX model TM00244, a lung cancer. Mouse models were treated with vehicle or 20 mg/kg Birabresib.

FIG. 47D illustrates the change in tumor volume over time in PDX model TM00244, a lung cancer. Mouse models were treated with vehicle or 20 mg/kg PLX-51107.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure provides for new methods of treatment or prevention of cancers, resulting in unexpectedly superior cancer cell inhibition or cancer treatment. The methods provided herein may utilize synthetic lethality to overcome challenges associated with BET inhibitors.

Precision medicine may provide some of the newest approaches to cancer treatment. Targeted therapeutics may be designed to exploit disease biology and to define which patients should receive a specific therapy. One concept underlying this approach is that the molecular features of a patient's tumor may influence clinical response to a medication, and thus, may be used to guide therapy (e.g., medication selection). As such, precision medicine may lead to increased efficacy (e.g., increased tumor cell growth inhibition or death) and reduced toxicity.

Tumor genetics may be central to this approach. For example, cancer may be caused by driver mutations, which confer a selective advantage and may allow cancer cells to proliferate. Driver mutations may be used to define patients and may be highly predictive for drug activity; that is, the presence of a specific driver mutation may be an indicator of the effectiveness of a given therapy. Approximately one third of cancers may be caused by driver mutations that induce a gain of function (GOF) in oncogenes and result in a protein product that may be therapeutically targeted. The remaining two thirds of cancers are caused by a loss of function (LOF) mutation in a tumor suppressor gene (TSG). In healthy cells, TSGs may act to control cell growth and division; loss of function mutations in these genes may result in uncontrolled growth and cancer. Such cancers are typically more difficult to target than cancers resulting from GOF mutations, because in cancers resulting from a LOF mutation it is the absence of a functional protein that results in oncogenesis. Thus, a target is more difficult to ascertain. As evidence of this disparity in treatability, drugs exist against approximately ⅔ of oncogenes, but drugs exist for only ~2% of tumor suppressor genes (e.g., the PARPi targeting homologous recombination defects, e.g. BRCA1/2, ATM, ATR, mutations). One potential approach to targeting LOF cancers is to leverage vulnerabilities that are unique to tumor cells by identifying synthetic lethal (SL) interactions. In a synthetic lethality relationship between two genes a loss of function in either, but not both, is tolerated. Since only tumor cells contain the driver mutation, drugs that specifically target the mutation will only impact these tumor cells, and off-target effects, such as toxicities, are expected to be minimal. This approach may be applied to identify both genetic dependencies and small molecule sensitivities of cancer cell lines. As evidence of the importance of this approach, massive and ongoing efforts by large consortia are underway to comprehensively map synthetic lethal networks in hundreds of cancer cell lines with the goal of identifying drug targets and the patients who are expected to respond best to them.

While these efforts have provided valuable insights and identified potential drug candidates, significant challenges remain. For example, many patients do not benefit from targeted therapies because large-scale screens produce new target predictions and feed drug development pipelines at the entry point such that treatment is years away, and the drug(s) identified in screens (e.g., small molecule screens) may not recapitulate genetic perturbation profiles and SL relationships identified in cell lines may not translate to tumors in patients.

A number of systematic approaches, such as the Cancer Dependency Map, have yielded comprehensive maps of synthetic lethality in human cancer cells. One could predict new uses of drugs as a cancer genetically targeted therapy on the basis drug targets that are synthetically lethal with mutations prevalent in cancer patients. This approach, however, is limited, since pharmacologic inhibition rarely recapitulates the genetic deletion of the known drug target. For more information, see: Goncalves E, et al. Drug mechanism-of-action discovery through the integration of pharmacological and CRISPR screens. Mol. Syst. Biol. 2020, 16, e9405 and see, Babak T, et al. Abstract 4035: Driver-gene dependencies reveal clinically actionable drug repositioning opportunities, Cancer Res (2022) 82 (12_Supplement): 4035.

The development of resistance to BET inhibitors is an increasingly growing concern, as resistance to BET inhibitors may emerge early in the treatment of certain cancers. The mechanisms leading to BET inhibitor resistance are multifactorial. Interestingly, none of the reported resistance mechanisms are related to genetic aberrations of the bromodomains (i.e., BRD2/3/4 mutations). In ovarian cancer, prolonged treatment with BET inhibitors has been reported to cause receptor tyrosine kinase reprogramming and subsequently resistance to BET inhibitors. In colorectal cancer, activated interleukin 6/8-Janus kinase 2 signaling was known to promote Brd4 phosphorylation. To this end, phosphorylated Brd4 becomes more stable and binds to BET inhibitors with lower affinity, thus contributing to BET inhibitor resistance. In AML and pancreatic cancer cells, the compensatory upregulation of MYC via the WNT pathway was reported to reduce the responsiveness of the cancer cells to BET inhibitors. Moreover, JQ1-resistant AML cells did not undergo apoptosis but switched to pro-survival autophagy. JQ-1-induced autophagy in the resistant AML cells was associated with the upregulation of Beclin 1, increased LC3-II expression, and accumulation of autophagosomes, which was independent of mTOR signaling. In triple-negative breast cancer, resistance to BET inhibitors was mediated by a bromodomain-independent mechanism. In prostate cancer, the loss-of-function mutation of SPOP (an E3 ubiquitin ligase of Brd4) has been shown to confer resistance to BET inhibitors by impairing ubiquitination-mediated Brd4 degradation. The maintenance of MYC expression was also reported to promote de novo resistance to BET inhibitors in castration-resistant prostate cancer. Following prolonged treatment with CRBN- or VHL-based BET-targeting PROTACs, acquired resistance has been shown to emerge due to genomic alternations that compromise core components of the CRBN or VHL E3 ligase complexes. On the other hand, the hyperphosphorylation of Brd4 due to the downregulation of the phosphatase PP2A and elevated expression ratio of BCL2L1/BCL-XL has also been reported to contribute to BET inhibitor resistance in triple-negative breast cancer. In pancreatic cancer, prolonged treatmentwith JQ-1 has been shown to trigger rebound increase in the BET inhibitor target genes includingFOSLI and HMGA2. Exploring synthetic lethality relationships with BET inhibitors may provide a solution to this concern.

Synthetic lethality is a phenomenon in which the simultaneous loss of function or deletion (or a combination of both) of two genes together leads to cell death, whereas the loss of function of either gene alone does not lead to cell death. This concept may be used in cancer research as a therapeutic strategy for target identification, where a goal is to identify pairs of genes whose combined inhibition leads to cell death in cancer cells while not resulting in the death of non-cancerous cells. An important concept underlying synthetic lethality may be to target a vulnerability in cancer cells that is not present in normal cells. Since cancer cells already have a mutated gene, a promising approach may be to identify and then pharmacologically target a synthetically lethal partner of one of the mutated genes. However, the identification of synthetic lethal gene pairs and the further identification of a pharmaceutical intervention for use in targeting the synthetically lethal partner remains a significant challenge.

In particular embodiments, the present disclosure provides methods and kits for treating a cancer through synthetic lethality. Synthetic lethality may occur with cancers comprising a loss of function mutation in the EP300 gene or a deletion of the EP300 by administration of a BET inhibitor. In some embodiments, the methods provided herein comprises determining the presence of a mutation or deletion of the EP300 gene (e.g., in the subject). In some embodiments, the method comprises treating a cancer comprising a loss of function mutation in a EP300 gene or a deletion of a EP300 gene or a portion thereof. In some embodiments, the loss of function mutation is in a EP300 gene. In some embodiments, the deletion is of the EP300 gene. In some embodiments, the BET inhibitor inhibits BRD2, BRD3, BRD4, or a combination thereof. In some embodiments, the BET inhibitor inhibits BD1 or BD2. In some embodiments, the BET inhibitor inhibits BD2. In some embodiments, the BET inhibitor is a selective BD2 inhibitor. In some embodiments, the selective BD2 inhibitor comprises GSK046, ABBV-744, or Compound 9. In some embodiments, the BET inhibitor is a selective BD1 inhibitor. In some embodiments, the selective BD1 inhibitor comprises GSK778.

### I. Methods of Treating Cancer

Provided herein, in some embodiments, are methods of treating a cancer in a subject in need thereof, said methods comprising administering a pharmaceutical composition to said subject, wherein said pharmaceutical composition comprises a bromodomain and extra-terminal domain (BET) inhibitor and said cancer has previously been determined to comprise a loss of function or a deletion of an EP300 gene. In some embodiments, said loss of function is caused by genetic mutation. In some embodiments, said patient is not concurrently receiving

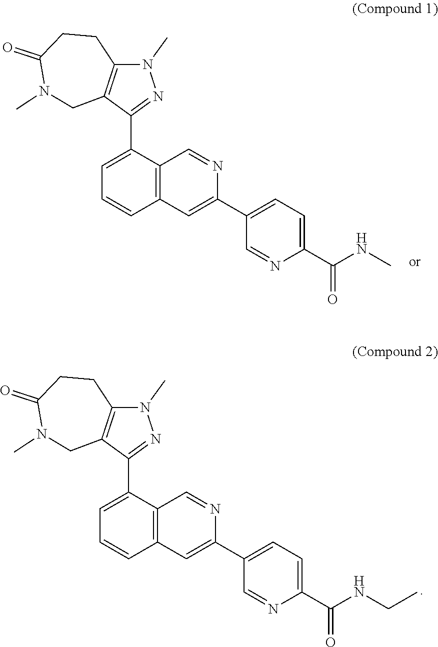 [SMILES: CNC(=O)c1ccc(-c2cc3cccc(-c4nn(C)c5c4CN(C)C(=O)CC5)c3cn2)cn1 ; CCNC(=O)c1ccc(-c2cc3cccc(-c4nn(C)c5c4CN(C)C(=O)CC5)c3cn2)cn1]

In some embodiments, the method includes a proviso that the BET inhibitor does not inhibit a CREB binding protein (CREBBP). In some embodiments, the method includes a proviso that the BET inhibitor does not inhibit a Plk1. In some embodiments, the BET inhibitor is at least 10× more selective for BET than Plk1. In some embodiments, the method includes a proviso that the BET inhibitor is not JQ1. In some embodiments, the method includes a proviso that the cancer is not triple negative breast cancer. In some embodiments, the method includes a proviso that the BET inhibitor is not JQ1 and that the cancer is not triple negative breast cancer.

Further provided herein, in some embodiments, are methods of treating a cancer in a subject in need thereof, said methods comprising administering a pharmaceutical composition to said subject, wherein said pharmaceutical composition comprises a bromodomain and extra-terminal domain (BET) inhibitor; said cancer has previously been determined to comprise a decreased amount or activity of p300 compared to wild type p300; and said patient is not concurrently receiving or has not previously received Compound 1 or Compound 2. In some embodiments, said cancer has previously been determined to comprise a decreased amount of p300 compared to wild type p300. In some embodiments, said cancer has previously been determined to comprise a decreased activity of p300 compared to wild type p300. In some embodiments, the method includes a proviso that the BET inhibitor does not inhibit a CREB binding protein (CREBBP). In some embodiments, the method includes a proviso that the BET inhibitor does not inhibit a Plk1. In some embodiments, the method includes a proviso that the BET inhibitor is not JQ1. In some embodiments, the method includes a proviso that the cancer is not triple negative breast cancer. In some embodiments, the method includes a proviso that the BET inhibitor is not JQ1 and that the cancer is not triple negative breast cancer.

In some embodiments, said subject is not concurrently receiving Compound 1 or Compound 2 and said BET inhibitor. In some embodiments, said subject has not previously received Compound 1 or Compound 2. In some embodiments, said BET inhibitor comprises ABBV-075, ABBV-744, Apabetalone, APL-581, ARV-825, AZD-5153, BI-6727, BI-894999, BMS-986158, BOS-475, BPI-23314, CD-161, CG-223, CK-103, CN-470, FT-1101, GNE-0011, GS-5829, GS-626510, GSK525762, I-BET151, INCB054329, INCB57643, JQ1LY-294002, NE02734, ODM-207, OMT-001, OMT-002, OTX-015, Pelabresib, PLX-2853 (OPN-2853), PLX-51107 (OPN-51107), TEN-010, RVX-297, SRX-2523, SRX-3225, SRX-3254, SW-064652, SYHA-1801, TTI-281, Zen-3694, CPI-0610, INCB0543294, JAB-8263, INCB057643, CC-90010, Alobresib, GSK778, GSK046, Mivebresib, Trotabresib, Molibresib, Pelabresib, or a combination thereof. In some embodiments, said BET inhibitor comprises AZD-5153, BI-894999, BMS-986158, GSK525762, INCB054329, INCB57643, OTX-015, PLX-51107, or a combination thereof. In some embodiments, said BET inhibitor comprises Compound 5. In some embodiments, said BET inhibitor comprises Compound 9. In some embodiments, said BET inhibitor comprises AZD-5153, BI-894999, BMS-986158, GSK525762, INCB054329, OTX-015, PLX-51107, INCB057643, TEN-010, CC-90010, Alobresib, ODM-207, GSK778, GSK046, ABBV-744, Mivebresib, Trotabresib, Molibresib, Birabresib, Pelabresib, or a combination thereof. In some embodiments, said BET inhibitor is a small molecule compound.

In some embodiments, said BET inhibitor comprises a compound of Formula (I):

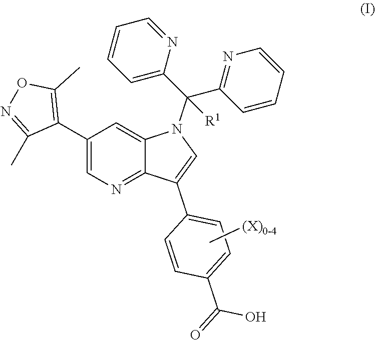

or a pharmaceutically acceptable salt, a tautomer, a stereoisomer or a deuterated analog thereof, wherein: R¹ is cyano, halo, or (C₁-C₃)alkyl optionally substituted with 1 to 3 substituents independently selected from the group consisting of halo, methyl, ethyl, methoxy and ethoxy; and X, when present, is halo. In some embodiments, R¹ is (C₁-C₂)alkyl, cyano or fluoro. In some embodiments, R¹ is methyl. In some embodiments, R¹ is fluoro. In some embodiments, R¹ is cyano.

In some embodiments, said BET inhibitor comprises a compound of Formula (II):

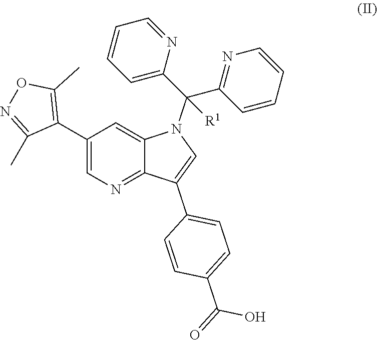

or a pharmaceutically acceptable salt, a tautomer, a stereoisomer, or a deuterated analog thereof, wherein: R¹ is (C₁-C₃)alkyl optionally substituted with 1 to 3 substituents independently selected from the group consisting of halo, methyl, ethyl, methoxy and ethoxy. In some embodiments, R¹ is methyl.

In some embodiments, said BET inhibitor comprises a compound of Formula (III):

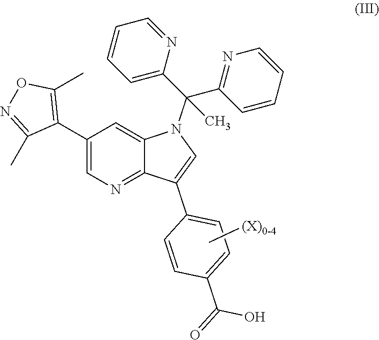

or a pharmaceutically acceptable salt thereof, wherein: X, when present, is halo.

In some embodiments, said BET inhibitor comprises a compound of formula IV:

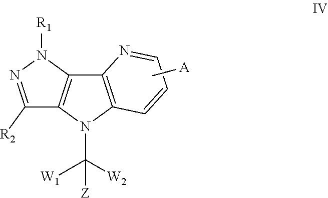

a pharmaceutically acceptable salt thereof or a stereoisomer thereof, wherein,
R₁ is selected from hydrogen; deuterium; —C₁₋₆alkyl; or —C₃₋₈carbocyclic; and each of which at each occurrence is independently optionally substituted with 1, 2, 3, 4, 5 or 6 substituents, and the said each of substituents at each occurrence is independently selected from deuterium, halogen, OH, —CN, —C₁₋₈ alkyl, —C₁₋₈alkoxy, —NH₂, —NH(C₁₋₆alkyl), —N(C₁₋₆ alkyl)₂, or —C₃₋₈ carbocyclic;
R₂ is selected from hydrogen; deuterium; halogen; —OR₂₁; —NR₂₁R₂₂; —CN; —SR₂₁; —SOR₂₁; —SO₂R₂₁; —SO₂NR₂₁R₂₂; —C₁₋₈alkyl;

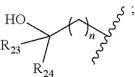

carboxyl; —COOR₂₁; —CONR₂₁R₂₂; —NR₂₁COR₂₂; —NR₂₁SO₂R₂₂; or —C₃₋₈carbocyclic; and each of which at each occurrence is independently optionally substituted with 1, 2, 3, 4, 5 or 6 substituents, and the said each of substituents at each occurrence is independently selected from deuterium, halogen, —OH, —CN, —NH₂, —C₁₋₈alkyl, —C₁₋₈alkoxy, —C₃₋₈carbocyclic, or 6-membered heterocyclic containing 1, 2 or 3 heteroatoms selected from N, O;
each of R₂₁ and R₂₂ at each occurrence is independently selected from hydrogen; deuterium; —OH; NH₂; —CN; —C₁₋₈alkyl; —C₁₋₈alkoxy; —C₁₋₈alkylene-C₃₋₈ carbocyclic; or —C₃₋₈carbocyclic;
each of R₂₃ and R₂₄ at each occurrence is independently selected from hydrogen, deuterium, or —C₁₋₈alkyl;
   A is selected from

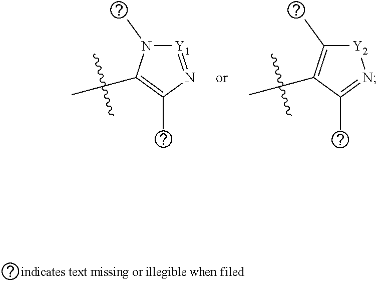

Y₁ is selected from N or CR_{Y1};
Y₂ is selected from O, S, CR_{Y1}R_{Y2} or NR_{Y2};
   each of R_{Y1} and R_{Y2} at each occurrence is independently selected from hydrogen, deuterium, halogen, —OH, NH₂, —CN, —C₁₋₆alkyl or —C₁₋₆alkoxy;
each of R₃ and R₄ at each occurrence is independently selected from hydrogen, deuterium, or —C₁₋₆alkyl; and each of which at each occurrence is independently optionally substituted with 1, 2, 3, 4, 5 or 6 substituents, and the said each of substituents at each occurrence is independently selected from deuterium, halogen, —OH, —NH₂, —CN, —C₁₋₆alkyl or —C₁₋₆alkoxy;
n is selected from 0, 1, 2, 3, 4, 5 or 6;
W₁ is selected from hydrogen; deuterium; —F; —CI; —NH₂; —CN; —OH; carboxyl; —C₁₋₆ alkyl; —C₁₋₆alkoxy; —C₁₋₃alkylene-C₁₋₃alkoxy; phenyl; 5-membered heteroaryl containing 1, 2 or 3 heteroatoms selected from N, O; 6-membered heteroaryl containing 1, 2 or 3 heteroatoms selected from N, O; 3-membered heterocyclic containing 1, 2 or 3 heteroatoms selected from N, O; 4-membered heterocyclic containing 1, 2 or 3 heteroatoms selected from N, O; 5-membered heterocyclic containing 1, 2 or 3 heteroatoms selected from N, O; 6-membered heterocyclic containing 1, 2 or 3 heteroatoms selected from N, O; 3-membered carbocyclic; 4-membered carbocyclic; 5-membered carbocyclic; or 6-membered carbocyclic; and each of which at each occurrence is independently optionally substituted with 1, 2, 3, 4, 5 or 6 substituents, and the said each of substituents at each occurrence is selected from deuterium, halogen, —NH₂, —CN, —OH, —NO₂, carboxyl, —C₁₋₃ alkyl, or —C₁₋₃alkoxy;
W₂ is selected from hydrogen; deuterium; —F; —CI; —NH₂; —CN; —OH; carboxyl; —C₁₋₃ alkyl; —C₁₋₃alkoxy; phenyl; naphthyl; 5-membered heteroaryl containing 1, 2 or 3 heteroatoms selected from N, O or S; 6-membered heteroaryl containing 1, 2 or 3 heteroatoms selected from N, O, or S; 7-membered heteroaryl containing 1, 2 or 3 heteroatoms selected from N, O or S; 8-membered heteroaryl containing 1, 2 or 3 heteroatoms selected from N, O or S; 9-membered heteroaryl containing 1, 2 or 3 heteroatoms selected from N, O or S; 10-membered heteroaryl containing 1, 2 or 3 heteroatoms selected from N, O, or S; 3-membered heterocyclic containing 1, 2 or 3 heteroatoms selected from N, O or S; 4-membered heterocyclic containing 1, 2 or 3 heteroatoms selected from N, O or S; 5-membered heterocyclic containing 1, 2 or 3 heteroatoms selected from N, O or S; 6-membered heterocyclic containing 1, 2 or 3 heteroatoms selected from N, O or S; 3-membered carbocyclic; 4-membered carbocyclic; 5-membered carbocyclic; or 6-membered carbocyclic; and each of which at each occurrence is independently optionally substituted with 1, 2, 3, 4 or 5 substituents, and the said each of substituents at each occurrence is selected from deuterium, halogen, —NH₂, —CN, —OH, —NO₂, carboxyl, methyl, ethyl, propyl, isopropyl, methoxy, ethoxy, propoxy or isopropoxy;
Z is selected from hydrogen, deuterium, halogen, —NH₂, —CN, —OH, carboxyl, —C₁₋₆alkyl or —C₁₋₆alkoxy.

In some embodiments, R1 is selected from hydrogen; deuterium; —C1-6alkyl; or —C3-₈carbocyclic; and each of which at each occurrence is independently optionally substituted with 1, 2, 3, 4, 5 or 6 substituents, and the said each of substituents at each occurrence is independently selected from deuterium, halogen, OH, —CN, —C₁₋₈alkyl, or —C₁₋₈alkoxy;
R₂ is selected from hydrogen; deuterium; halogen; —C₁₋₈alkyl;

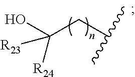

carboxyl; —COOR₂₁; or —CONR₂₁R₂₂; and each of which at each occurrence is independently optionally substituted with 1, 2, 3, 4, 5 or 6 substituents, and the said each of substituents at each occurrence is independently selected from deuterium, halogen, —OH, —CN, —NH₂, —C₁₋₈alkyl, —C₁₋₈alkoxy, C₃₋₈carbocyclic, or 6-membered heterocyclic containing 1, 2 or 3 heteroatoms selected from N, O;
each of R₂₁ and R₂₂ at each occurrence is independently selected from hydrogen; deuterium; —OH; NH₂; —CN; —C₁₋₈alkyl; or —C₃₋₈carbocyclic;
each of R₂₃ and R₂₄ at each occurrence is independently selected from hydrogen, deuterium, or —C₁₋₈alkyl;
   A is selected from

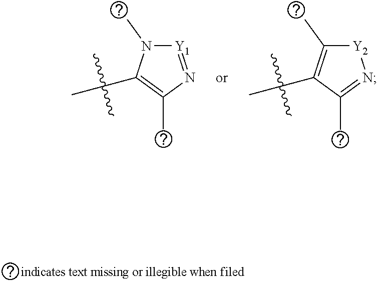

Y₁ is selected from N or CR_{Y1};
Y₂ is selected from O, S, CR_{Y1}R_{Y2} or NR_{Y2};
   each of R_{Y1} and R_{Y2} at each occurrence is independently selected from hydrogen, deuterium, halogen, —OH, NH₂, —CN, —C₁₋₆alkyl or —C₁₋₆alkoxy;
each of R₃ and R₄ at each occurrence is independently selected from hydrogen, deuterium, or —C₁₋₆alkyl; and each of which at each occurrence is independently optionally substituted with 1, 2, 3, 4, 5 or 6 substituents, and the said each of substituents at each occurrence is independently selected from deuterium, halogen, —OH, —NH₂, —CN, —C₁₋₆alkyl or —C₁₋₆alkoxy;
n is selected from 0, 1 or 2;
W₁ is selected from hydrogen; deuterium; —F; —CI; —NH₂; —CN; —OH; carboxyl; —C₁₋₆alkyl; —C₁₋₆alkoxy; 6-membered heterocyclic containing 1, 2 or 3 heteroatoms selected from N, O; and each of which at each occurrence is independently optionally substituted with 1, 2, 3, 4, 5 or 6 substituents, and the said each of substituents at each occurrence is selected from deuterium, halogen, —NH₂, —CN, —OH, —NO₂, carboxyl, —C₁₋₃alkyl, or —C₁₋₃alkoxy;
W₂ is selected from hydrogen; deuterium; —F; —CI; —NH₂; —CN; —OH; carboxyl; —C₁₋₃alkyl; —C₁₋₃alkoxy; phenyl; naphthyl; 5-membered heteroaryl containing 1, 2 or 3 heteroatoms selected from N, O or S; 6-membered heteroaryl containing 1, 2 or 3 heteroatoms selected from N, O, or S; and each of which at each occurrence is independently optionally substituted with 1, 2, 3, 4 or 5 substituents, and the said each of substituents at each occurrence is selected from deuterium, halogen, —NH₂, —CN, —OH, —NO₂, carboxyl, methyl, ethyl, propyl, isopropyl, methoxy, ethoxy, propoxy or isopropoxy;
Z is selected from hydrogen, deuterium, halogen, —NH₂, —CN, —OH, or —C₁₋₆alkoxy.

In some embodiments, R1 is selected from hydrogen; deuterium; or —C₁₋₆alkyl. In some embodiments, R1 is selected from hydrogen; In some embodiments, R1 is selected from deuterium. In some embodiments, R1 is selected from —C1-6alkyl.

In some embodiments, R₂ is selected from hydrogen; deuterium; halogen; —C₁₋₈alkyl;

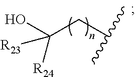

or —CONR₂₁R₂₂; and each of which at each occurrence is independently optionally substituted with 1, 2, 3, 4, 5 or 6 substituents, and the said each of substituents at each occurrence is independently selected from deuterium, halogen, —OH, —CN, —NH₂, —C₁₋₈alkyl, —C₁₋₈alkoxy, C₃₋₈carbocyclic, or 6-membered heterocyclic containing 1, 2 or 3 heteroatoms selected from N, O. In some embodiments, R₂ is selected from hydrogen; deuterium; halogen; —C₁₋₈alkyl; and each of which at each occurrence is independently optionally substituted with 1, 2, 3, 4, 5 or 6 substituents, and the said each of substituents at each occurrence is independently selected from deuterium, halogen, —OH, —CN, —NH₂, —C₁₋₈alkyl, —C₁₋₈alkoxy, C₃₋₈carbocyclic, or 6-membered heterocyclic containing 1, 2 or 3 heteroatoms selected from N, O. In some embodiments, R₂ is selected from hydrogen; —C₁₋₈alkyl; and each of which at each occurrence is independently optionally substituted with 1, 2, 3, 4, 5 or 6 substituents, and the said each of substituents at each occurrence is independently selected from deuterium, halogen, —OH, —CN, —NH₂, —C₁₋₈alkyl, —C₁₋₈alkoxy, C₃₋₈carbocyclic, or 6-membered heterocyclic containing 1, 2 or 3 heteroatoms selected from N, O; In some embodiments, R₂ is selected from —C₁₋₈alkyl; which at each occurrence is independently optionally substituted with 1, 2, 3, 4, 5 or 6 substituents, and the said each of substituents at each occurrence is independently selected from deuterium, halogen, —OH, —CN, —NH₂, —C₁₋₈alkyl, —C₁₋₈alkoxy, C₃₋₈carbocyclic, or 6-membered heterocyclic containing 1, 2 or 3 heteroatoms selected from N, O. In some embodiments, each of R₂₁ and R₂₂ at each occurrence is independently selected from hydrogen; deuterium; —OH; NH₂; or —C₃₋₈carbocyclic. In some embodiments, each of R₂₁ and R₂₂ at each occurrence is independently selected from hydrogen; or deuterium. In some embodiments, R₂₁ and R₂₂ are selected from hydrogen.

In some embodiments, each of R₂₃ and R₂₄ at each occurrence is independently selected from hydrogen, or —C₁₋₈alkyl. In some embodiments, each of R₂₃ and R₂₄ at each occurrence is independently selected from —C₁₋₂alkyl.

In some embodiments, A is selected from

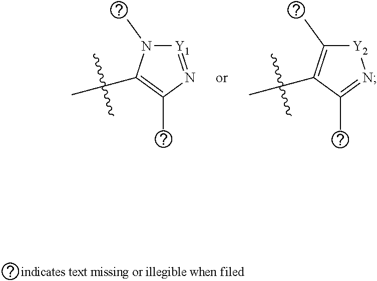

In some embodiments, A is selected from

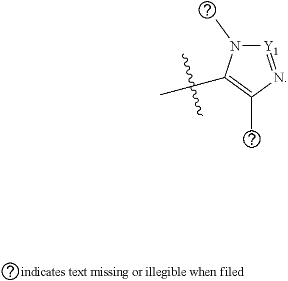

In some embodiments, A is selected from

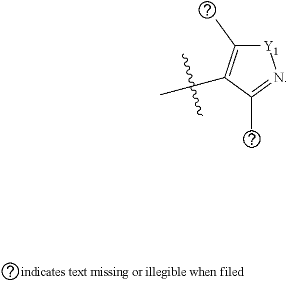

In some embodiments, Y₁ is selected from N or CR_{Y1}. In some embodiments, Y₁ is selected from N. In some embodiments, Y₁ is selected from CR_{Y1}. In some embodiments, Y₂ is selected from O, S, CR_{Y1}R_{Y2} or NR_{Y2}. In some embodiments, Y₂ is selected from 0. In some embodiments, Y₂ is selected from S. In some embodiments, each of R_{Y1} and R_{Y2} at each occurrence is independently selected from hydrogen, deuterium, or —C₁₋₆alkyl.

In some embodiments, each of R₃ and R₄ at each occurrence is independently selected from hydrogen, deuterium, or —C₁₋₆alkyl; and each of which at each occurrence is independently optionally substituted with 1, 2, 3, 4, 5 or 6 substituents, and the said each of substituents at each occurrence is independently selected from deuterium, halogen, or —C₁₋₆alkoxy. In some embodiments, each of R₃ and R₄ at each occurrence is independently selected from —C₁₋₆alkyl; and each of which at each occurrence is independently optionally substituted with 1, 2, 3, 4, 5 or 6 substituents, and the said each of substituents at each occurrence is independently selected from deuterium.

In some embodiments, n is selected from 1 or 2. In some embodiments, n is selected from 1. In some embodiments, n is selected from 2.

In some embodiments, W₁ is selected from hydrogen; deuterium; —F; —CI; —NH₂; —CN; —OH; carboxyl; —C₁₋₆alkyl; —C₁₋₆alkoxy; 6-membered heterocyclic containing 1, 2 or 3 heteroatoms selected from N, O; and each of which at each occurrence is independently optionally substituted with 1, 2, 3, 4, 5 or 6 substituents, and the said each of substituents at each occurrence is selected from deuterium, halogen, —NH₂, —CN, or —OH. In some embodiments, W₁ is selected from —C₁₋₆alkyl; 6-membered heterocyclic containing 1 or 2 heteroatoms selected from 0; and each of which at each occurrence is independently optionally substituted with 1, 2, 3, 4, 5 or 6 substituents, and the said each of substituents at each occurrence is selected from deuterium, halogen, or —OH. In some embodiments, W₁ is selected from —C₁₋₆alkyl; 6-membered heterocyclic containing 1 heteroatom selected from 0; and each of which at each occurrence is independently optionally substituted with 1, 2, 3, 4, 5 or 6 substituents, and the said each of substituents at each occurrence is selected from halogen.

In some embodiments, W₂ is selected from hydrogen; deuterium; —F; —CI; —NH₂; —CN; —OH; carboxyl; —C₁₋₃alkyl; —C₁₋₃alkoxy; phenyl; 6-membered heteroaryl containing 1, 2 or 3 heteroatoms selected from N, O, or S; and each of which at each occurrence is independently optionally substituted with 1, 2, 3, 4 or 5 substituents, and the said each of substituents at each occurrence is selected from deuterium, halogen, —NH₂, —CN, —OH, —NO₂, carboxyl, methyl, ethyl, propyl, isopropyl, methoxy, ethoxy, propoxy or isopropoxy. In some embodiments, W₂ is selected from phenyl; 6-membered heteroaryl containing 1, 2 or 3 heteroatoms selected from N; and each of which at each occurrence is independently optionally substituted with 1, 2, 3, 4 or 5 substituents, and the said each of substituents at each occurrence is selected from deuterium, halogen, methyl, or methoxy. In some embodiments, W₂ is selected from phenyl; which at each occurrence is independently optionally substituted with 1, 2, 3, 4 or 5 substituents, and the said each of substituents at each occurrence is selected from deuterium, halogen, methyl, or methoxy. In some embodiments, W₂ is selected from 6-membered heteroaryl containing 1, 2 or 3 heteroatoms selected from N; which at each occurrence is independently optionally substituted with 1, 2, 3, 4 or 5 substituents, and the said each of substituents at each occurrence is selected from deuterium, halogen, methyl, or methoxy.

In some embodiments, Z is selected from hydrogen, deuterium, or halogen. In some embodiments, Z is selected from hydrogen or halogen. In some embodiments, Z is selected from hydrogen.

In some embodiments, said BET inhibitor comprises Compound 5:

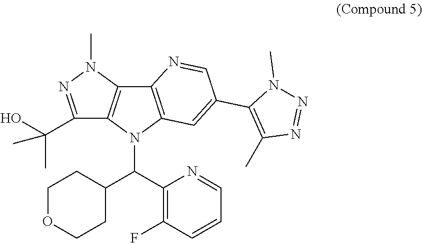 [SMILES: Cc1nnn(C)c1-c1cnc2c3c(c(C(C)(C)O)nn3C)n(C(c3ncccc3F)C3CCOCC3)c2c1]

or a pharmaceutically acceptable salt, a tautomer, a solvate, or a deuterated analog thereof.

In some embodiments, said BET inhibitor comprises Compound 6:

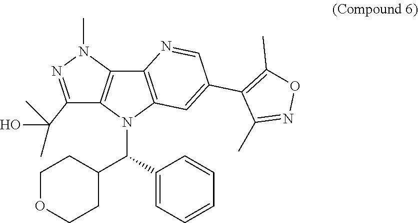 [SMILES: Cc1noc(C)c1-c1cnc2c3c(c(C(C)(C)O)nn3C)n([C@H](c3ccccc3)C3CCOCC3)c2c1]

or a pharmaceutically acceptable salt, a tautomer, a solvate, or a deuterated analog thereof.

In some embodiments, said BET inhibitor comprises Compound 7:

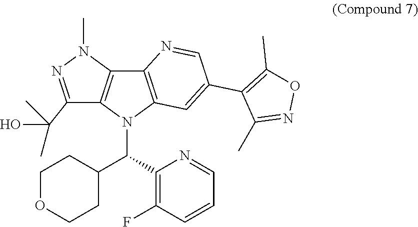 [SMILES: Cc1noc(C)c1-c1cnc2c3c(c(C(C)(C)O)nn3C)n([C@H](c3ncccc3F)C3CCOCC3)c2c1]

or a pharmaceutically acceptable salt, a tautomer, a solvate, or a deuterated analog thereof.

In some embodiments, said BET inhibitor comprises Compound 8:

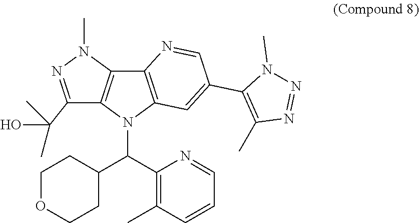 [SMILES: Cc1cccnc1C(C1CCOCC1)n1c2cc(-c3c(C)nnn3C)cnc2c2c1c(C(C)(C)O)nn2C]

or a pharmaceutically acceptable salt, a tautomer, a solvate, or a deuterated analog thereof.

In some embodiments, said BET inhibitor is selected from:
(S)-2-(6-(3,5-dimethylisoxazol-4-yl)-1-methyl-4-(phenyl(tetrahydro-2H-pyran-4-yl)methyl)-1,4-dihydropyrazolo[3′,4′:4,5]pyrrolo[3,2-b]pyridin-3-yl)propan-2-ol;
(S)-2-(6-(3,5-dimethylisoxazol-4-yl)-4-((3-fluoropyridin-2-yl)(tetrahydro-2H-pyran-4-yl)methyl)-1-methyl-1,4-dihydropyrazolo[3′,4′:4,5]pyrrolo[3,2-b]pyridin-3-yl)propan-2-ol;
2-(6-(1,4-Dimethyl-1H-1,2,3-triazol-5-yl)-1-methyl-4-(4,4,4-trifluoro-1-(3-fluoropyridin-2-yl)butyl)-1,4-dihydropyrazolo[3′,4′:4,5]pyrrolo[3,2-b]pyridine-3-yl)propan-2-ol;
2-(6-(1,4-Dimethyl-1H-1,2,3-triazol-5-yl)-1-methyl-4-((3-methylpyridin-2-yl)(tetrahydro-2H-pyran-4-yl)methyl)-1,4-dihydropyrazolo[3′,4′:4,5]pyrrolo[3,2-b]pyridine-3-yl)propan-2-ol;
(S)-2-(6-(1,4-dimethyl-1H-1,2,3-triazol-5-yl)-1-methyl-4-(phenyl(tetrahydro-2H-pyran-4-yl)methyl)-1,4-dihydropyrazolo[3′,4′:4,5]pyrrolo[3,2-b]pyridine-3-yl)propan-2-ol;
(S)-2-(6-(1,4-dimethyl-1H-1,2,3-triazol-5-yl)-4-((3-fluoropyridin-2-yl)(tetrahydro-2H-pyran-4-yl)methyl)-1-methyl-1,4-dihydropyrazolo[3′,4′:4,5]pyrrolo[3,2-b]pyridin-3-yl)propan-2-ol;
2-(6-(3,5-dimethylisoxazol-4-yl)-1-methyl-4-((tetrahydro-2H-pyran-4-yl)(o-tolyl)methyl)-1,4-dihydropyrazolo[3′,4′:4,5]pyrrolo[3,2-b]pyridin-3-yl)propan-2-ol;
(S)-2-(6-(1,4-Dimethyl-1H-1,2,3-triazol-5-yl)-4-((3-fluoropyridin-2-yl)(tetrahydro-2H-pyran-4-yl)methyl)-1,4-dihydropyrazolo[3′,4′:4,5]pyrrolo[3,2-b]pyridin-3-yl)propan-2-ol;
6-(1,4-Dimethyl-1H-1,2,3-triazol-5-yl)-1-methyl-4-(phenyl(tetrahydro-2H-pyran-4-yl)methyl)-1,4-dihydropyrazolo[3′,4′:4,5]pyrrolo[3,2-b]pyridine-3-carboxamide;
2-(6-(1,4-Dimethyl-1H-1,2,3-triazol-5-yl)-1-methyl-4-(phenyl(tetrahydro-2H-pyran-4-yl)methyl)-1,4-dihydropyrazolo[3′,4′:4,5]pyrrolo[3,2-b]pyridine-3-yl)propan-2-amine;
2-(4-((3-Fluoropyridin-2-yl)(tetrahydro-2H-pyran-4-yl)methyl)-1-methyl-6-(1-methyl-4-(methyl-d3)-1H-1,2,3-triazol-5-yl)-1,4-dihydropyrazolo[3′,4′:4,5]pyrrolo[3,2-b]pyridine-3-yl)propan-2-ol;
2-(6-(1,4-Dimethyl-1H-1,2,3-triazol-5-yl)-4-((3-fluoropyridin-4-yl)(tetrahydro-2H-pyran-4-yl)methyl)-1-methyl-1,4-dihydropyrazolo[3′,4′:4,5]pyrrolo[3,2-b]pyridine-3-yl)propan-2-ol;
2-(6-(1,4-Dimethyl-1H-1,2,3-triazol-5-yl)-4-((3-methoxypyridin-2-yl)(tetrahydro-2H-pyran-4-yl)methyl)-1-methyl-1,4-dihydropyrazolo[3′,4′:4,5]pyrrolo[3,2-b]pyridin-3-yl)propan-2-ol; and
4-((6-(1,4-dimethyl-1H-1,2,3-triazol-5-yl)-1-methyl-4-(phenyl(tetrahydro-2H-pyran-4-yl)methyl)-1,4-dihydropyrazolo[3′,4′:4,5]pyrrolo[3,2-b]pyridin-3-yl)methyl)morpholine.

In some embodiments, said BET inhibitor comprises (S)-2-(6-(1,4-dimethyl-1H-1,2,3-triazol-5-yl)-4-((3-fluoropyridin-2-yl)(tetrahydro-2H-pyran-4-yl)methyl)-1-methyl-1,4-dihydropyrazolo[3′,4′:4,5]pyrrolo[3,2-b]pyridin-3-yl)propan-2-ol.

In some embodiments, said BET inhibitor comprises a compound having formula (Va):

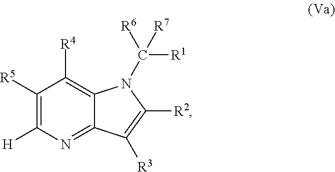

or a pharmaceutically acceptable salt, a solvate, a tautomer, a stereoisomer or a deuterated analog thereof, wherein: R² is H; R⁴ is H; R⁶ is H; R⁷ is H, —OH, C₁₋₆ alkyl, aryl, heteroaryl, cycloalkyl, or heterocycloalkyl;
R¹ is heteroaryl optionally substituted with from 1-3 R^{j} groups;
each R^{j} independently selected from halogen, —CN, —OH, —NH₂, —NO₂, —C(O)OH, —C(S)OH, —C(O)NH₂, —C(S)NH₂, —S(O)₂NH₂, —NHC(O)NH₂, —NHC(S)NH₂, —NHS(O)₂NH₂, —C(NH)NH₂, —CH═C(R^{k})(R^{k}), —OR^{k}, —SR^{k}, —OC(O)R^{k}, —OC(S)R^{k}, —P(═O)HR^{k}, —P(═O)R^{k}R^{k}, —PH(═O)OR^{k}, —P(═O)(OR^{k})₂, —OP(═O)(OR^{k})₂, —C(O)H, —O(CO)OR^{k}, —C(O)R^{k}, —C(S)R^{k}, —C(O)OR^{k}, —C(S)OR^{k}, —S(O)R^{k}, —S(O)₂R^{k}, —C(O)NHR^{k}, —C(S)NHR^{k}, —C(O)NR^{k}R^{k}, —C(S)NR^{k}R^{k}, —S(O)₂NHR^{k}, —S(O)₂NR^{k}R^{k}, —C(NH)NHR^{k}, —C(NH)NR^{k}R^{k}, —NHC(O)R^{k}, —NHC(S)R^{k}, —NR^{k}C(O)R^{k}, —NR^{k}C(S)R^{k}, —NHS(O)₂R^{k}, —NR^{k}S(O)₂R^{k}, —NHC(O)NHR^{k}, —NHC(S)NHR^{k}, —NR^{k}C(O)NH₂, —NR^{k}C(S)NH₂, —NR^{k}C(O)NHR^{k}, —NR^{k}C(S)NHR^{k}, —NHC(O)NR^{k}R^{k}, —NHC(S)NR^{k}R^{k}, —NR^{k}C(O)NR^{k}R^{k}, —NR^{k}C(S)NR^{k}R^{k}, —NHS(O)₂NHR^{k}, —NR^{k}S(O)₂NH₂, —NR^{k}S(O)₂NHR^{k}, —NHS(O)₂NR^{k}R^{k}, —NR^{k}S(O)₂NR^{k}R^{k}, —NHR^{k} or —NR^{k}R^{k};
each R^{k} is independently H, —C₁₋₆alkyl, aryl, arylalkyl, heteroaryl, heteroarylalkyl, heterocycloalkyl, heterocycloalkylalkyl, cycloalkyl or cycloalkylalkyl; or
two R^{k} groups when attached to the same carbon or nitrogen atom are taken together to form a 3- to 6-membered carbocyclic ring or 3- to 8-membered heterocyclic ring having from 1-2 heteroatoms as ring members selected from O, N or S, wherein the nitrogen or sulfur ring atoms are optionally oxidized;
R³ is H, halogen, —CN, optionally substituted C₁₋₆alkyl, optionally substituted deuterated C₁₋₆alkyl, optionally substituted aryl, optionally substituted aryl-C₁₋₄alkyl, optionally substituted heteroaryl, optionally substituted heteroaryl-C₁₋₄alkyl, optionally substituted C₃₋₈ cycloalkyl, optionally substituted C₃₋₈ cycloalkyl-C₁₋₄alkyl, optionally substituted heterocycloalkyl, or optionally substituted heterocycloalkyl-C₁₋₄alkyl; and
R⁵ is

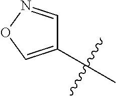 [SMILES: *c1cnoc1]

optionally substituted with from 1 to 2 R¹¹ groups independently selected from D, halogen, C₁₋₆alkyl, C₁₋₄haloalkyl, C₁₋₄haloalkoxy or —CN;
wherein the wavy line indicates the point of attachment to the rest of molecule.

In some embodiments, said BET inhibitor comprises a compound having formula (VI):

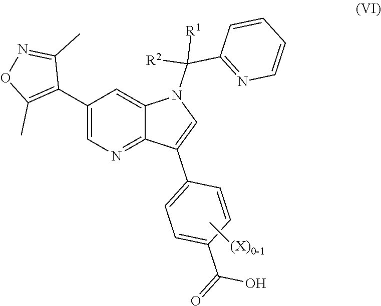

or a pharmaceutically acceptable salt, a tautomer, a stereoisomer or a deuterated analog thereof, wherein: R¹ is cyano, halo, or (C₁-C₃)alkyl optionally substituted with 1 to 3 substituents independently selected from the group consisting of halo, methyl, ethyl, methoxy and ethoxy; X, when present, is halo; R² is H, (C₁-C₃)alkyl, aryl, heteroaryl, 5-membered heteroaryl containing 1, 2 or 3 heteroatoms selected from N, O; 6-membered heteroaryl containing 1, 2 or 3 heteroatoms selected from N, O. In some embodiments, R¹ is (C₁-C₂)alkyl, cyano or fluoro. In some embodiments, R¹ is methyl. In some embodiments, R¹ is fluoro. In some embodiments, R¹ is cyano. In some embodiments, R² is H. In some embodiments, R² is 6-membered heteroaryl containing 1, 2 or 3 heteroatoms selected from N, O. In some embodiments, said BET inhibitor comprises Compound 3:

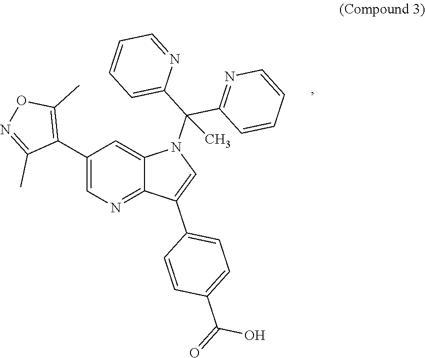 [SMILES: Cc1noc(C)c1-c1cnc2c(-c3ccc(C(=O)O)cc3)cn(C(C)(c3ccccn3)c3ccccn3)c2c1]

or a pharmaceutically acceptable salt, a tautomer, a solvate, or a deuterated analog thereof.

In some embodiments, said BET inhibitor comprises Compound 4:

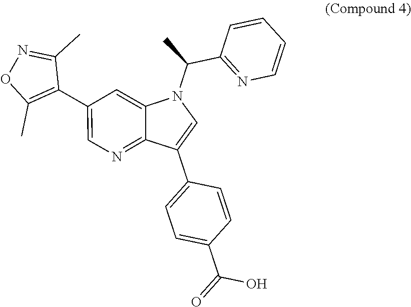 [SMILES: Cc1noc(C)c1-c1cnc2c(-c3ccc(C(=O)O)cc3)cn([C@@H](C)c3ccccn3)c2c1]

or a pharmaceutically acceptable salt, a tautomer, a solvate, or a deuterated analog thereof.

In some embodiments, said BET inhibitor comprises a compound having formula (VII):

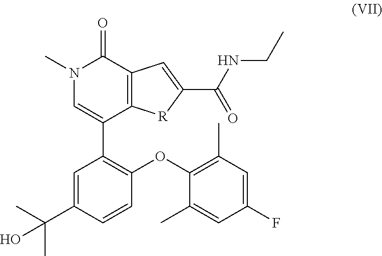

or a pharmaceutically acceptable salt, a solvate, a tautomer, a stereoisomer or a deuterated analog thereof, wherein R is N, O, or S.

In some embodiments, said BET inhibitor comprises Compound 9:

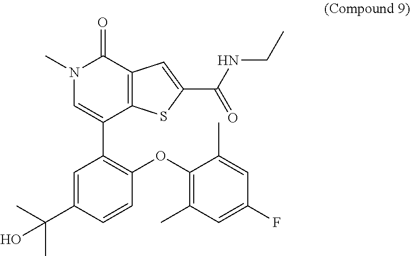 [SMILES: CCNC(=O)c1cc2c(=O)n(C)cc(-c3cc(C(C)(C)O)ccc3Oc3c(C)cc(F)cc3C)c2s1]

or a pharmaceutically acceptable salt, a tautomer, a solvate, or a deuterated analog thereof.

In some embodiments, said BET inhibitor comprises ABBV-744:

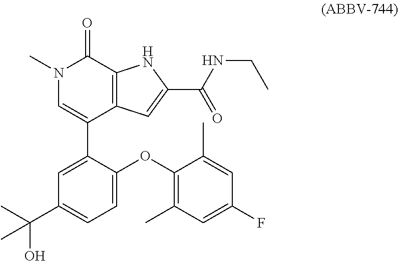 [SMILES: CCNC(=O)c1cc2c(-c3cc(C(C)(C)O)ccc3Oc3c(C)cc(F)cc3C)cn(C)c(=O)c2[nH]1]

or a pharmaceutically acceptable salt, a tautomer, a solvate, or a deuterated analog thereof.

In some embodiments, said cancer comprises bladder cancer, lung cancer, gynecologic cancer, adrenocortical carcinoma, bone cancer, central nervous system (CNS) cancer, pancreatic cancer, gastrointestinal cancer, head and neck cancer, skin cancer, mesothelioma, nerve sheath tumor, lymphoma, renal cell carcinoma, salivary gland cancer, non-melanoma skin cancer, or a combination thereof.

In some embodiments, said cancer comprises lung cancer. In some embodiments, said lung cancer comprises non-small cell lung cancer. In some embodiments, said non-small cell lung cancer comprises squamous cell carcinoma, large cell carcinoma, adenocarcinoma, or a combination thereof. In some embodiments, said non-small cell lung cancer comprises squamous cell carcinoma. In some embodiments, said non-small cell lung cancer comprises large cell carcinoma. In some embodiments, said non-small cell lung cancer comprises adenocarcinoma. In some embodiments, said lung cancer comprises small cell lung cancer. In some embodiments, said cancer comprises breast cancer. In some embodiments, said breast cancer comprises triple-negative breast cancer. In some embodiments, said BET inhibitor comprises AZD-5153, INCB57643, OTX-015, PLX-51107, or a combination thereof. In some embodiments, said BET inhibitor comprises AZD-5153, BI-894999, BMS-986158, GSK525762, INCB054329, OTX-015, PLX-51107, INCB057643, TEN-010, CC-90010, Alobresib, ODM-207, GSK778, GSK046, ABBV-744, Mivebresib, Trotabresib, Molibresib, Birabresib, Pelabresib, or a combination thereof.

In some embodiments, said cancer comprises a squamous cell carcinoma. In some embodiments, said cancer comprises bladder cancer. In some embodiments, said bladder cancer comprises a carcinoma. In some embodiments, said cancer comprises a squamous cell carcinoma. In some embodiments, said carcinoma comprises urothelial carcinoma. In some embodiments, said BET inhibitor comprises AZD-5153, BMS-986158, GSK525762, INCB57643, OTX-015, PLX-51107, or a combination thereof. In some embodiments, said BET inhibitor comprises AZD-5153, BI-894999, BMS-986158, GSK525762, INCB054329, OTX-015, PLX-51107, INCB057643, TEN-010, CC-90010, Alobresib, ODM-207, GSK778, GSK046, ABBV-744, Mivebresib, Trotabresib, Molibresib, Birabresib, Pelabresib, or a combination thereof. In some embodiments, said cancer comprises pancreatic cancer. In some embodiments, said BET inhibitor comprises BI-894999, BMS-986158, GSK525762, INCB054329, INCB57643, OTX-015, PLX-51107, or a combination thereof. In some embodiments, said BET inhibitor comprises AZD-5153, BI-894999, BMS-986158, GSK525762, INCB054329, OTX-015, PLX-51107, INCB057643, TEN-010, CC-90010, Alobresib, ODM-207, GSK778, GSK046, ABBV-744, Mivebresib, Trotabresib, Molibresib, Birabresib, Pelabresib, or a combination thereof.

In some embodiments, said skin cancer comprises melanoma. In some embodiments, said CNS cancer comprises a glioma. In some embodiments, said CNS cancer comprises a neuroepithelial tumor. In some embodiments, said lymphoma comprises Non-Hodgkin lymphoma. In some embodiments, said lymphoma comprises diffuse large B cell lymphoma. In some embodiments, said lymphoma comprises follicular lymphoma. In some embodiments, said lymphoma comprises marginal zone lymphoma. In some embodiments, said lymphoma comprises mature B-cell neoplasm. In some embodiments, said gastrointestinal cancer comprises a gastrointestinal stromal tumor, esophagogastric cancer, gastrointestinal neuroendocrine tumor, small bowel cancer, anal cancer, colon cancer, or a combination thereof.

In some embodiments, said gastrointestinal cancer comprises a gastrointestinal stromal tumor. In some embodiments, said gastrointestinal cancer comprises an esophagogastric cancer. In some embodiments, said gastrointestinal cancer comprises a gastrointestinal neuroendocrine tumor. In some embodiments, said gastrointestinal cancer comprises a small bowel cancer. In some embodiments, said gastrointestinal cancer comprises an anal cancer. In some embodiments, said gastrointestinal cancer comprises colon cancer. In some embodiments, said colon cancer comprises colorectal cancer.

In some embodiments, said gynecologic cancer comprises a cervical cancer. In some embodiments, said gynecologic cancer comprises an ovarian cancer. In some embodiments, said gynecologic cancer comprises a sex cord stromal tumor. In some embodiments, said gynecologic cancer comprises a vaginal cancer. In some embodiments, said cancer comprises uterine cancer. In some embodiments, said uterine cancer comprises endometrial cancer or uterine sarcoma. In some embodiments, said uterine cancer comprises endometrial cancer. In some embodiments, said uterine cancer comprises uterine sarcoma. In some embodiments, said endometrial cancer comprises uterine corpus endometrial carcinoma. In some embodiments, said uterine sarcoma comprises uterine carcinosarcoma. In some embodiments, said BET inhibitor inhibits any one or more of the following bromodomains: BRD2, BRD3, and BRD4. In some embodiments, said BET inhibitor inhibits BRD3 and BRD4. In some embodiments, said BET inhibitor inhibits BRD4 and BRD2. In some embodiments, said BET inhibitor inhibits BD1 or BD2 bromodomains of BRD2, BRD3, and BRD4. In some embodiments, said BET inhibitor comprises a selective BET inhibitor. In some embodiments, said selective BET inhibitor inhibits any one or more of the following bromodomains: BRD2, BRD3, and BRD4. In some embodiments, said selective BET inhibitor is selective for BRD4 and BRD2. In some embodiments, said selective BET inhibitor is selective for BRD2, BRD3, and BRD4. In some embodiments, said selective BET inhibitor selectively inhibits BD1. In some embodiments, said selective BET inhibitor selectively inhibits BD2. In some embodiments, the selective BD2 inhibitor comprises GSK046, ABBV-744, or Compound 9. In some embodiments, the selective BD1 inhibitor comprises GSK778. In some embodiments, said selective BET inhibitor inhibits BD1 or BD2 bromodomains of BRD2, BRD3, and BRD4. In some embodiments, said loss of function or deletion is for an EP300 gene.

In some embodiments, said BET inhibitor comprises JAB-8263. In some embodiments, said BET inhibitor comprises a compound of Formula IV. In some embodiments, said BET inhibitor comprises Compound 5. In some embodiments, said BET inhibitor comprises Compound 9.

In some embodiments, said cancer comprises NUT midline cancer. In some embodiments, said cancer comprises castration resistant prostate cancer. In some embodiments, said cancer comprises a solid cancer or a liquid cancer. In some embodiments, said liquid cancer is selected from myelofibrosis. In some embodiments, said liquid cancer is selected from myeloma and leukemia. In some embodiments, said liquid cancer is selected from myeloma. In some embodiments, said liquid cancer is selected from leukemia. In some embodiments, said leukemia is selected from acute myeloid leukemia (AMIL) and acute lymphocytic leukemia (ALL). In some embodiments, said leukemia is selected from acute myeloid leukemia (AIL). In some embodiments, said leukemia is selected from acute lymphocytic leukemia (ALL).

a. BET Inhibitors

BET proteins may possess two N-terminal bromodomains (e.g., BD1 and BD2), a common structural feature with other BRD proteins, that allow them to interact with acetylated lysine residues on histone. BET proteins may be structurally distinct from other bromodomain-containing proteins by containing a C-terminal extra-terminal (ET) domain. This ET domain may interact with a variety of cellular proteins such as histone-lysine N-methyltransferase (NSD3) and Jumonji domain-containing 6 (JMJD6), whose interactions are implicated in acute myeloid leukemia (AML) and various solid tumors, respectively. The combination of dual BRDs and the protein-interacting ET domain may allow BET proteins to effectively mediate the bimodal tethering of these cancer-associated factors to specific regions of chromatin.

There are four mammalian conserved members of BET proteins, which include BRD containing 2 (BRD2), BRD3, BRD4, and Brdt. While Brdt is predominantly expressed in germ cells, BRD2, BRD3, and BRD4 are ubiquitously expressed in various tissues. All BET proteins adopt a left-handed four-helix bundle structure (αZ, αA, αB, and αC), called the “BRD fold”. The inter-helical αZ-αA (ZA) and αB-αC (BC) loops constitute a hydrophobic pocket that recognizes the acetylated lysine residues. The sequence variations in the ZA and BC loops of the distinct BRD fold in different BET proteins lead to their differential protein-binding sites and affinities. Multiple BET proteins may be required for the rapid induction of selected target genes. Thus, different BET proteins may have non-overlapping functions, and they may form protein complexes with one another to elicit their biological activities.

BET proteins may act as transcription regulators. For example, BRD4 recruits PTEF-b (positive transcription elongation factor which is a multi-protein complex essential for transcription regulation) to sites of active transcription of cell growth-promoting genes such as MYC and NUT. During transcription elongation, BRD4 directs the proper nuclear localization and activation of PTEF-b to phosphorylate RNA polymerase II. Therefore, BRD4 may play a critical role in facilitating the enhancement of basal transcription to active elongation by RNA polymerase II. Meanwhile, the ET domain of BRD4 is known to recruit other transcriptional activators including NSD3, JMJD6, and CHD4 to further promote gene transcription. On the other hand, BRD3 binds specifically to the GATA1 transcription factor and upregulates the expression of GATA1-dependent genes. BRD2 is known to interact with E2F, histone acetyltransferases, and histone deacetylases and recruit them to gene promoters, thereby coupling histone acetylation to transcription in a PTEF-b-independent manner.

The BET family of proteins (e.g., BRD2, BRD3, and BRD4) are of significant clinical interest due to their role in cell cycle regulation, epigenetic sensing, and a range of cancers from oral, breast, prostate, lung, colon, to myeloid leukemia. BET proteins contain dual bromodomains, a domain present in a variety of cellular proteins, which selectively bind acetylated histone marks. These include histone-lysine N-methyltransferase protein ASH1L, histone acetyltransferase p300 (EP300), P300/CBP associated factor (PCAF), and the extended BET family. Current therapeutic approaches rely on small-molecule acetylation mimics which block the ability of the bromodomains to bind their specific chromatin marks. Cancer inhibitors targeting bromodomains have seen limitations in the clinic due to dose-limiting toxicities as they target any proteins containing bromodomains. There has been enormous interest in developing novel BET inhibitors for cancer treatment. A number of the novel candidates are currently progressing through early clinical trials (Targeting BET bromodomains in cancer: Patric Trojer, Annual Review of Cancer Biology, 2022, Vol. 6:313-336; Achieving clinical success with BET inhibitors as anti-cancer agents: Shorstova, British Journal of Cancer, volume 124, pages 1478-1490 (2021)).

In some embodiments, said BET inhibitor comprises ABBV-075, ABBV-744, Apabetalone, APL-581, ARV-825, AZD-5153, BI-6727, BI-894999, BMS-986158, BOS-475, BPI-23314, CD-161, CG-223, CK-103, CN-470, FT-1101, GNE-0011, GS-5829, GS-626510, GSK525762, I-BET151, INCB054329, INCB57643, JQ1LY-294002, NE02734, ODM-207, OMT-001, OMT-002, OTX-015, Pelabresib, PLX-2853 (OPN-2853), PLX-51107 (OPN-51107), TEN-010, RVX-297, SRX-2523, SRX-3225, SRX-3254, SW-064652, SYHA-1801, TTI-281, Zen-3694, CPI-0610, INCB0543294, INCB057643, CC-90010, Alobresib, GSK778, GSK046, Mivebresib, Trotabresib, Molibresib, Pelabresib or a combination thereof. In some embodiments, said BET inhibitor comprises AZD-5153, BI-894999, BMS-986158, GSK525762, INCB054329, INCB57643, OTX-015, PLX-51107, JAB-8263, or a combination thereof. In some embodiments, the BET inhibitor comprises AZD-5153, BI-894999, BMS-986158, GSK525762, INCB054329, OTX-015, PLX-51107, INCB057643, TEN-010, CC-90010, Alobresib, ODM-207, GSK778, GSK046, ABBV-744, Mivebresib, Trotabresib, Molibresib, Birabresib, Pelabresib, or a combination thereof. In some embodiments, said BET inhibitor comprises AZD-5153. In some embodiments, said BET inhibitor comprises BI-894999. In some embodiments, said BET inhibitor comprises BMS-986158. In some embodiments, said BET inhibitor comprises GSK525762. In some embodiments, said BET inhibitor comprises INCB054329. In some embodiments, said BET inhibitor comprises TEN-010. In some embodiments, said BET inhibitor comprises PLX-51107. In some embodiments, said BET inhibitor comprises PLX-2853. In some embodiments, said BET inhibitor comprises OTX-015. In some embodiments, said BET inhibitor comprises JAB-8263. In some embodiments, said BET inhibitor comprises INCB057643. In some embodiments, said BET inhibitor comprises CC-90010. In some embodiments, said BET inhibitor comprises Alobresib. In some embodiments, said BET inhibitor comprises ODM-207. In some embodiments, said BET inhibitor comprises GSK778. In some embodiments, said BET inhibitor comprises GSK046. In some embodiments, said BET inhibitor comprises ABBV-744. In some embodiments, said BET inhibitor comprises Mivebresib. In some embodiments, said BET inhibitor comprises Trotabresib. In some embodiments, said BET inhibitor comprises Molibresib. In some embodiments, said BET inhibitor comprises Birabresib. In some embodiments, said BET inhibitor comprises Pelabresib. In some embodiments, said BET inhibitor comprises a compound of Formula IV. In some embodiments, said BET inhibitor comprises Compound 5. In some embodiments, said BET inhibitor comprises Compound 9. Provided below are non-limiting descriptions of a few exemplary BET inhibitors.

i. JQ1

JQ1 (thieno-triazolo-1,4-diazepine) is a BET inhibitor was designed to mimic acetylated lysine. It has been shown to bind competitively and specifically to the BD1 and BD2 bromodomains with high affinity, forming a hydrogen bond with a conserved asparagine residue at the binding pocket. Using chromatin immunoprecipitation and fluorescence recovery after photobleaching assays, JQ1 was shown to displace BRD4 from chromatin and thus modulate bromodomain-regulated genes. JQ1 exhibited a potent antiproliferative effect in BRD4-dependent cancer cells lines and against NMC. However, JQ1 didn't show good efficacy due to short half-life and rapid metabolism.

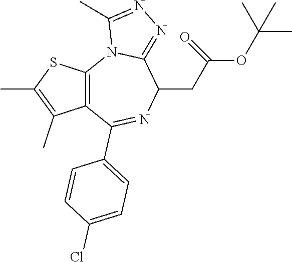 [SMILES: Cc1sc2c(c1C)C(c1ccc(Cl)cc1)=NC(CC(=O)OC(C)(C)C)c1nnc(C)n1-2]

ii. Birabresib

Birabresib, also referred to as OTX-015, is a potent bromodomain inhibitor of BRD2, BRD 3, and BRD4, and has demonstrated IC50s ranging from 92 to 112 nM. Birabresib (500 nM) has demonstrated strong decreases of BRD2, BRD4 and c-MYC and an increase of HEXIM1 proteins. Birabresib treatment at concentrations of 0.1, 1, 5 μM has induced HIV-1 full-length transcripts and viral outgrowth in resting CD4+ T cells from infected individuals receiving suppressive antiretroviral therapy (ART), while exerting minimal toxicity and effects on T cell activation. Birabresib-mediated activation of HIV-1 involves an increase in CDK9 occupancy and RNAP II C-terminal domain (CTD) phosphorylation.

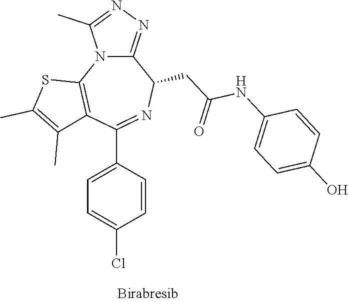 [SMILES: Cc1sc2c(c1C)C(c1ccc(Cl)cc1)=N[C@@H](CC(=O)Nc1ccc(O)cc1)c1nnc(C)n1-2]

iii. PLX-51107

PLX-51107, often referred to as OPN-51107, has been shown to be a potent BET inhibitor, with Kds of 1.6, 2.1, 1.7, and 5 nM for BD1, and Kds of 5.9, 6.2, 6.1, and 120 nM for BD2 of BRD2, BRD3, BRD4, and BRDT, respectively. PLX-51107 also interacts with the bromodomains of CBP and EP300 (Kd, in the 100 nM range). PLX-51107 (0.156-10 MdV) suppresses the CpG-induced proliferation. PLX-51107 may also cause accumulation of p21 and IκBα, reduce c-MYC level, and modulate proapoptotic and antiapoptotic proteins. PLX-51107 selectively modulates CLL driver genes and may be interact with BRD2, BRD3, and BRD4.

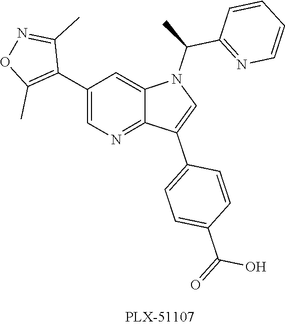 [SMILES: Cc1noc(C)c1-c1cnc2c(-c3ccc(C(=O)O)cc3)cn([C@@H](C)c3ccccn3)c2c1]

iv. PLX-2853

PLX-2853 is an orally available, non-benzodiazepine bromodomain and extraterminal domain (BET) inhibitor that exhibits low nanomolar potency and a modest preference for binding to the second of the double bromodomains of the BET proteins. By regulating genes (e.g., BCL2 and MYC) central to leukemic cell proliferation and survival, PLX-2853 has demonstrated broad anti-leukemic activity both as a single agent and in combination with other agents in preclinical models. The pharmacokinetic (PK) profiles in patients with solid tumors revealed high peak plasma concentrations, a short terminal half-life (T ½<3 hour), and nearly complete elimination from the plasma by 9 hours post dose. This PK profile is hypothesized to improve tolerability by allowing transient target engagement followed by time for recovery after daily dosing. For more information on BET inhibitors, see: U.S. Pat. No. 9,771,363, published on Mar. 23, 2027, entitled “Heterocyclic compounds and uses thereof”, which is incorporated herein in its entirety.

v. JAB-8263

JAB-8263 is a potent BET inhibitor with a subnanomolar binding affinity. Preclinical studies have shown that JAB-8263 can effectively inhibit tumor growth at very low concentrations, including both hematological and solid tumors. It is currently undergoing multi-center, open-label, phase I/IIa clinical studies in China and the United States. For more information on BET inhibitors, such as JAB-8263, see: U.S. Pat. No. 11,466,005, published on Oct. 11, 2022, and US 2021/0179617 A1, published on Jun. 17, 2021, both of which are entitled “Tricyclic Compounds,” and both of which are incorporated herein in their entirety.

b. The Role of EP300 Gene in Cancer

The EP300 gene, also known as E1A Binding Protein P300, is a gene that encodes a protein involved in various cellular processes, including gene regulation, DNA repair, cell growth, and development.

The protein encoded by the EP300 gene is a histone acetyltransferase, meaning it can modify the structure of chromatin (the complex of DNA and proteins in the nucleus) by adding acetyl groups to histone proteins. This modification is crucial for regulating gene expression, as it can make the DNA more accessible to transcription factors and other cellular machinery involved in gene transcription.

In addition to its role in histone acetylation and gene regulation, the p300 protein interacts with a wide range of other proteins, participating in various signaling pathways and molecular interactions within the cell. It plays a critical role in embryonic development, cell differentiation, and response to environmental cues.

Mutations or dysregulation of the EP300 gene have been associated with various diseases and conditions, including certain cancers (such as colorectal and hematologic malignancies), developmental disorders, and neurological conditions.

The P300 protein may play an essential role in regulating cell proliferation and differentiation, and thus, a loss of function mutation or deletion of a EP300 gene may result in a cancer or may exacerbate the severity or metastasis of the cancer. Cancers comprising mutated or deleted EP300 are believed to be present in greater than 4% of all cancers; however, evidence has demonstrated that a loss of function in an EP300 gene is particularly common in bladder cancer, lung cancer, and pancreatic cancer (Table 1). Furthermore, studies have indicated that tumors with EP300 mutation have been associated pathological T stage and lymph node metastasis and have resulted in a shorter predictive cumulative survival status. Accordingly, there is a need for providing treatments to subjects comprising EP300 mutated or deleted cancers.

The methods provided herein may be useful for the treatment of a cancer comprising a EP300 loss of function mutation or a EP300 gene deletion.

In some embodiments, the cancer comprising an EP300 loss of function mutation or EP300 deletion comprises breast cancer, urothelial cancer, rectal cancer, thymus cancer, sarcoma, or a combination thereof. In some embodiments, the cancer comprises bladder cancer, rectal cancer, breast cancer, a sarcoma, thymus cancer, or a combination thereof. In some embodiments, the cancer comprises bladder cancer. In some embodiments, the cancer comprises rectal cancer. In some embodiments, the cancer comprises breast cancer. In some embodiments, the cancer comprises a sarcoma. In some embodiments, the cancer comprises thymus cancer.

In some embodiments, the cancer comprises urothelial cancer. In some embodiments, the urothelial cancer comprises bladder cancer. In some embodiments, the cancer comprises rectal cancer. In some embodiments, the cancer comprises thymus cancer. In some embodiments, the cancer comprises sarcoma.

c. Identification of the Mutation or Deletion of the Gene (e.g., EP300 Gene)

In some embodiments, the cancer has been determined (e.g., previously determined) to comprise the mutation or deletion (e.g., of the EP300 gene). In some embodiments, the methods provided herein comprises determining the presence of a mutation or deletion of the EP300 gene (e.g., in the subject). The presence of the mutation of the gene or deletion of the gene may be determined before administration of the composition. For example, the determination (e.g., previous identification) of the mutation or the deletion may take place months, weeks, days, hours, or even minutes ahead of the administering of the composition (e.g., the composition comprising a BET inhibitor). The determination of the presence of the mutation (e.g., loss of function mutation) in the gene or deletion of the gene (e.g., the EP300 gene) may be conducted in any appropriate manner, including, but not limited to, identification of a gene mutation or gene deletion by any one or more tests discussed herein.

In some embodiments, the mutation or deletion of the EP300 gene is a cause or result of the cancer. In some embodiments, the mutation or deletion of the EP300 gene is not a result of pharmacological inhibition. For example, in such embodiments, the mutation or deletion of the EP300 gene is not caused by administration of a BET inhibitor.

The mutation (e.g., loss of function mutation or deletion in or of a EP300 gene) may be any acceptable type of mutation, such as, for example, a substitution mutation, an insertion mutation, a deletion mutation, or a combination thereof. In some embodiments, the EP300 mutation is a substitution mutation. In some embodiments, the EP300 mutation is an insertion mutation. In some embodiments, the EP300 mutation is a deletion mutation. In some embodiments, the deletion mutation is a partial deletion mutation. In some embodiments, the deletion mutation is a whole deletion mutation. In some embodiments, the mutation may affect one copy of the gene. In some embodiments, the mutation may affect multiple copies of the gene.

Determining if a tumor has a EP300 genetic mutation may include identifying EP300 mutations in DNA extracted from a tumor sample and/or in circulating tumor or tumor cell DNA. In some embodiments, the cancer is determined to comprise the mutation before administration of the BET inhibitor (i.e., the cancer is previously determined as comprising the mutation).

Multiple tests may be employed to detect EP300 mutations or deletions. For example, tests may include sequencing of the tumor DNA using Sanger sequencing of PCR-amplified EP300 encoding regions or next-gen sequencing (NGS) of whole genome or captured/enriched EP300 encoding regions (e.g., through whole-exome sequencing, or sequencing of a targeted mutation panel that includes EP300 exons and introns).

Mutations may also be detected in RNA. Such RNA mutations may be detected using RNA-Seq or DNA approaches to sequence cDNA derived from RNA. Furthermore, mutations may be detected through targeted amplification of variants and sequenced by next-generation sequencing (NGS) or through array-based readout of genetic variants (e.g. using Illumina BeadArrays). Tumor DNA may be derived from biopsy samples or captured from indirect tumor sources, such as blood derived cell-free DNA.

Function and amount of EP300 protein may be determined through any acceptable method, including, for example, methods that measure the acetylation levels of proteins known to be modified by p300. Methods to quantify these modifications may include western blots, mass-spectrometry, protein binding arrays, or immunohistochemistry.

d. Improving Cancer Treatment Efficacy

Methods provided herein may be useful in treating a cancer, such as, for example, by increasing cancer cell inhibition or decreasing cancer progression. “Increases cancer cell inhibition” may mean decreasing cancer cell viability or inhibiting cancer cell growth or proliferation.

In some embodiments, the administering of a BET inhibitor increases cancer cell inhibition. In some embodiments, the administering of a BET inhibitor increases cancer cell inhibition as compared to a different treatment. In some embodiments, administering of a BET inhibitor increases cancer cell inhibition in a cancer comprising a loss of function or a deletion of a EP300 gene compared to a corresponding administration of said BET inhibitor to a cancer that does not have a loss of function or a deletion.

e. Administration

Administration of the composition comprising a BET inhibitor may be accomplished by any acceptable means, including, for example, by parenteral administration. Acceptable means of parenteral administration include, but are not limited to, subcutaneous administration, intramuscular administration, and intravenous administration. In some embodiments, the method comprises administering a BET inhibitor. In some embodiments, administering comprises administering the BET inhibitor parenterally. In some embodiments, the method comprises administering a pharmaceutical composition comprising a BET inhibitor (e.g., such as any of the pharmaceutical compositions provided herein).

In some embodiments, administering comprises administering the BET inhibitor enterally. Acceptable means of enteral administration include, but are not limited to, oral administration, gastric administration, and rectal administration.

In some embodiments, administering occurs once daily. In some embodiments, administering occurs twice daily. In some embodiments, administering occurs three times daily.

f. Pharmaceutical Compositions

The BET inhibitor (e.g., PLX-51107) may be delivered in the form of a pharmaceutical composition. In some embodiments, the pharmaceutical composition comprises a BET inhibitor. The pharmaceutical composition may be useful in the treatment of a cancer, such as, for example, any of the cancers disclosed herein (e.g., a cancer determined as comprising a loss of function or a deletion of a EP300 gene). In some embodiments, the methods provided herein comprises determining the presence of a mutation or deletion of the EP300 gene (e.g., in the subject).

The compositions described herein may comprise any appropriate BET inhibitor, such as, for example, any of the BET inhibitors described herein. In some embodiments, the BET inhibitor is a stereoisomer, pharmaceutically acceptable salt, hydrate, or solvate of a BET inhibitor described herein. In some embodiments, the pharmaceutical composition comprises a BET inhibitor and a pharmaceutically acceptable carrier (e.g., one or more pharmaceutically acceptable carrier, two or more pharmaceutically acceptable carriers, three or more pharmaceutically acceptable carriers, etc.). The carrier(s) may be any acceptable or suitable described herein. In some embodiments, the pharmaceutical composition comprising a BET inhibitor is administered in a method of treating a patient (e.g., comprising a EP300 loss of function or a deletion of EP300).

In certain embodiments, the BET inhibitor described herein is administered as a pure chemical (i.e., not with an excipient). In some embodiments, the BET inhibitor described herein is combined with a pharmaceutically suitable or acceptable carrier (which may also be referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

In some embodiments, the pharmaceutical composition comprising a BET inhibitor is formulated for oral administration. Suitable oral dosage forms include, but are not limited to, tablets, pills, sachets, or capsules.

In some embodiments, the pharmaceutical composition comprising a BET inhibitor is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising a BET inhibitor may differ depending upon the subject or patient's condition. Such factors for consideration may include general health status, age, and other factors.

The pharmaceutical compositions described herein may be administered in any manner appropriate for the treatment or prevention of the disease (e.g., cancer). An appropriate dose and a suitable duration and frequency of administration may be determined by factors related to the condition of the patient, such as the type and severity of the patient's disease, the patient's age, weight, body surface area, etc. Alternatively, an appropriate dose and a suitable duration and frequency of administration may be determined by factors related to the composition, such as the particular form of the active ingredient, and the method of administration. In cases, the appropriate dose is determined by factors related to both the condition of the patient and the composition. In general, an appropriate dose and treatment regimen may provide the composition(s) in an amount sufficient to provide therapeutic and/or prophylactic benefit (e.g., an improved clinical outcome, such as more frequent complete or partial remissions, or longer disease-free and/or overall survival). Optimal doses are generally determined using experimental models and/or clinical trials. The optimal dose may depend upon the body mass, weight, or blood volume of the patient.

### II. Kits for Treating Cancer

Further provided herein, in some embodiments, are kits for treating a cancer, comprising: a test for determining a loss of function or a deletion of a EP300 gene. In some embodiments, said BET inhibitor comprises ABBV-075, ABBV-744, Apabetalone, APL-581, ARV-825, AZD-5153, BI-6727, BI-894999, BMS-986158, BOS-475, BPI-23314, CD-161, CG-223, CK-103, CN-470, FT-1101, GNE-0011, GS-5829, GS-626510, GSK525762, I-BET151, INCB054329, INCB57643, JQ1LY-294002, NE02734, ODM-207, OMT-001, OMT-002, OTX-015, Pelabresib, PLX-2853 (OPN-2853), PLX-51107 (OPN-51107), TEN-010, RVX-297, SRX-2523, SRX-3225, SRX-3254, SW-064652, SYHA-1801, TTI-281, Zen-3694, CPI-0610, INCB0543294, JAB-8263, INCB057643, CC-90010, Alobresib, GSK778, GSK046, Mivebresib, Trotabresib, Molibresib, Pelabresib, or a combination thereof. In some embodiments, said BET inhibitor comprises AZD-5153, BI-894999, BMS-986158, GSK525762, INCB054329, INCB57643, OTX-015, PLX-51107, or a combination thereof. In some embodiments, said BET inhibitor comprises AZD-5153, BI-894999, BMS-986158, GSK525762, INCB054329, OTX-015, PLX-51107, INCB057643, TEN-010, CC-90010, Alobresib, ODM-207, GSK778, GSK046, ABBV-744, Mivebresib, Trotabresib, Molibresib, Birabresib, Pelabresib, or a combination thereof. In some embodiments, said BET inhibitor is OTX-015, PLX-51107, PLX-2853, or a combination thereof. In some embodiments, said BET inhibitor is JAB-8263. In some embodiments, said BET inhibitor comprises AZD-5153. In some embodiments, said BET inhibitor comprises BI-894999. In some embodiments, said BET inhibitor comprises BMS-986158. In some embodiments, said BET inhibitor comprises GSK525762. In some embodiments, said BET inhibitor comprises INCB054329. In some embodiments, said BET inhibitor comprises TEN-010. In some embodiments, said BET inhibitor comprises PLX-51107. In some embodiments, said BET inhibitor comprises PLX-2853. In some embodiments, said BET inhibitor comprises OTX-015. In some embodiments, said BET inhibitor comprises JAB-8263. In some embodiments, said BET inhibitor comprises INCB057643. In some embodiments, said BET inhibitor comprises CC-90010. In some embodiments, said BET inhibitor comprises Alobresib. In some embodiments, said BET inhibitor comprises ODM-207. In some embodiments, said BET inhibitor comprises GSK778. In some embodiments, said BET inhibitor comprises GSK046. In some embodiments, said BET inhibitor comprises ABBV-744. In some embodiments, said BET inhibitor comprises Mivebresib. In some embodiments, said BET inhibitor comprises Trotabresib. In some embodiments, said BET inhibitor comprises Molibresib. In some embodiments, said BET inhibitor comprises Birabresib. In some embodiments, said BET inhibitor comprises Pelabresib. In some embodiments, said BET inhibitor comprises a compound of Formula IV. In some embodiments, said BET inhibitor comprises Compound 5. In some embodiments, said BET inhibitor comprises Compound 9.

In some embodiments, said cancer comprises breast cancer, urothelial cancer, rectal cancer, thymus cancer, sarcoma, or a combination thereof. In some embodiments, said urothelial cancer comprises bladder cancer. In some embodiments, the methods provided herein comprises determining the presence of a mutation or deletion of the EP300 gene (e.g., in the subject).

### Definitions

As used in the specification and appended claims, unless specified to the contrary, the following terms have the meaning indicated below.

The singular forms (e.g., “a,” “and,” and “the”) include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range, in some instances, the term “about” preceding a stated value includes the stated value and also includes ±20% of the stated value or range, and includes more specifically values of ±10%, ±5%, ±2%, and ±1% of the stated value or range.

The term “associated” or “associated with” in the context of a disease, substance or substance activity or function associated with a disease means that the disease is caused by (in whole or in part), a symptom of the disease is caused by (in whole or in p art) the substance or substance activity or function, or a side-effect of the compound (e.g. toxicity) is caused by (in whole or in part) the substance or substance activity or function.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

“Disease” or “condition” refer to a state of being or health status of a patient or subject capable of being treated with the compounds or methods provided herein. In some embodiments, the disease as used herein refers to cancer.

As defined herein, the terms “inhibition”, “inhibit”, “inhibiting” and the like, when made in reference to a protein-inhibitor interaction, means negatively affecting (e.g. decreasing) the activity or function of the protein (e.g., BET) relative to the activity or function of the protein in the absence of the inhibitor. Thus, inhibition includes, at least in part, partially or totally blocking stimulation, decreasing, preventing, or delaying activation, or inactivating, desensitizing, or down-regulating signal transduction or enzymatic activity or the amount of a protein. Inhibition, and such terms, may refer to reduction of a disease or symptoms of disease when referred to in such context.

The compounds disclosed herein, in some embodiments, contain one or more asymmetric centers and thus give rise to enantiomers, diastereomers, and other stereoisomeric forms that are defined, in terms of absolute stereochemistry, as (R) or (S). Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

As used herein, “delaying development of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

The terms “selective” or “selectivity” “specific”, “specifically”, “specificity”, or the like of a compound refers to the compound's ability to cause a particular action or the compound's ability to discriminate between molecular targets, such as inhibition, to a particular molecular target (e.g., BD2). For example, a selective BD2 inhibitor may have at least a 3× higher selectivity for the BD2 compared to BD1 in a cell. As a further example, a selective BET inhibitor may have at least a 3× higher selectivity for the BET compared to other targets (e.g., Plk1) in a cell.

As used herein, compounds providing inhibitory properties (e.g., a BET inhibitor), include small-molecule compounds and biological products (e.g., such as those derived from living material (e.g., antibodies, proteins, peptide fragments, etc), unless context clearly dictates otherwise.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

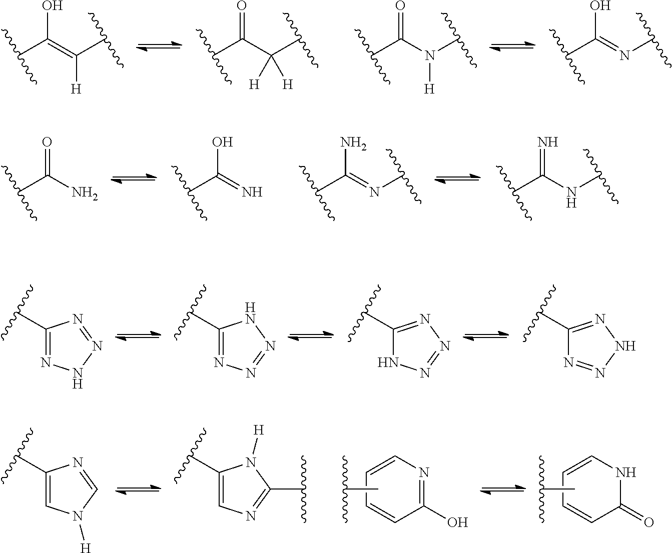

“Pharmaceutically acceptable salt” includes both acid and base addition salts. A pharmaceutically acceptable salt of any one of the BET inhibitors described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein exist in either unsolvated or solvated forms.

The term “subject”, “individual” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “mutations” may refer to changes in a polynucleotide sequence that result in changes to protein function. A change to protein function may occur through a change to the protein encoded by the polynucleotide sequence. The change in function of the protein may be altered through an amino acid sequence of the protein or by a change in the amount (e.g., in an expression level) of the protein, or both. Mutations can be nucleotide substitutions, such as single nucleotide substitutions, insertions, or deletions, and may end up altering the splicing of messenger RNA (mRNA), the levels of mRNA, and/or the amino acid sequence of the protein encoded by said mRNA. Mutations may also occur in regions that regulate the expression levels or processing of the protein.

As used herein, “loss-of-function” can arise through 1) an amino acid sequence change that leads to loss of or reduction in one of more normal functions of the protein or, 2) reduction of the EP300 protein amount. These changes can be caused by genetic alterations (changes to genomic DNA), changes to normal mRNA production and processing (e.g. through a splice alteration or silencing of gene expression through a gene expression regulatory alteration such as a mutation in the regulatory regions of the gene or by epigenetic silencing), and/or an alteration in protein translation that leads to incorrect protein sequence synthesis or aberrant protein folding.

As used herein, “treatment” or “treating,” or “palliating” or “ameliorating” are used interchangeably. These terms refer to an approach for obtaining beneficial or desired results, including, but not limited to, a therapeutic benefit and/or a prophylactic benefit. The term “therapeutic benefit”, or the like, may include eradication or amelioration of the underlying disorder being treated. Also, a therapeutic benefit may be achieved with the eradication or amelioration of one or more of the symptoms associated with the underlying disorder such that an improvement may be observed in the patient, notwithstanding that the patient may still afflicted with the underlying disorder. For achieving a prophylactic benefit, the compositions may be administered to a patient at risk of developing a particular disease, or to a patient reporting one or more of the physiological symptoms of a disease, even though a diagnosis of this disease has not been made.

### EXAMPLES

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. Itis intended that the following claims define the scope of the invention and that methods and structures within the scope of these claims and their equivalents be covered thereby.

### Example 1—Identification of Mutations in EP300 Gene

Experiments were run in order to identify potential loss of function mutations and deletions in EP300. Genomic DNA was extracted from FFPE-preserved tumor biopsy tissue. Targeted capture was used to enrich for the exome, including all coding exons. NGS libraries were constructed and indexed using Illumina TruSeq exome kits (cat number 20020614) and then sequenced on an Illumina HiSeq platform. Somatic mutations were then called (e.g. per methods described in PMID 24192750 or PMID 33106175). Genome variants (SNVs and indels) were identified using GATK (PMID 20644199), and somatic mutations were called using Mutect2 (PMID 23396013) and Varscan (PMID 19542151). The mutations were filtered to functional/deleterious mutations on the basis of causing amino acid substitutions or protein coding changes with a predicted functional effect inferred by MutationTaster (PMID 20676075), a “deleterious” flag inferred by SIFT (PMID 12824425), and/or a “probably damaging” flag inferred by Polyphen-2 (PMID 23315928). Loss of function (LOF) mutations may include hotspot mutations.

Shallow (e.g. single copy loss) and deep deletions (e.g., which may include a complete (zero copy)) loss of one or more regions of EP300 that overlap a portion of a coding exon or an entire coding exon) may also constitute EP300 LOF events. Deletions and a complete loss of the EP300 gene were called from exome data using Control-FREEC (PMID 22155870). Western analysis and immunohistochemistry on FFPE preserved tumor biopsy was used to confirm a reduction or loss of EP300 gene expression at the protein level.

### Example 2—Frequency of EP300 Alterations in Various Cancers

Estimated frequencies of some EP300 loss of function events across cancers from Project GENIE (Genomics Evidence Neoplasia Information Exchange) are presented in the below Table 1.

TABLE 1

| | | Deep Deletion | |
|---|---|---|---|
| | Mutation | Frequencies | Deletion |
| Cancer Type | Frequency | (0 copies) | (1 copy) |
| Anal Cancer | 11.93% | 0.00% | 3.29% |
| Bladder Cancer | 11.23% | 0.08% | 2.82% |
| Skin Cancer, Non-Melanoma | 10.27% | 0.14% | 2.25% |
| Head and Neck Cancer | 7.80% | 0.00% | 1.59% |
| Vaginal Cancer | 6.96% | 0.00% | 1.74% |
| Endometrial Cancer | 6.95% | 0.03% | 3.54% |
| Small Bowel Cancer | 6.67% | 0.00% | 4.00% |
| Non-Hodgkin Lymphoma | 5.88% | 0.00% | 0.00% |
| Colorectal Cancer | 4.59% | 0.02% | 5.46% |
| Melanoma | 4.37% | 0.00% | 0.86% |
| Cervical Cancer | 4.29% | 0.19% | 3.12% |
| Mature B-Cell Neoplasms | 3.93% | 0.10% | 0.29% |
| Small Cell Lung Cancer | 3.73% | 0.30% | 7.01% |
| Esophagogastric Cancer | 3.46% | 0.00% | 3.32% |
| Non-Small Cell Lung Cancer | 2.86% | 0.01% | 2.81% |
| Renal Cell Carcinoma | 2.59% | 0.00% | 2.15% |
| Glioma | 2.20% | 0.17% | 8.98% |
| Ovarian Cancer | 2.10% | 0.38% | 10.96% |
| Breast Cancer | 1.93% | 0.06% | 10.86% |
| Uterine Sarcoma | 1.67% | 0.21% | 8.56% |
| Adrenocortical Carcinoma | 0.83% | 4.96% | 14.88% |
| Gastrointestinal Stromal Tumor | 0.48% | 0.12% | 22.25% |

### Example 3. Cellular Assay

To study the effect of BET inhibitors on cells with a EP300 LOF mutation, cells with and without EP300 LOF mutations were treated with a BET inhibitor.

Isogenic pairs of cancer cell lines differing only in their EP300 mutation status (with and without EP300 loss of function) were generated. Synthego CRISPR Gene Knockout v2 kits were used to knock out EP300 in KP4 cells (where EP300 is wild type and present in 2 copies; PMID 23550210). The knockout of EP300 was confirmed by Sanger sequencing and Synthego analysis software. Cells in 96-well plates at 2000 cells/well were seeded in triplicate and treated with varying concentrations of BET inhibitors spanning a 3× dilution gradient from 50 uM to sub-nanomolar concentrations. 4-11 days after treatment initiation, cell viability was determined by cell titer glo.

The curves revealed a dose-dependent response to BET inhibitors, with higher concentrations leading to greater reductions in cell viability, and overall higher sensitivity in cells with EP300 knocked out (i.e. a lower concentration of drug would yield the same reduction of viability in EP300-LOF vs EP300 wt).

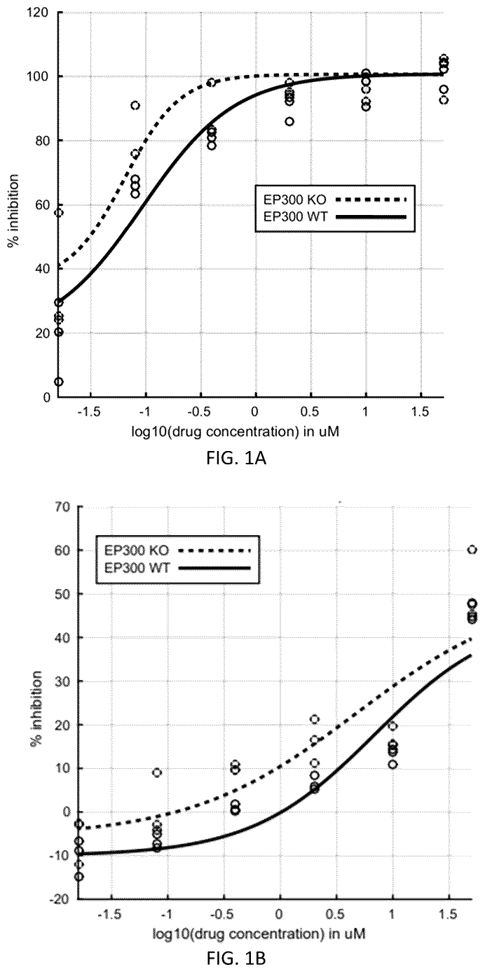

FIGS. 1-9, demonstrate the inhibitive effects of various compounds in different cell lines having wild type EP300 or EP300 knocked out. As shown by these figures, cell lines having an EP300 LOF mutation exhibited increased sensitivity to multiple BET inhibitors. FIGS. 1A-1B depict the effect of AZD-5153 in cancer cells with wild type EP300 and EP300 knocked out. FIG. 1A depicts the effect of AZD-5153 in H2009 cells, a lung cancer cell line, whereas FIG. 1B depicts the effect of AZD-5153 in SW780 cells, a bladder cancer cell line.

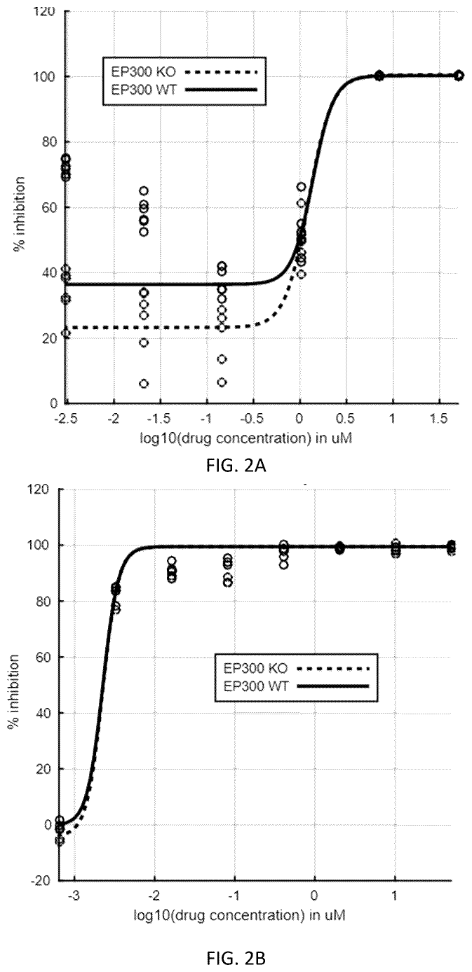

FIGS. 2A-2B depict the effect of BI-2536 in cancer cells with wild type EP300 and EP300 knocked out. FIG. 2A depicts the effect of BI-2536 in SW780 cells, a bladder cancer cell line, whereas FIG. 2B depicts the effect of BI-2536 in KP4 cells, a pancreatic cancer cell line.

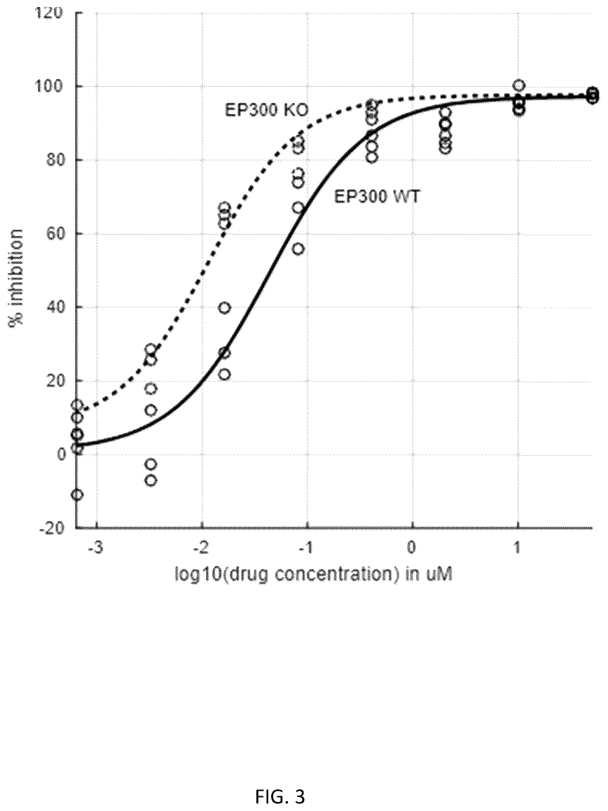

FIG. 3 depicts the effect of BI-894999 in KP4 pancreatic cancer cells with wild type EP300 and EP300 knocked out.

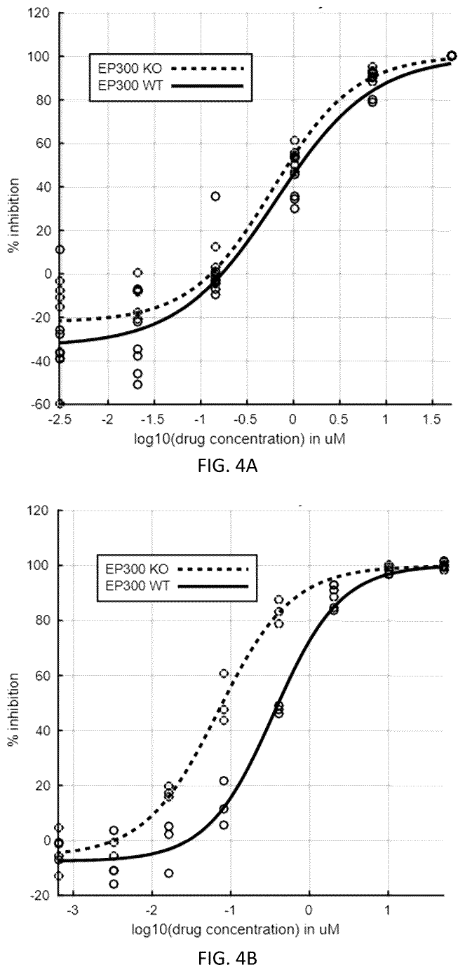

FIGS. 4A-4B depict the effect of BMS-986158 in cancer cells with wild type EP300 and EP300 knocked out. FIG. 4A depicts the effect of BMS-986158 in SW780 cells, a bladder cancer cell line, whereas FIG. 4B depicts the effect of BMS-986158 in KP4 cells, a pancreatic cancer cell line.

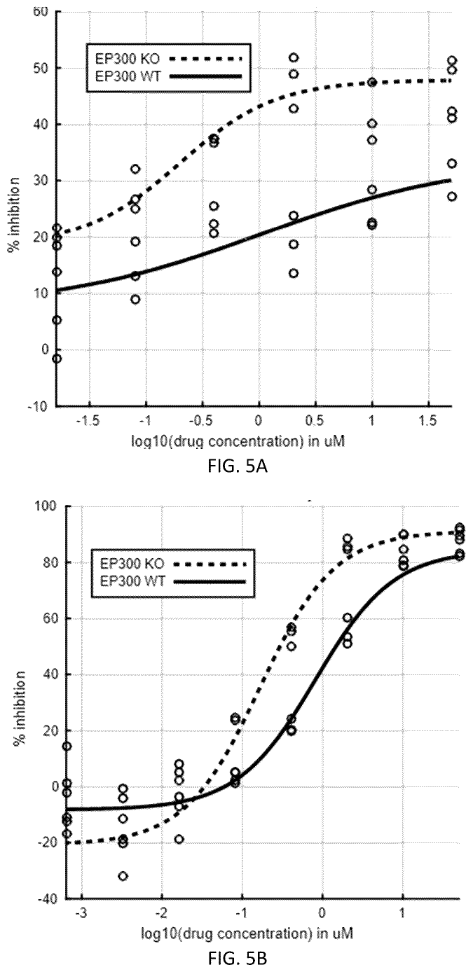

FIGS. 5A-5B depict the effect of GSK525762 in cancer cells with wild type EP300 and EP300 knocked out. FIG. 5A depicts the effect of GSK525762 in SW780 cells, a bladder cancer cell line, whereas FIG. 5B depicts the effect of GSK525762 in KP4 cells, a pancreatic cancer cell line.

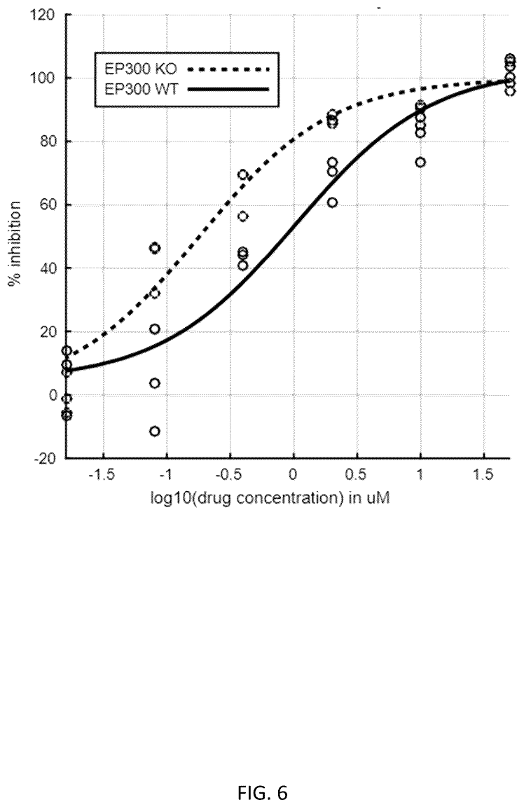

FIG. 6 depicts the effect of INCB054329 in KP4 pancreatic cancer cells with wild type EP300 and EP300 knocked out.

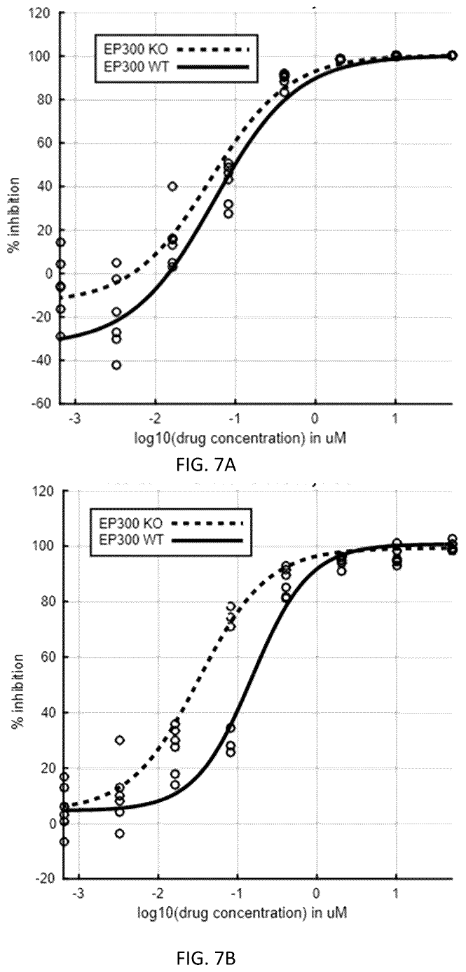

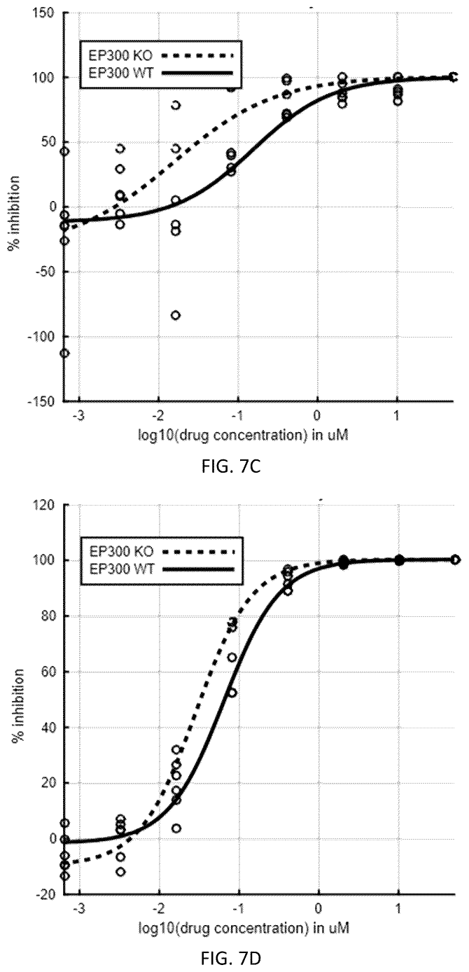

FIGS. 7A-7D depict the effect of OTX-015 in cancer cells with wild type EP300 and EP300 knocked out. FIG. 7A depicts the effect of OTX-015 in RT11284 cells, a bladder cancer cell line. FIG. 7B depicts the effect of OTX-015 in KP4 cells, a pancreatic cancer cell line. FIG. 7C depicts the effect of OTX-015 in SW780 cells, a bladder cancer cell line. FIG. 7D depicts the effect of OTX-015 in Calu1 cells, a lung cancer cell line.

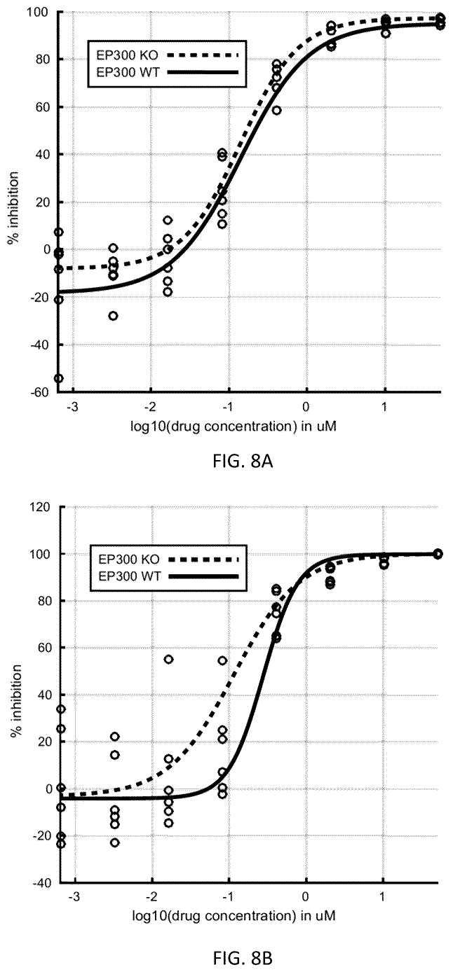

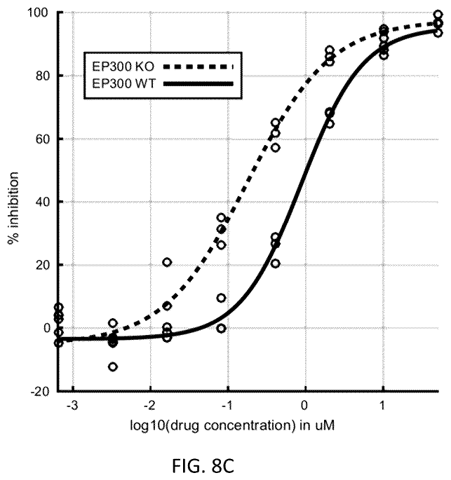

FIGS. 8A-8C depict the effect of PLX-51107 in cancer cells with wild type EP300 and EP300 knocked out. FIG. 8A depicts the effect of PLX-51107 in RT11284 cells, a bladder cancer cell line. FIG. 8B depicts the effect of PLX-51107 in Calu1 cells, a lung cancer cell line. FIG. 8C depicts the effect of PLX-51107 in KP4 cells, a pancreatic cancer cell line.

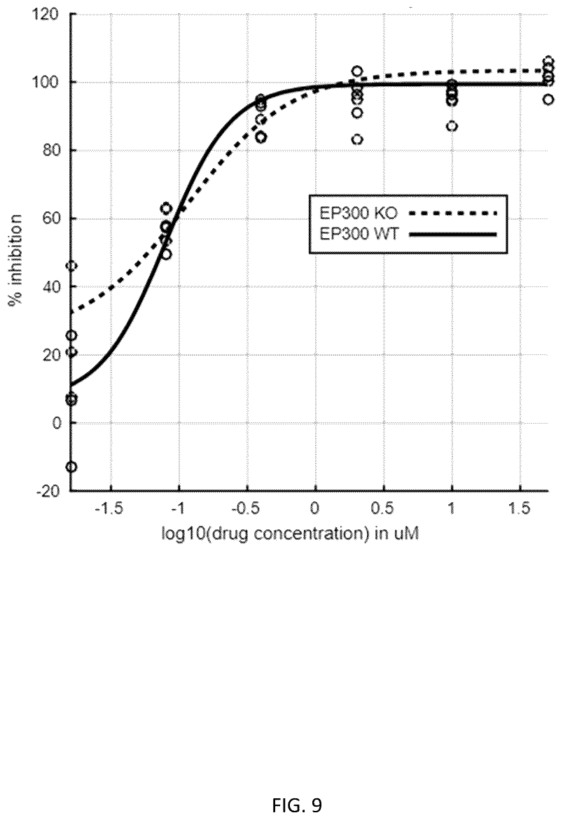

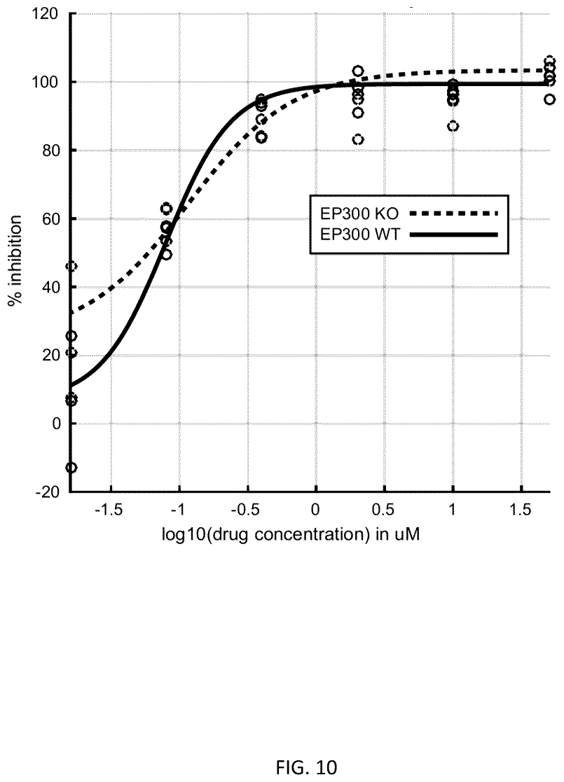

FIG. 9 depicts the effect of TEN-010 in KP4 pancreatic cancer cells with wild type EP300 and EP300 knocked out. FIG. 10 depicts the effect of JQ1 in KP4 pancreatic cells with wild type EP300 and EP300 knocked out.

The results of this experiment demonstrate that there is synthetic lethality between BET inhibitors and an EP300 loss of function mutation for drugs whose primary targets include BRD2, BRD3, and/or BRD4.

### Example 4. Xenograft Study Using Patient Derived Xenograft Models

To investigate the potential for using BET inhibitors (e.g., PLX-51107 and Birabresib) to treat tumors with EP300-LOF, patient derived xenograft (PDX) models with predicted LOF mutations in EP300 were identified from the NCI Patient Derived Model Repository (PDMR) and at Jackson Labs (JAX). Analysis of whole exome sequencing data to identify LOF EP300 mutations were done using methods, such as those described in Example 1. GATK and Samtools were used to call likely somatic single nucleotide variants (SNVs) and indels, which were then filtered on high likelihood of inducing a protein functional change, and further distinguished from gain of function (GOF) mutations on not being in sequence or structural hotspot regions.

PDX samples harboring a EP300 LOF mutation, selected using the criteria described above, were cut to a uniform size and implanted subcutaneously into bilateral flanks of 5-week old NSG mice sourced from JAX. For each study (i.e. each model), a 3×8 (3 treatments, 8 replicates) experimental design is employed. Tumors were measured every other day until tumor volume reached approximately 150 mm³. BET inhibitor is administered (1-20 mg/kg) once daily by oral gavage. Tumor sizes were recorded daily, and body weights were measured every 7 days to monitor for drug toxicity. Tumor volume was calculated using the following formula: tumor volume (mm3)=½ (W)2×(L). Mice were sacrificed once tumors reached 500 mm³. Differential outcomes were determined through a log rank Kaplan-Meier survival analysis or by comparing growth kinetics over time.

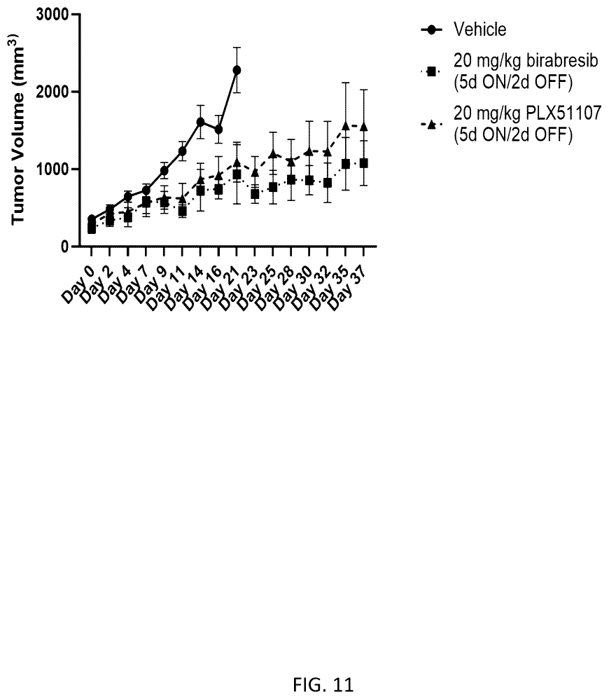

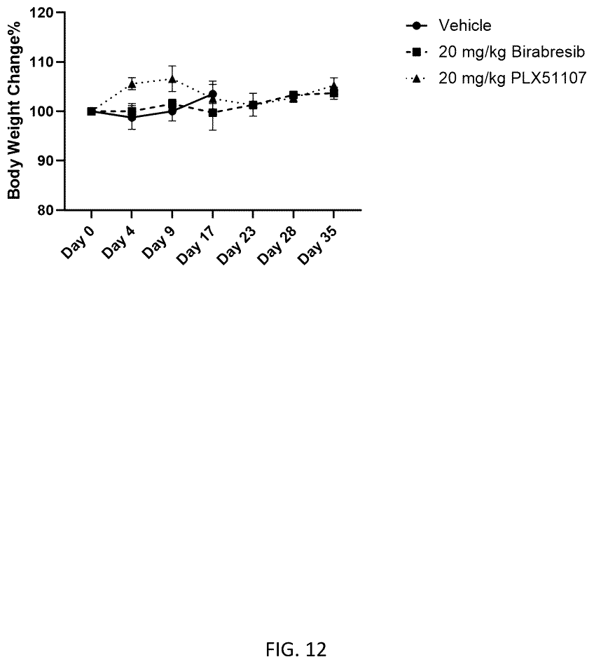

Mice harboring an EP300-LOF mutant PDX model were treated orally with either PLX-51107 or Birabresib at 20 mg/kg achieved significantly reduced tumor growth (FIG. 11). No significant body weight change is observed in mice treated with PLX-51107 or Birabresib (FIG. 12).

### Example 5. Xenograft Study of Compounds of Formula VII (e.g., Compound 9) Using Patient Derived Xenograft Models

To investigate the potential for using compounds of Formula VII (e.g., Compound 9) to treat tumors with EP300-LOF, the techniques presented in Example 4 (“Xenograft Study Using Patient Derived Xenograft Models”) will be utilized with compounds of Formula VII (e.g., Compound 9) at concentrations that model clinically meaningful exposure levels.

Mice harboring an EP300-LOF mutant PDX model will be treated orally with compounds of Formula VII (e.g., Compound 9). Treated mice will achieve significantly reduced tumor growth and with insignificant reductions in body weight.

### Example 6. Patient Selection

Tumor derived FFPE biopsy samples are used to identify subjects with a LOF of EP300. Next-generation sequencing (NGS) of a targeted gene panel that includes all exons of EP300 are used to generate sequencing data. Mutations are called using methods such as those as described in Example 1.

### Example 7. Cellular Assay

To study the effect of BET inhibitors on cells with a EP300 LOF mutation, cells with and without EP300 LOF mutations were treated with a BET inhibitor.

Isogenic pairs of cancer cell lines differing only in their EP300 mutation status (with and without EP300 loss of function) are generated. Synthego CRISPR Gene Knockout v2 kits are used to knock out EP300 in KP4 cells (where EP300 is wild type and present in 2 copies; PMID 23550210). The knockout of EP300 is confirmed by Sanger sequencing and Synthego analysis software. Cells in 96-well plates at 2000 cells/well are seeded in triplicate and treated with varying concentrations of BET inhibitors spanning a 3× dilution gradient from 50 uM to sub-nanomolar concentrations. 4-11 days after treatment initiation, cell viability is determined by cell titer glo.

The curves will reveal a dose-dependent response to BET inhibitors, with higher concentrations leading to greater reductions in cell viability, and overall higher sensitivity in cells with EP300 knocked out (i.e. a lower concentration of drug would yield the same reduction of viability in EP300-LOF vs EP300 wt).

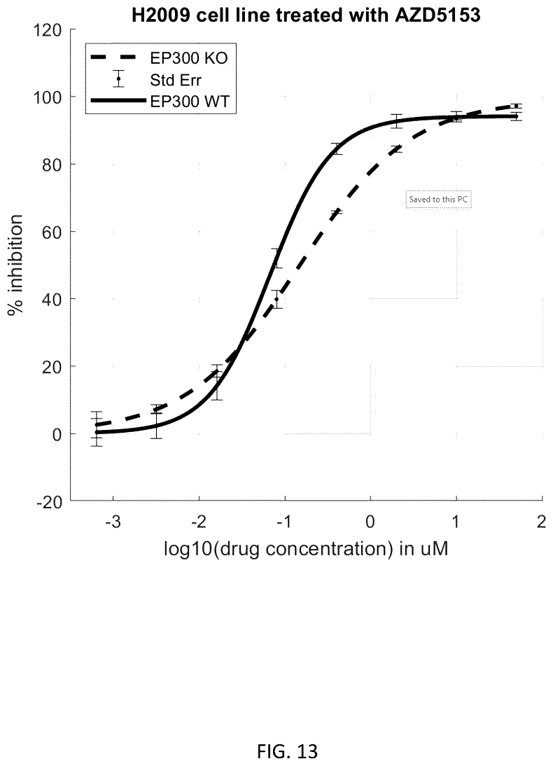

FIGS. 13-44, demonstrate the inhibitive effects of various compounds in different cell lines having wild type EP300 or EP300 knocked out. As shown by these figures, cell lines having an EP300 LOF mutation exhibited increased sensitivity to multiple BET inhibitors.

FIG. 13 depicts the effect of AZD-5153 in H2009 cells, a lung cancer cell line, with wild type EP300 and EP300 knocked out.

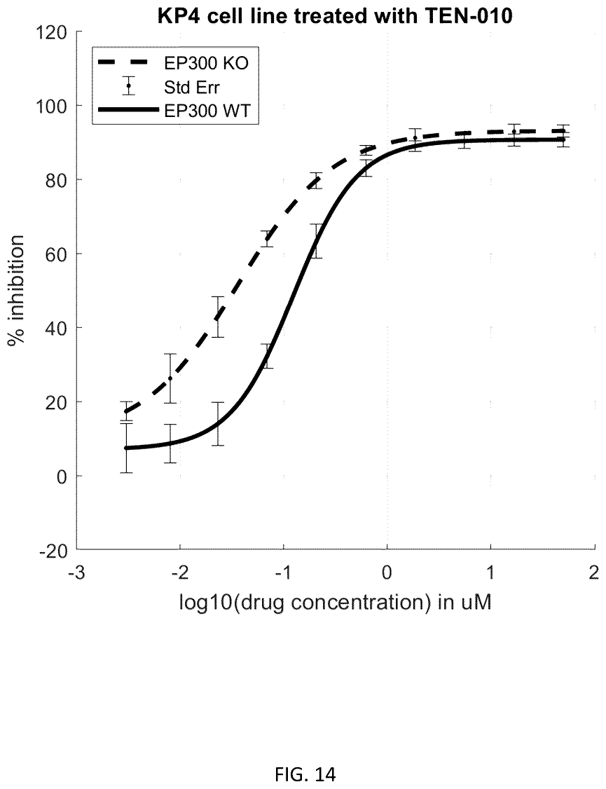

FIG. 14 depicts the effect of TEN-010 in KP4 pancreatic cancer cells with wild type EP300 and EP300 knocked out.

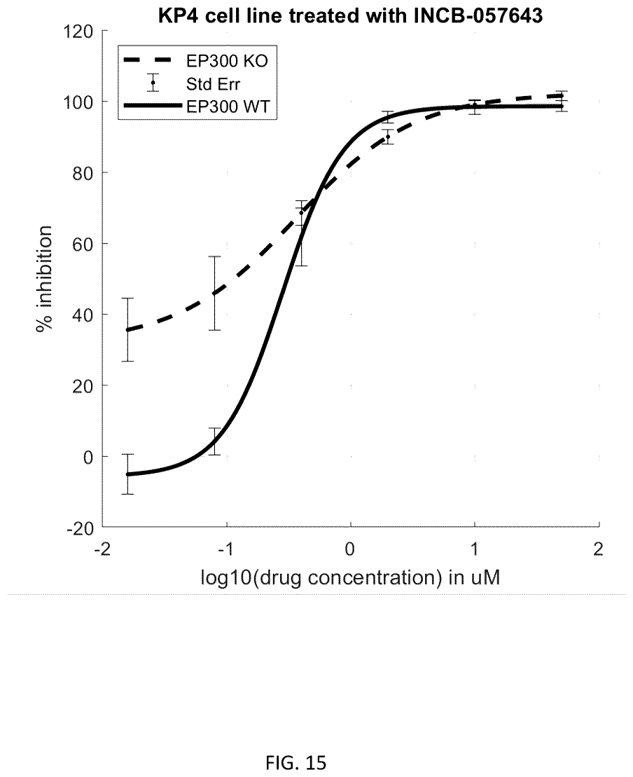

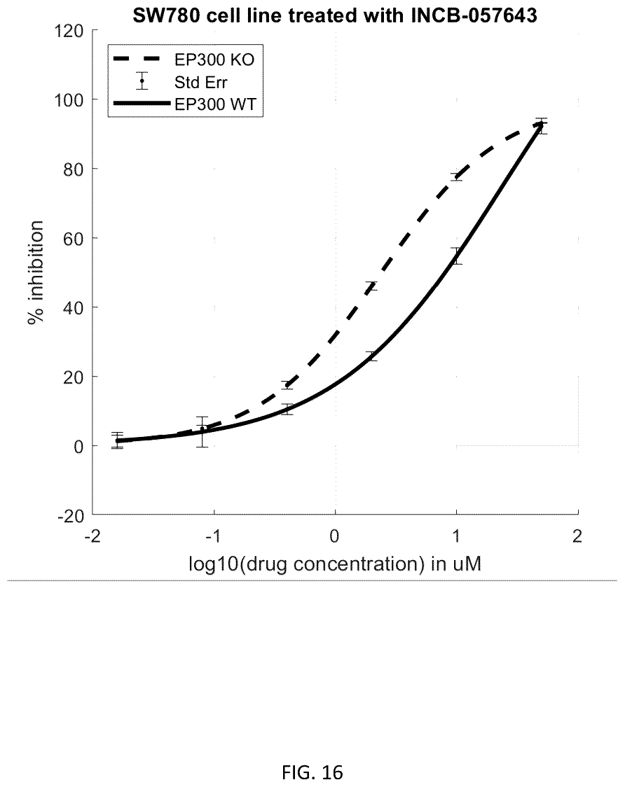

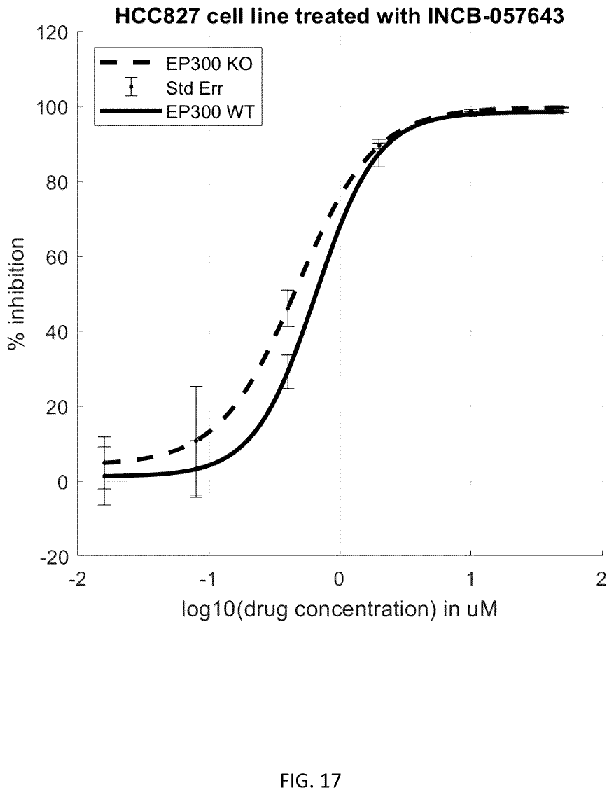

FIGS. 15-17 depict the effect of INCB057643 in cancer cells with wild type EP300 and EP300 knocked out. FIG. 15 depicts the effect of INCB057643 in KP4 pancreatic cancer cells with wild type EP300 and EP300 knocked out. FIG. 16 depicts the effect of INCB057643 in SW780 bladder cancer cells with wild type EP300 and EP300 knocked out. FIG. 17 depicts the effect of INCB057643 in HCC827 lung cancer cells with wild type EP300 and EP300 knocked out.

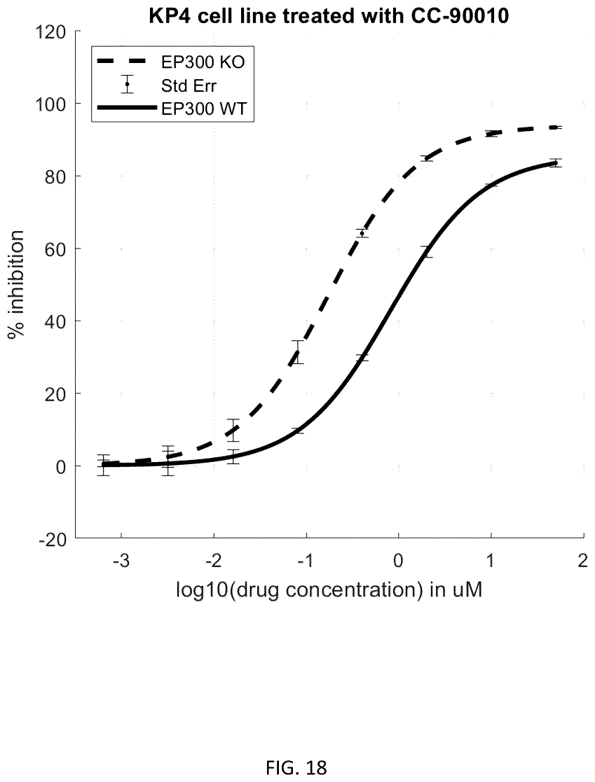

FIG. 18 depicts the effect of CC-90010 in KP4 pancreatic cancer cells with wild type EP300 and EP300 knocked out.

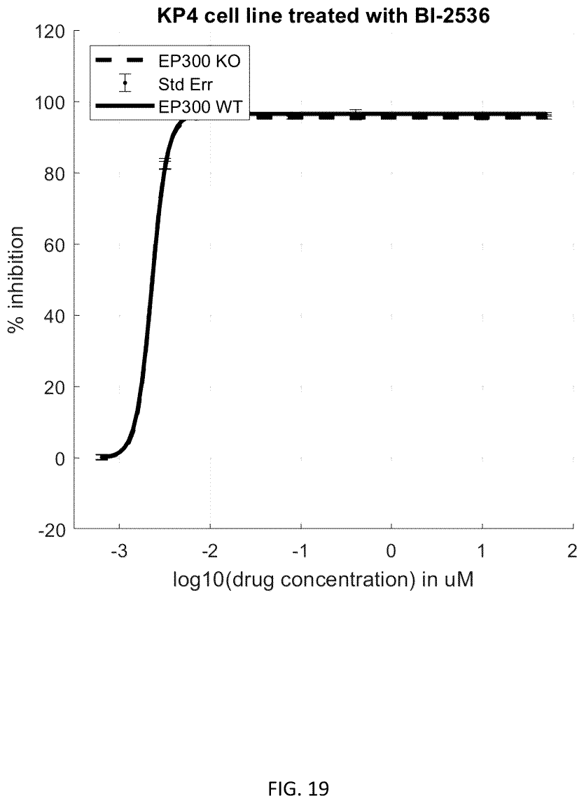

FIG. 19 depicts the effect of BI-2536 in KP4 pancreatic cancer cells with wild type EP300 and EP300 knocked out. BI-2536 is a potent Plk1 inhibitor with >40× selectivity for Plk1 versus BRD4. BI-2536 was included as a negative control and demonstrates the effectiveness of BET inhibitors for synthetic lethality as compared to a Plk1 inhibitor.

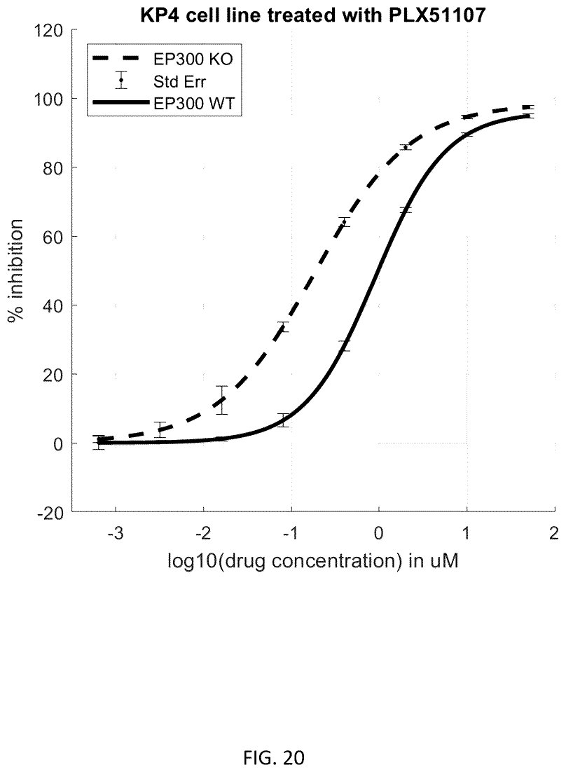

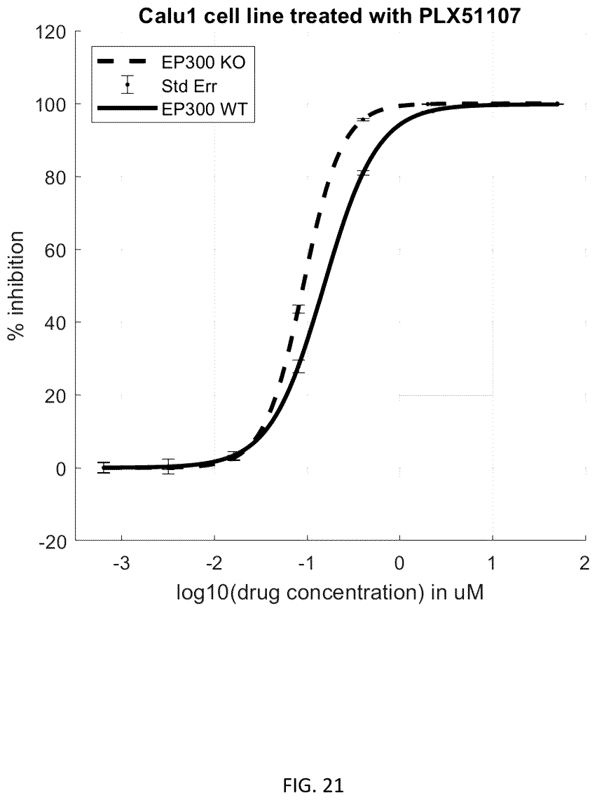

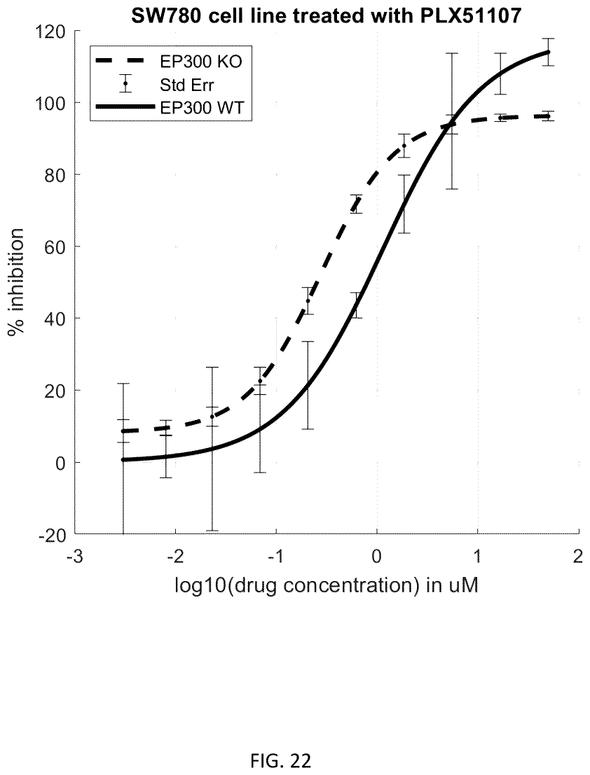

FIGS. 20-22 depict the effect of PLX-51107 in cancer cells with wild type EP300 and EP300 knocked out. FIG. 20 depicts the effect of PLX-51107 in KP4 pancreatic cancer cells with wild type EP300 and EP300 knocked out. FIG. 21 depicts the effect of PLX-51107 in Calu1 lung cancer cells with wild type EP300 and EP300 knocked out. FIG. 22 depicts the effect of PLX-51107 in SW780 bladder cancer cells with wild type EP300 and EP300 knocked out.

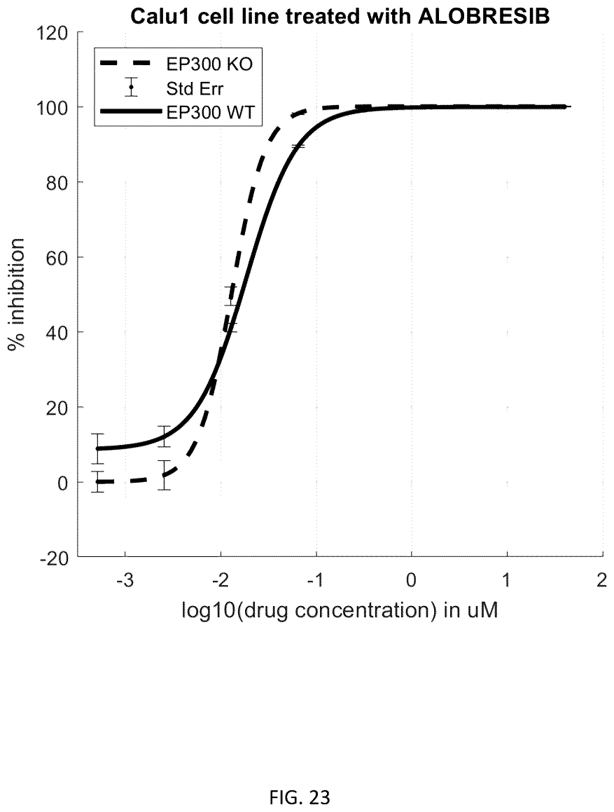

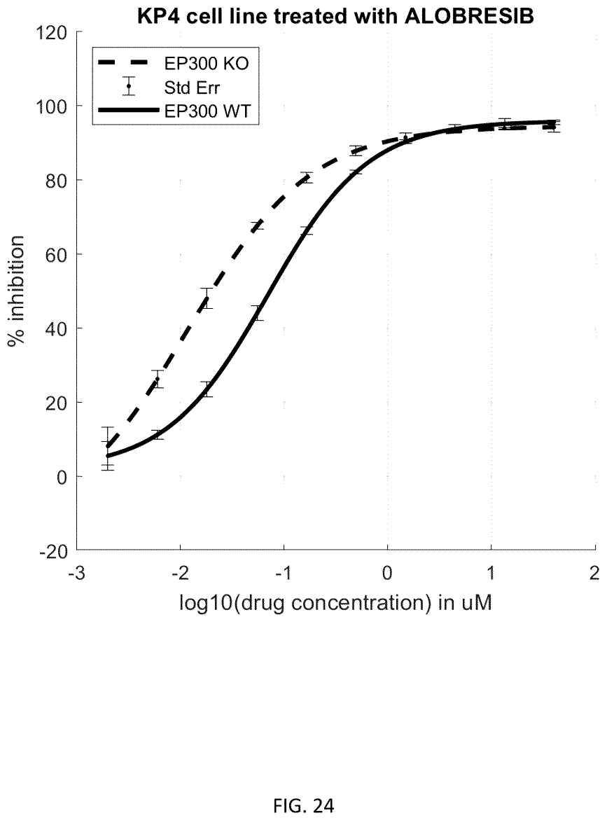

FIGS. 23-24 depict the effect of Alobresib in cancer cells with wild type EP300 and EP300 knocked out. FIG. 23 depicts the effect of Alobresib in Calu1 lung cancer cells with wild type EP300 and EP300 knocked out. FIG. 24 depicts the effect of Alobresib in KP4 pancreatic cancer cells with wild type EP300 and EP300 knocked out.

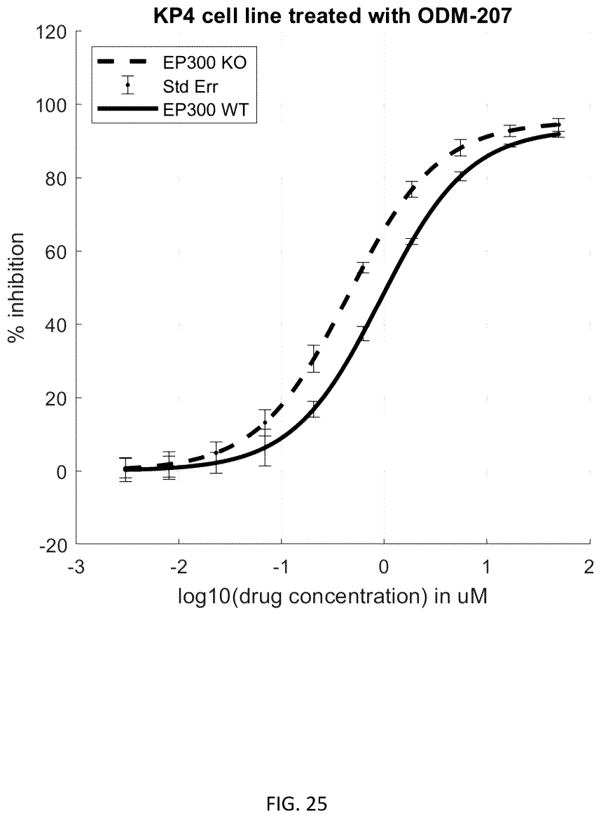

FIG. 25 depicts the effect of ODM-207 in KP4 pancreatic cancer cells with wild type EP300 and EP300 knocked out.

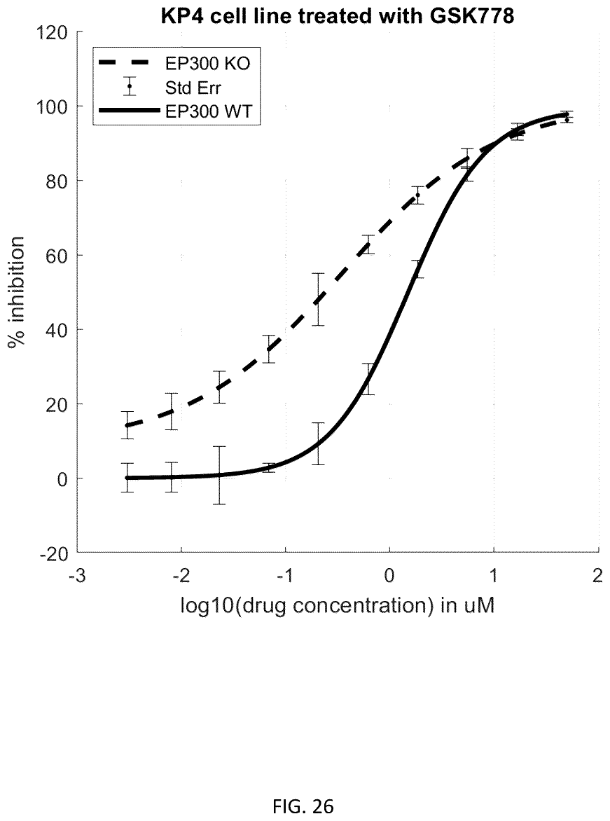

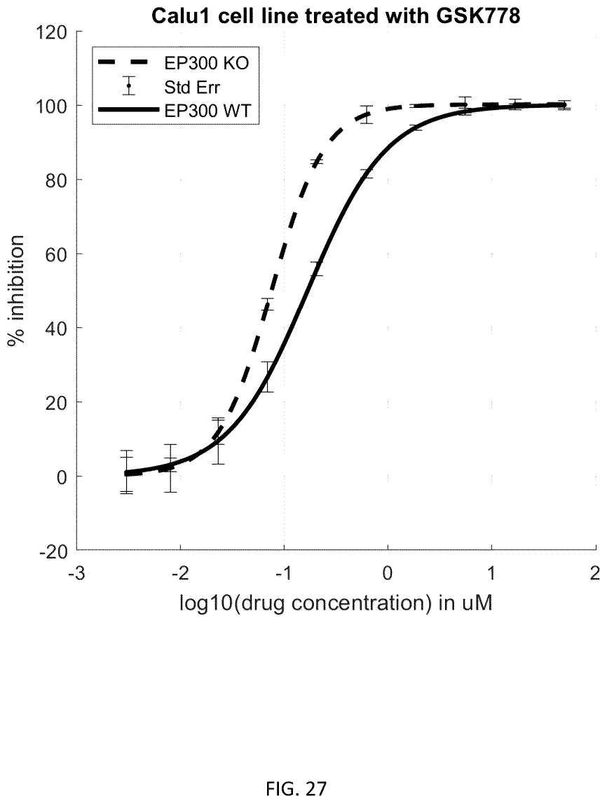

FIGS. 26-27 depict the effect of GSK778 in cancer cells with wild type EP300 and EP300 knocked out. FIG. 26 depicts the effect of GSK778 in KP4 pancreatic cancer cells with wild type EP300 and EP300 knocked out. FIG. 27 depicts the effect of GSK778 in Calu1 lung cancer cells with wild type EP300 and EP300 knocked out.

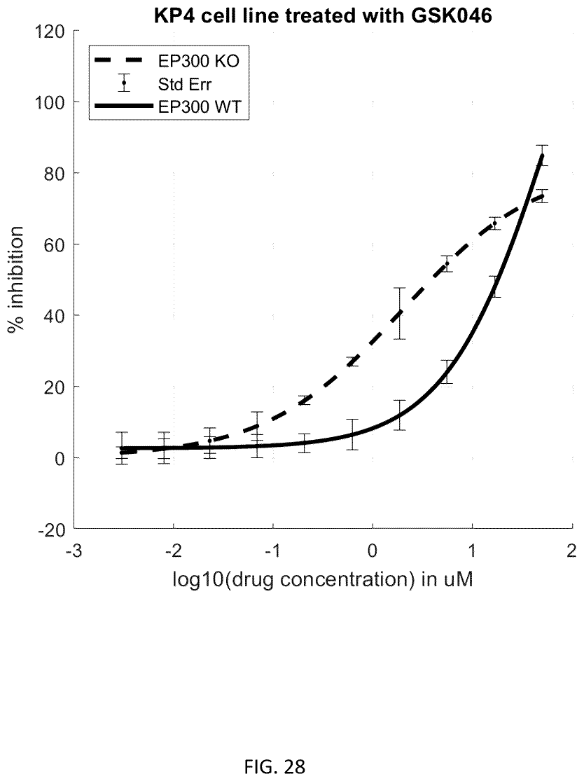

FIG. 28 depicts the effect of GSK046 in KP4 pancreatic cancer cells with wild type EP300 and EP300 knocked out.

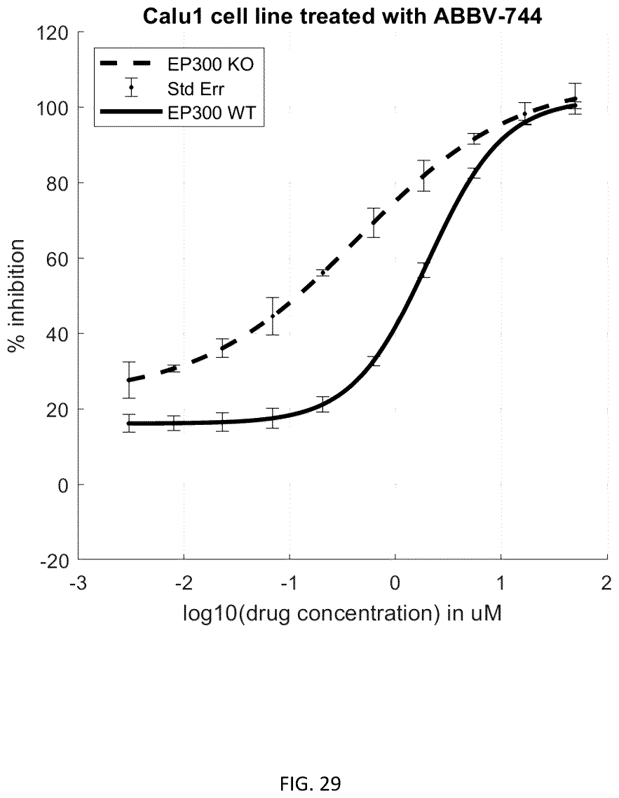

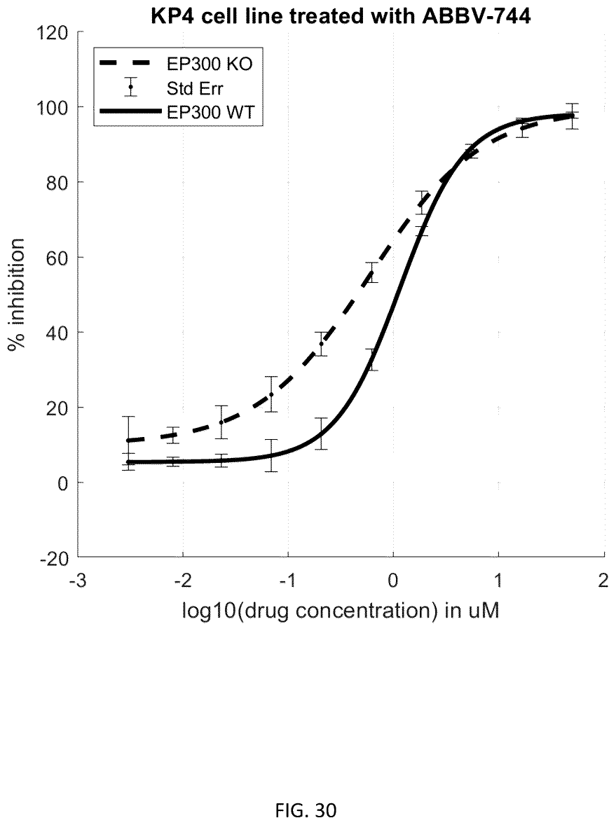

FIGS. 29-30 depict the effect of ABBV-744 in cancer cells with wild type EP300 and EP300 knocked out. FIG. 29 depicts the effect of ABBV-744 in Calu1 lung cancer cells with wild type EP300 and EP300 knocked out. FIG. 30 depicts the effect of ABBV-744 in KP4 pancreatic cancer cells with wild type EP300 and EP300 knocked out.

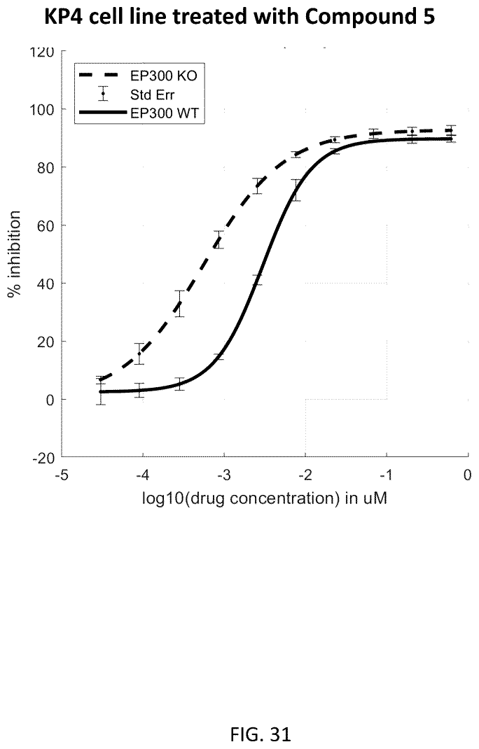

FIG. 31 depicts the effect of Compound 5 in KP4 pancreatic cancer cells with wild type EP300 and EP300 knocked out.

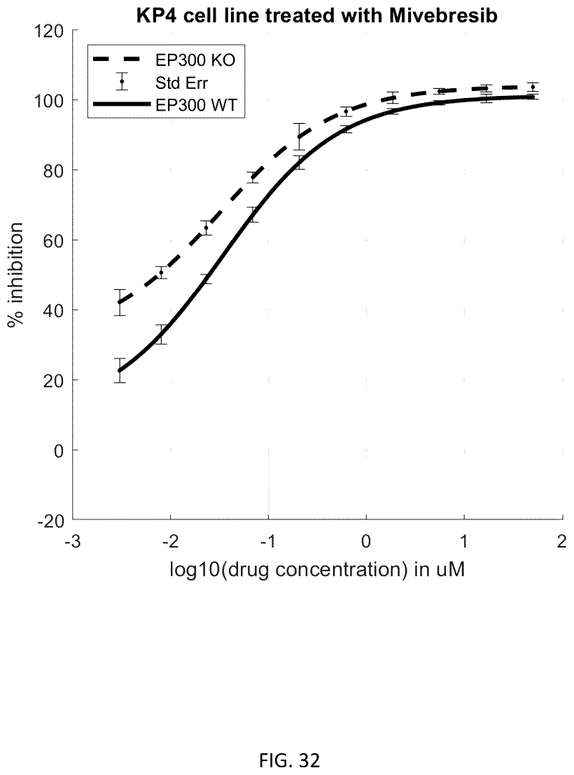

FIG. 32 depicts the effect of Mivebresib in KP4 pancreatic cancer cells with wild type EP300 and EP300 knocked out.

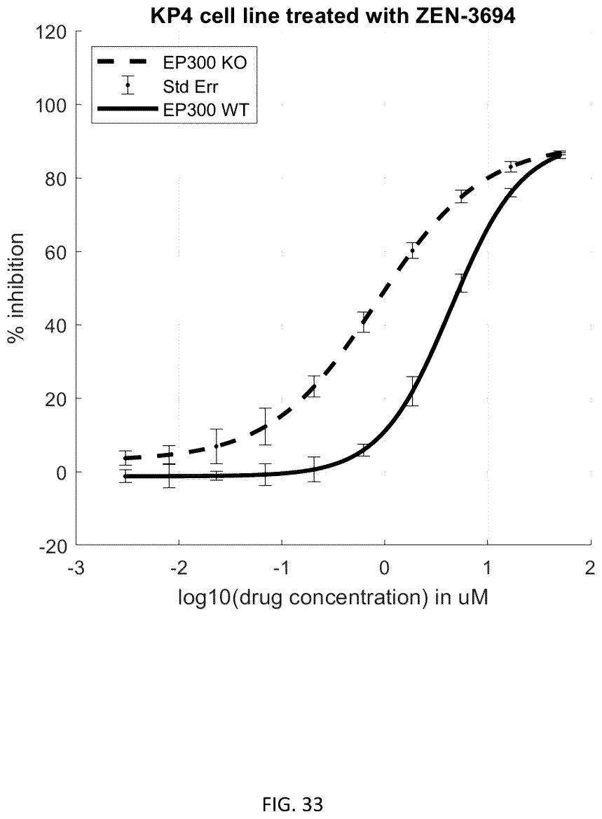

FIG. 33 depicts the effect of ZEN-3694 in KP4 pancreatic cancer cells with wild type EP300 and EP300 knocked out.

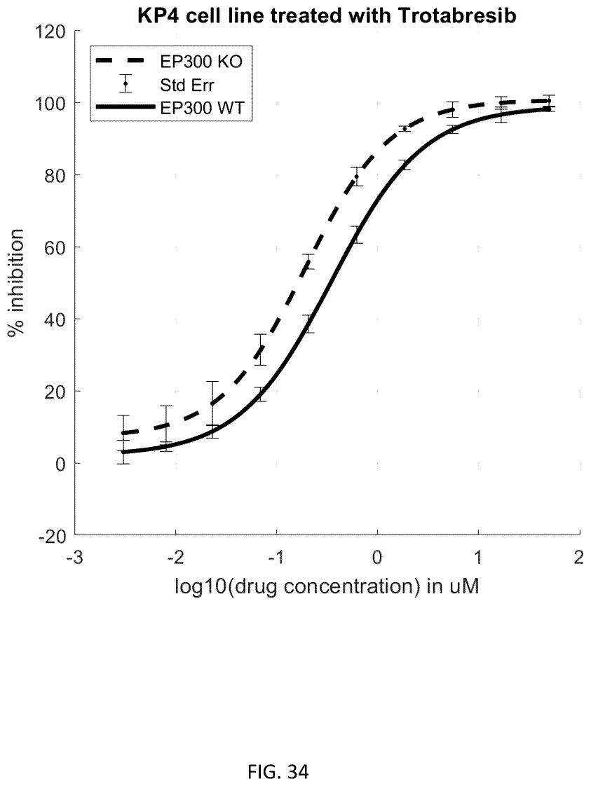

FIG. 34 depicts the effect of Trotabresib in KP4 pancreatic cancer cells with wild type EP300 and EP300 knocked out.

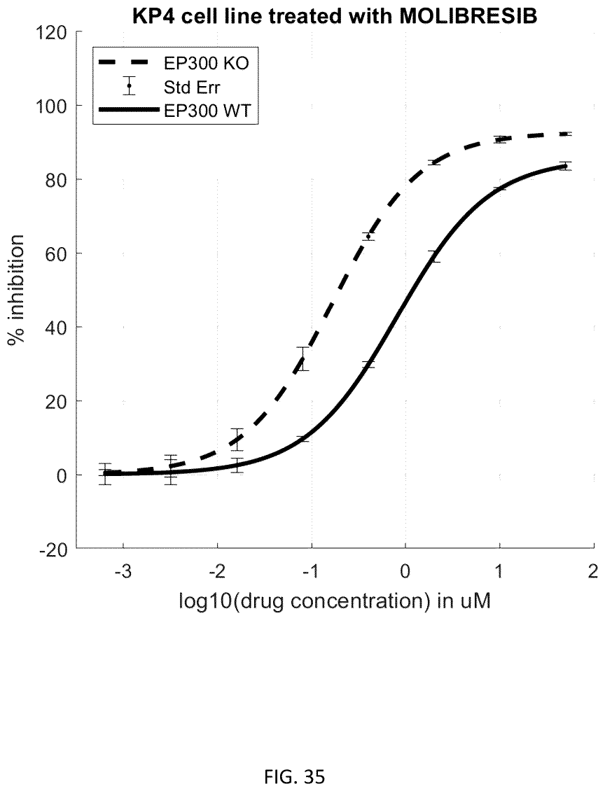

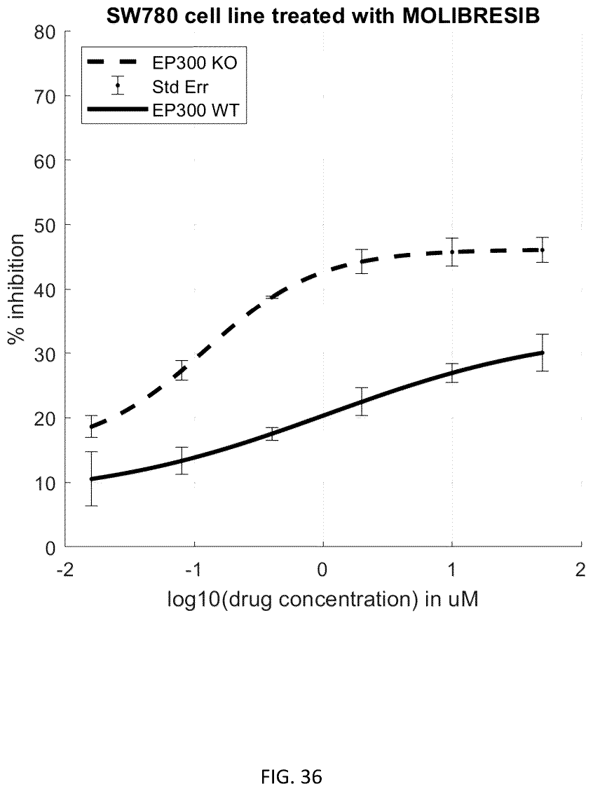

FIGS. 35-36 depict the effect of Molibresib in cancer cells with wild type EP300 and EP300 knocked out. FIG. 35 depicts the effect of Molibresib in KP4 pancreatic cancer cells with wild type EP300 and EP300 knocked out. FIG. 36 depicts the effect of Molibresib in SW780 bladder cancer cells with wild type EP300 and EP300 knocked out.

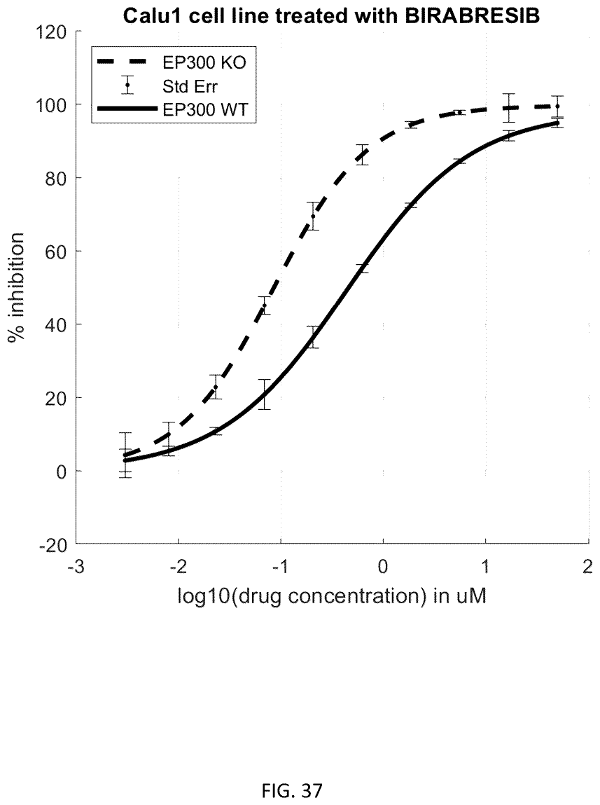

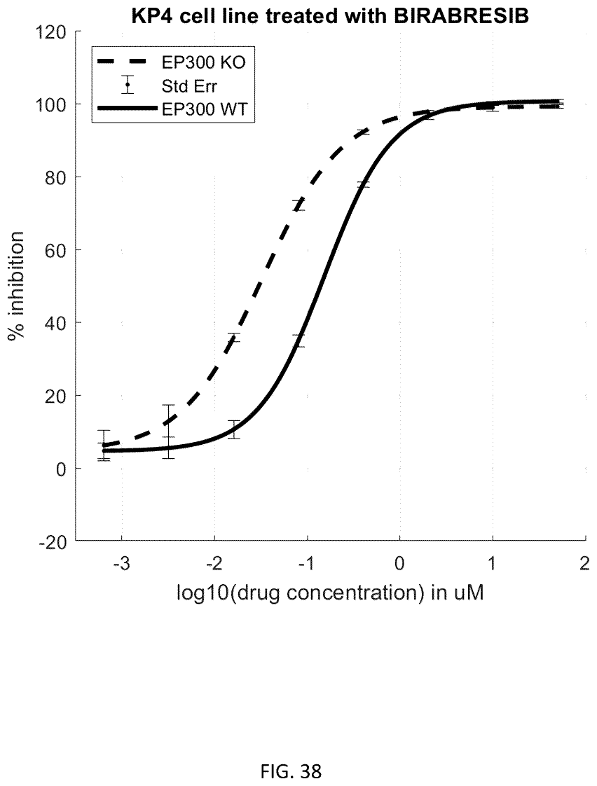

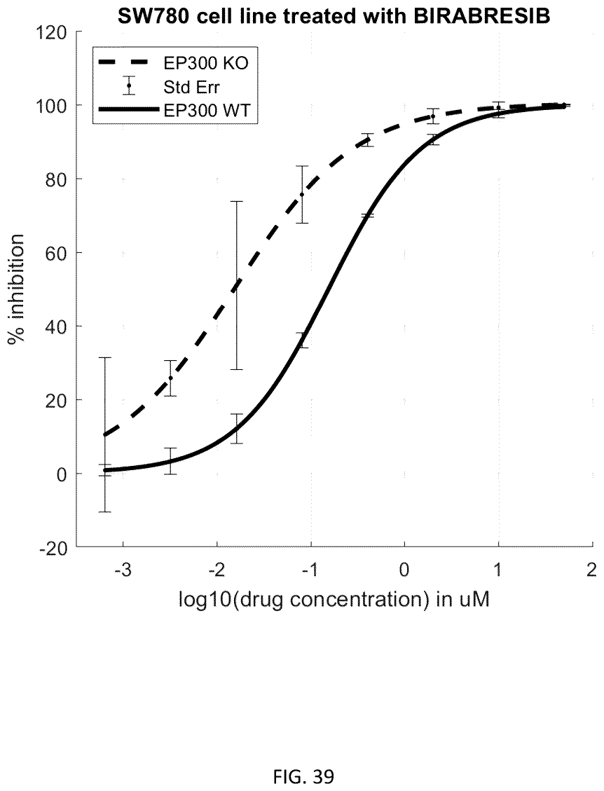

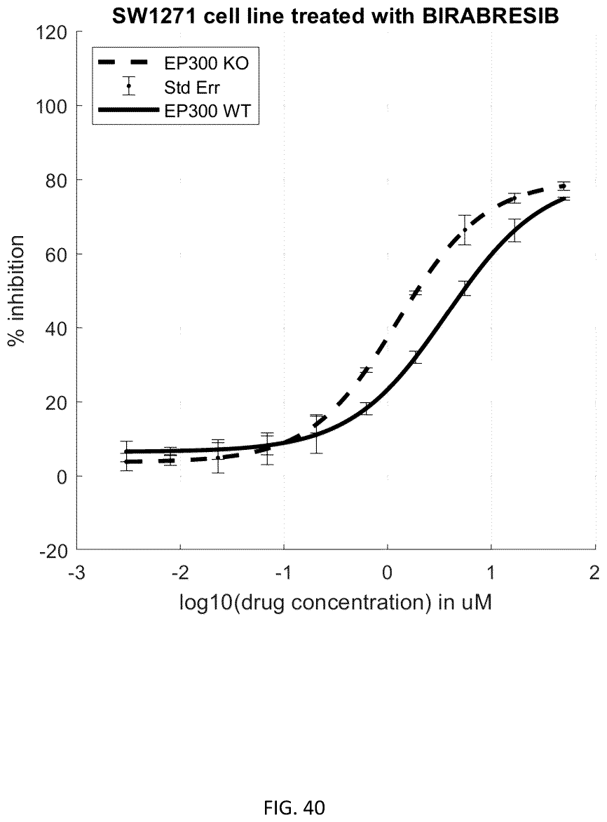

FIGS. 37-40 depict the effect of Birabresib in cancer cells with wild type EP300 and EP300 knocked out. FIG. 37 depicts the effect of Birabresib in Calu1 lung cancer cells with wild type EP300 and EP300 knocked out. FIG. 38 depicts the effect of Birabresib in KP4 pancreatic cancer cells with wild type EP300 and EP300 knocked out. FIG. 39 depicts the effect of Birabresib in SW780 bladder cancer cells with wild type EP300 and EP300 knocked out. FIG. 40 depicts the effect of Birabresib in SW1271 lung cancer cells with wild type EP300 and EP300 knocked out.

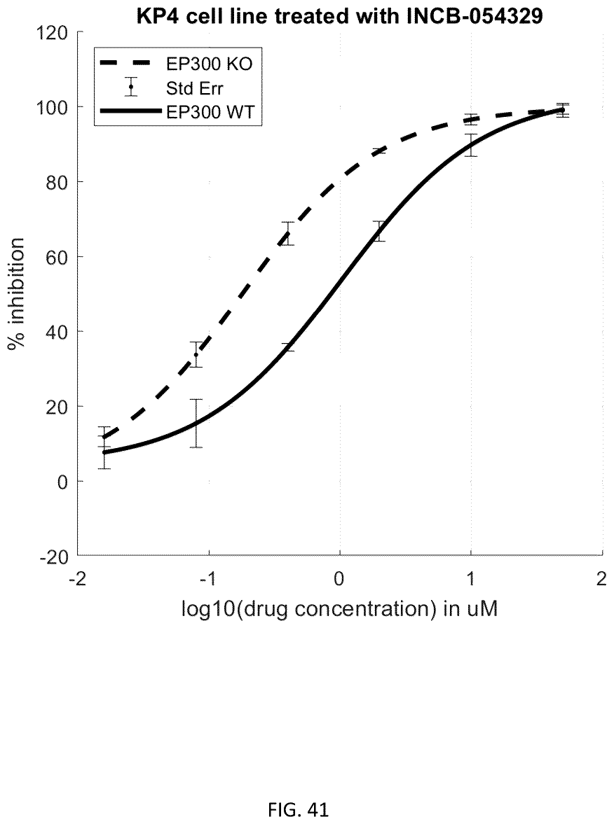

FIG. 41 depicts the effect of INCB054329 in KP4 pancreatic cancer cells with wild type EP300 and EP300 knocked out.

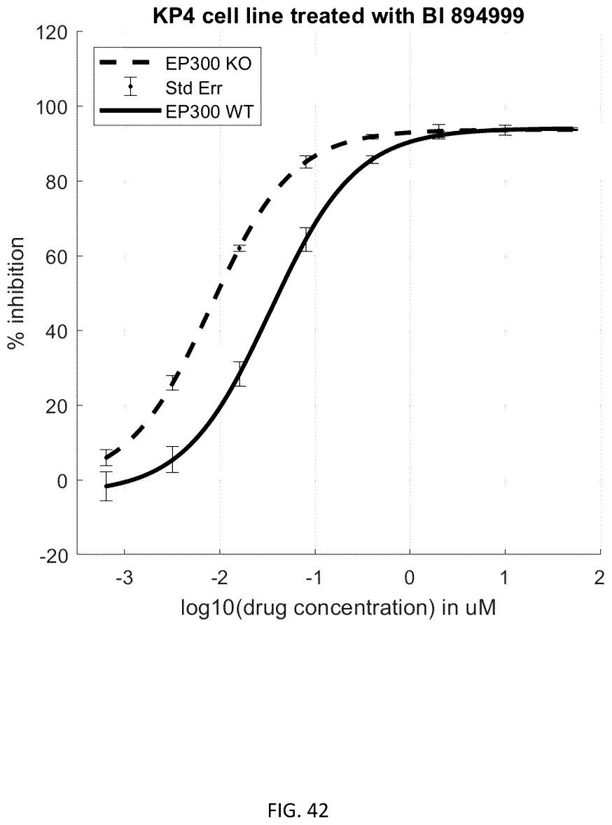

FIG. 42 depicts the effect of BI-894999 in KP4 pancreatic cancer cells with wild type EP300 and EP300 knocked out.

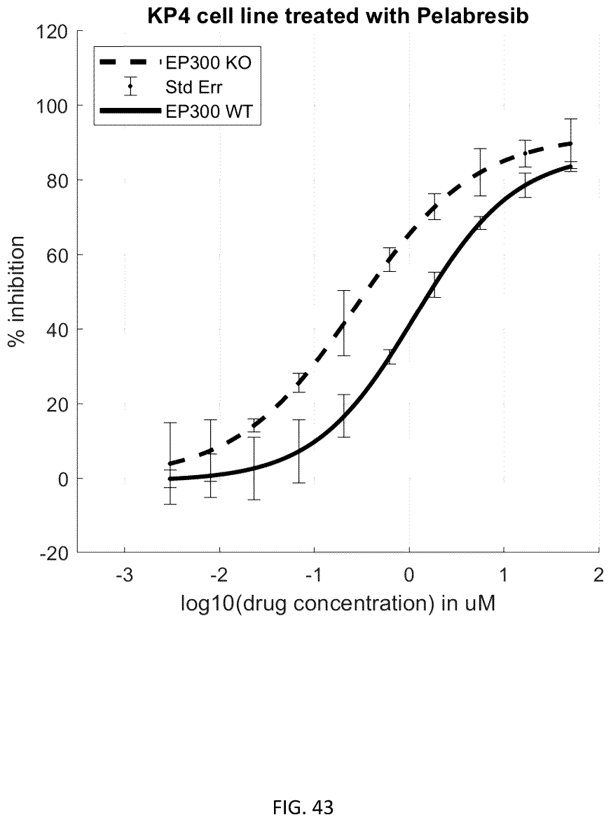

FIG. 43 depicts the effect of Pelabresib in KP4 pancreatic cancer cells with wild type EP300 and EP300 knocked out.

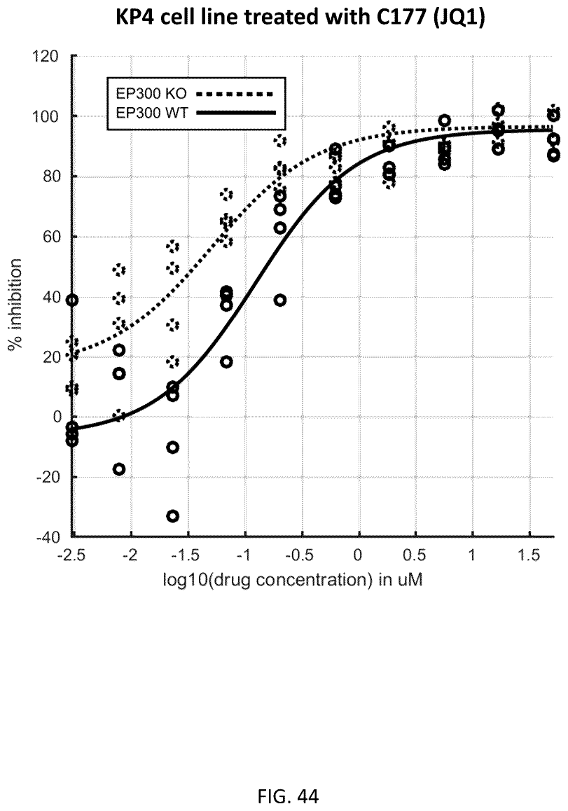

FIG. 44 depicts the effect of C177 (JQ1) in KP4 cells pancreatic cancer cells with wild type EP300 and EP300 knocked out.

The results of this experiment demonstrate that there is synthetic lethality between BET inhibitors and an EP300 loss of function mutation for drugs whose primary targets include BRD2, BRD3, and/or BRD4.

### Example 8. Xenograft Study Using Patient Derived Xenograft Models

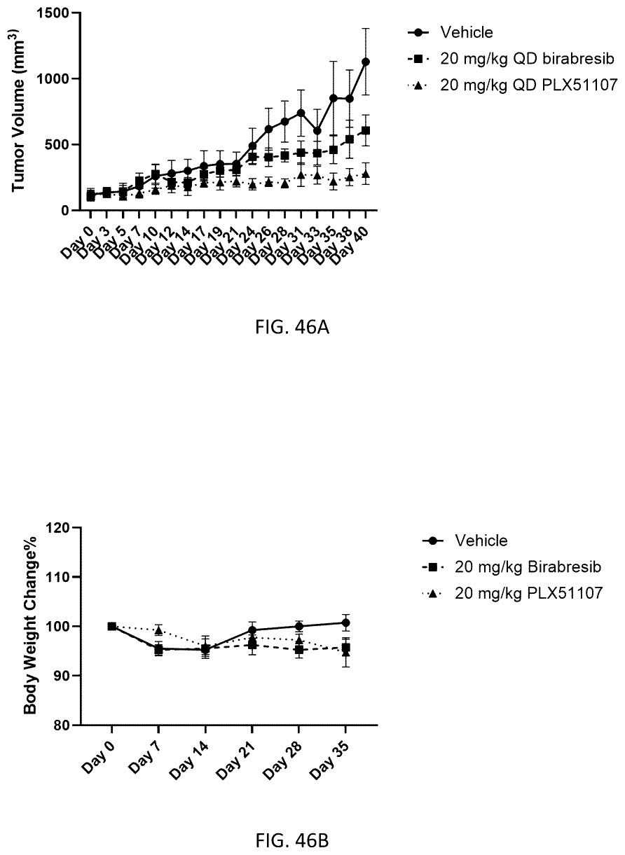

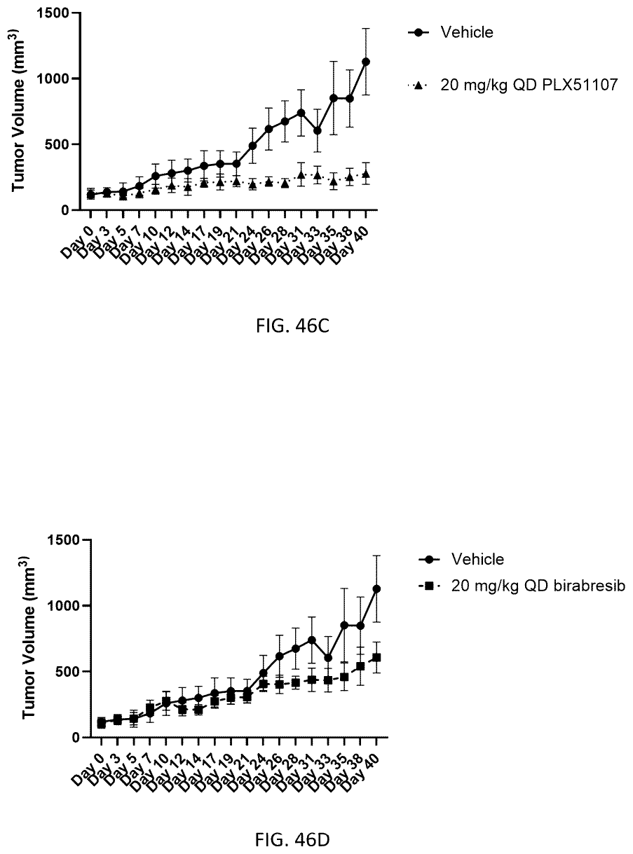

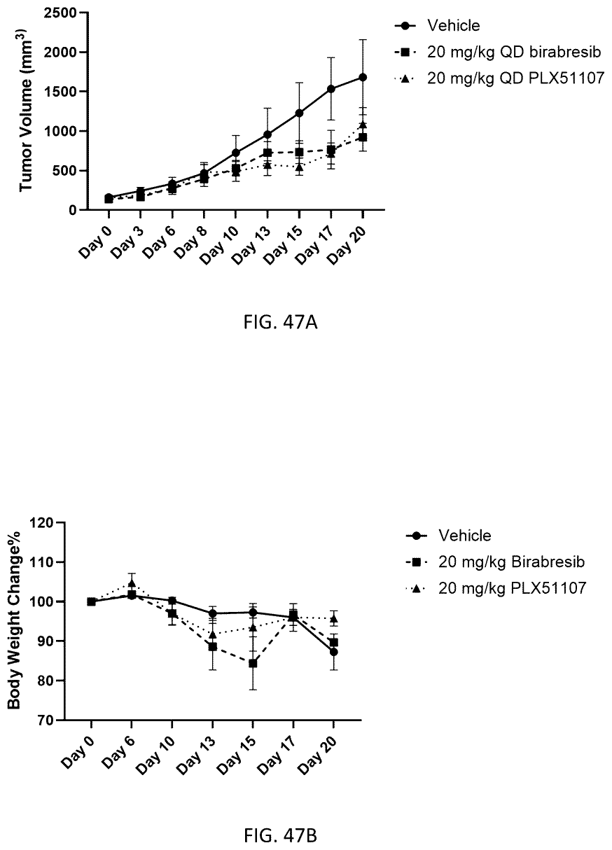

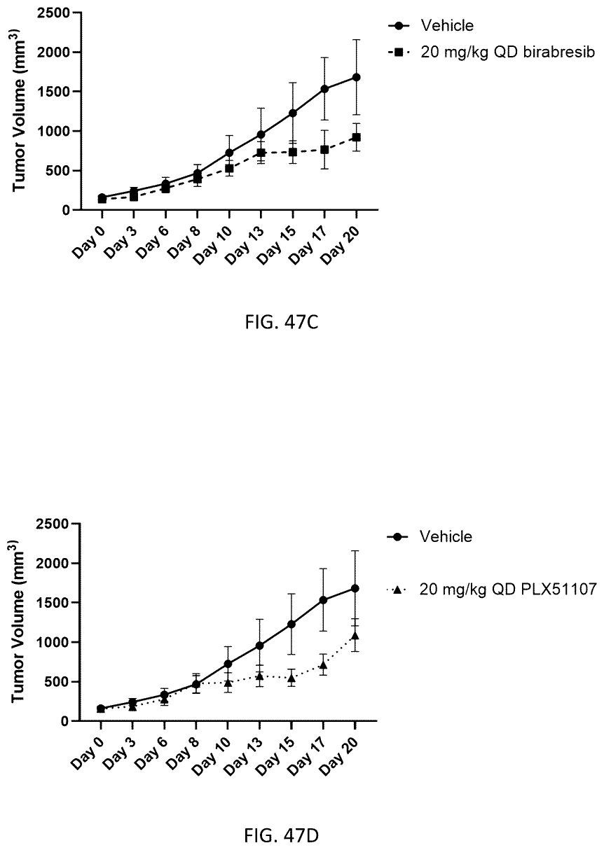

To investigate the potential for using BET inhibitors (e.g., PLX-51107 and Birabresib) to treat tumors with EP300-LOF, patient derived xenograft (PDX) models with predicted LOF mutations in EP300 were identified from the NCI Patient Derived Model Repository (PDMR) and at Jackson Labs (JAX). For these experiments, PDX models J000108112 (FIG. 46A-D) and TM00244 (FIG. 47A-D) were tested. Analysis of whole exome sequencing data to identify LOF EP300 mutations were done using methods, such as those described in Example 1. GATK and Samtools were used to call likely somatic single nucleotide variants (SNVs) and indels, which were then filtered on high likelihood of inducing a protein functional change, and further distinguished from gain of function (GOF) mutations on not being in sequence or structural hotspot regions.

PDX samples harboring a EP300 LOF mutation, selected using the criteria described above, were cut to a uniform size and implanted subcutaneously into bilateral flanks of 5-week old NSG mice sourced from JAX. For each study (i.e. each model), a 3×8 (3 treatments, 8 replicates) experimental design was employed. Tumors were measured every other day until tumor volume reached approximately 150 mm³. BET inhibitor was administered (20 mg/kg) once daily by oral gavage. Tumor sizes were recorded daily, and body weights were measured every 7 days to monitor for drug toxicity. Tumor volume was calculated using the following formula: tumor volume (mm3)=½ (W)2×(L). Mice were sacrificed once tumors reached 500 mm³. Differential outcomes were determined through a log rank Kaplan-Meier survival analysis or by comparing growth kinetics over time.

Mice harboring an EP300-LOF mutant PDX model J000108112 treated orally with either PLX-51107 or Birabresib achieved significantly reduced tumor growth (FIG. 46A). No significant body weight change is observed in mice treated with PLX-51107 or Birabresib (FIG. 46B). The treatments PLX-51107 and Birabresib were individually graphed in FIG. 46C and FIG. 46D. The results demonstrated the BET inhibitors reduced growth in the EP300-LOF mutant PDX model J000108112 mice.

Mice harboring an EP300-LOF mutant PDX model TM00244 treated orally with either PLX-51107 or Birabresib achieved significantly reduced tumor growth (FIG. 47A). No significant body weight change is observed in mice treated with PLX-51107 or Birabresib (FIG. 47B). The treatments PLX-51107 and Birabresib were individually graphed in FIG. 47C and FIG. 47D. The results demonstrated the BET inhibitors reduced growth in the EP300-LOF mutant PDX model TM00244 mice.

### Example 9. CRISPR Pharmacogenetic Screen

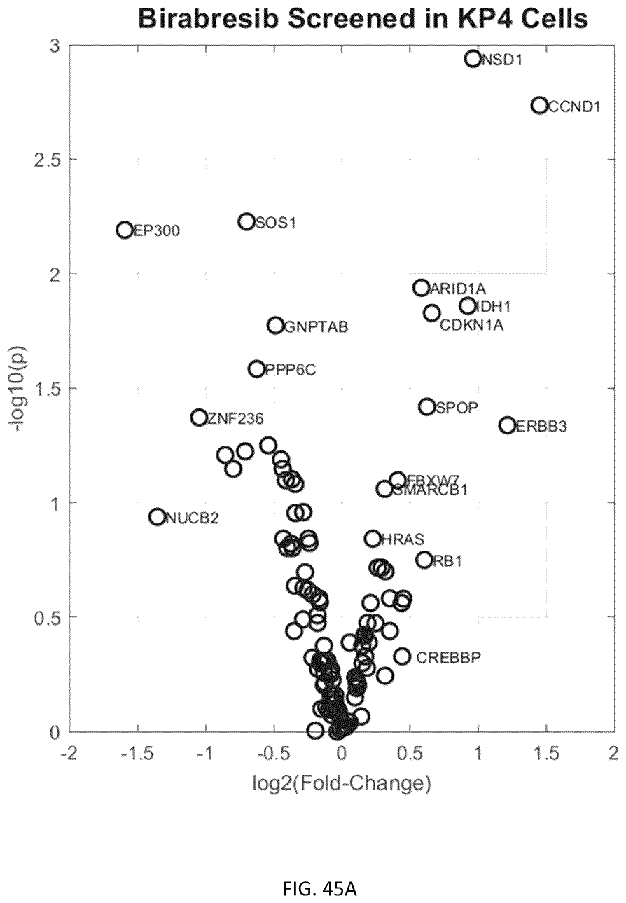

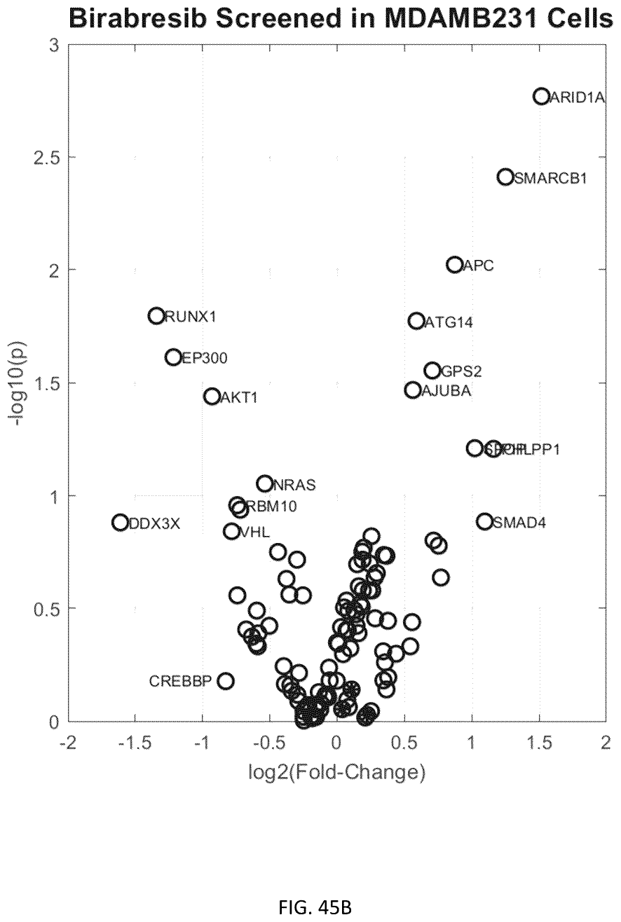

To study the effect of synthetic lethality, a CRISPR pharmacogenetic screen was performed to investigate the change in fitness of cells when a given gene is knocked out and treated with a BET inhibitor, e.g. Birabresib. Each gene knock out was accomplished via 3 to 4 different guide RNAs in KP4 cells (FIG. 45A) and MDAMB231 cells (FIG. 45B). Volcano plots were generated depicting the effect size and significance of driver gene perturbations the pooled CRISPR Birabresib pharmacogenetic screen. Each circle (point) on the plot depicts a loss of function mutation (knock out) of a specific driver gene. Points to the left of zero depict an interaction where the presence of Birabresib, but not vehicle control, lead to reduced fitness of cells having a gene perturbation. The y-axis depicts the significance of the interaction which factors in the effect size as well as the concordance among genetic perturbations.

EP300, near top left of both FIG. 45A and FIG. 45B, showed a strong and significant sensitization to Birabresib. The results further demonstrate how synthetic lethality exists between cells with an EP300 loss of function mutation or deletion and a BET inhibitor.

### Example 10. Cellular Assay Using Compounds of Formula VII (e.g., Compound 9)

To study the effect of compounds of Compound 9 on cells with a EP300 LOF mutation, cells with and without EP300 LOF mutations are treated with a compound of Formula (VII) (e.g., Compound 9) using the techniques presented in Example 3. The results of this experiment will demonstrate that there is synthetic lethality between a compound of Formula (VII) (e.g., Compound 9) and an EP300 loss of function mutation or deletion.

The examples and embodiments described herein are for illustrative purposes only and various modifications or changes suggested to persons skilled in the art are to be included within the spirit and purview of this application and scope of the appended claims.

We claim:

1. A method of treating a cancer in a subject in need thereof, said method comprising administering a pharmaceutical composition to said subject,
a. wherein said pharmaceutical composition comprises a bromodomain and extra-terminal domain (BET) inhibitor;
b. said cancer has previously been determined to comprise a loss of function or a deletion of an EP300 gene;
c. said patient is not concurrently receiving 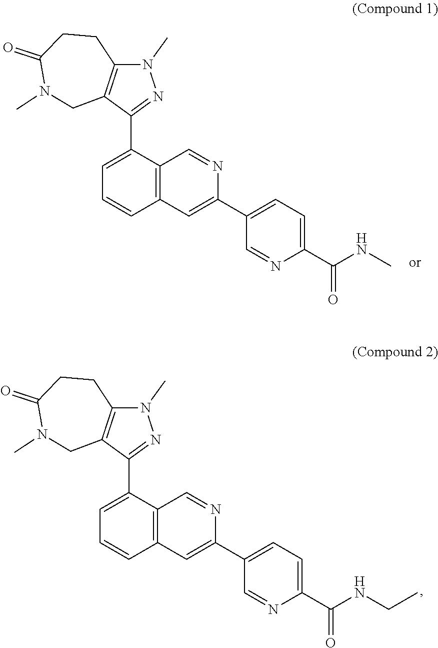 [SMILES: CNC(=O)c1ccc(-c2cc3cccc(-c4nn(C)c5c4CN(C)C(=O)CC5)c3cn2)cn1 ; CCNC(=O)c1ccc(-c2cc3cccc(-c4nn(C)c5c4CN(C)C(=O)CC5)c3cn2)cn1]
and
d. said BET inhibitor is not JQ1 when said cancer is triple negative breast cancer.

2. A method of treating a cancer in a subject in need thereof, said method comprising administering a pharmaceutical composition to said subject,
wherein said pharmaceutical composition comprises a bromodomain and extra-terminal domain (BET) inhibitor;
said cancer has previously been determined to comprise a decreased amount or activity of p300 compared to wild type p300;
said patient is not concurrently receiving 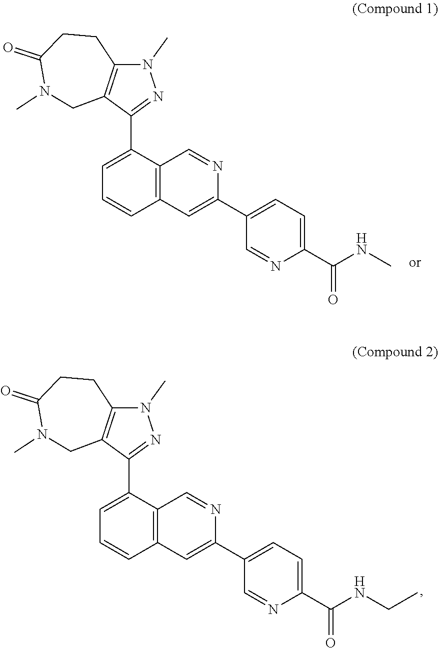 [SMILES: CNC(=O)c1ccc(-c2cc3cccc(-c4nn(C)c5c4CN(C)C(=O)CC5)c3cn2)cn1 ; CCNC(=O)c1ccc(-c2cc3cccc(-c4nn(C)c5c4CN(C)C(=O)CC5)c3cn2)cn1]
and
said BET inhibitor is not JQ1 when said cancer is triple negative breast cancer.

3. The method of claim 1, wherein said loss of function is caused by genetic mutation.

4. The method of claim 1 or 2, wherein said subject has not previously received Compound 1 or Compound 2.

5. The method of any one of claims 1-4, wherein said BET inhibitor comprises ABBV-075, ABBV-744, Apabetalone, APL-581, ARV-825, AZD-5153, BI-6727, BI-894999, BMS-986158, BOS-475, BPI-23314, CD-161, CG-223, CK-103, CN-470, FT-1101, GNE-0011, GS-5829, GS-626510, GSK525762, I-BET151, INCB054329, INCB57643, JQ1, LY-294002, NEO2734, ODM-207, OMT-001, OMT-002, OTX-015, Pelabresib, PLX-2853 (OPN-2853), PLX-51107 (OPN-51107), TEN-010, RVX-297, SRX-2523, SRX-3225, SRX-3254, SW-064652, SYHA-1801, TTI-281, Zen-3694, CPI-0610, INCB0543294, INCB057643, CC-90010, Alobresib, GSK778, GSK046, Mivebresib, Trotabresib, Molibresib, Pelabresib or a combination thereof.

6. The method of claim 5, wherein said BET inhibitor comprises AZD-5153, BI-894999, BMS-986158, GSK525762, INCB054329, INCB57643, OTX-015, PLX-51107, or a combination thereof.

7. The method of claim 5, wherein said BET inhibitor comprises AZD-5153, BI-894999, BMS-986158, GSK525762, INCB054329, OTX-015, PLX-51107, INCB057643, TEN-010, CC-90010, Alobresib, ODM-207, GSK778, GSK046, ABBV-744, Mivebresib, Trotabresib, Molibresib, Birabresib, Pelabresib, or a combination thereof.

8. The method of any one of claims 1-4, wherein said BET inhibitor comprises a compound of Formula (I): 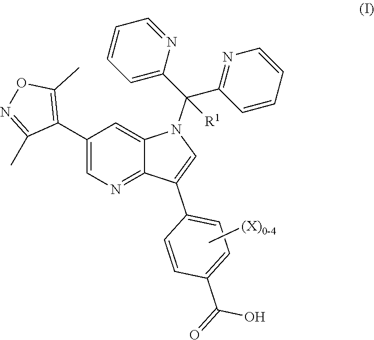
or a pharmaceutically acceptable salt, a tautomer, a stereoisomer or a deuterated analog thereof, wherein:
R¹ is cyano, halo, or (C₁-C₃)alkyl optionally substituted with 1 to 3 substituents independently selected from the group consisting of halo, methyl, ethyl, methoxy and ethoxy; and
X, when present, is halo.

9. The method of claim 7, wherein R¹ is (C₁-C₂)alkyl, cyano or fluoro.

10. The method of claim 9, wherein R¹ is methyl.

11. The method of claim 9, wherein R¹ is fluoro.

12. The method of claim 9, wherein R¹ is cyano.

13. The method of any one of claims 1-4, wherein said BET inhibitor comprises a compound of Formula (II): 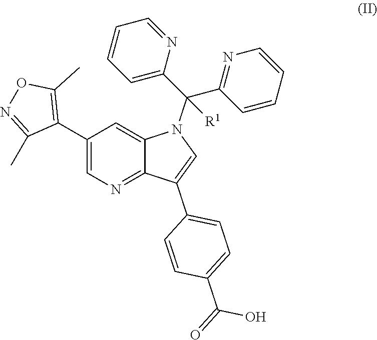
or a pharmaceutically acceptable salt, a tautomer, a stereoisomer, or a deuterated analog thereof, wherein:
R¹ is (C₁-C₃)alkyl optionally substituted with 1 to 3 substituents independently selected from the group consisting of halo, methyl, ethyl, methoxy and ethoxy.

14. The method of claim 13, wherein R¹ is methyl.

15. The method of any one of claims 1-4, wherein said BET inhibitor comprises a compound of Formula (III): 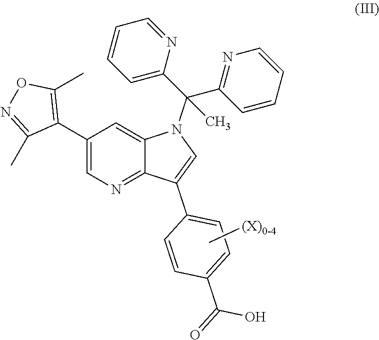
or a pharmaceutically acceptable salt thereof, wherein:
X, when present, is halo.

16. The method of any one of claims 1-4 and 7-15, wherein said BET inhibitor comprises Compound 3: 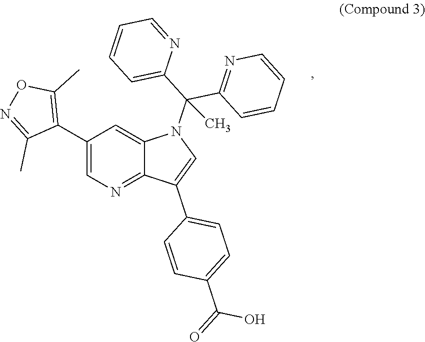 [SMILES: Cc1noc(C)c1-c1cnc2c(-c3ccc(C(=O)O)cc3)cn(C(C)(c3ccccn3)c3ccccn3)c2c1]
or a pharmaceutically acceptable salt, a tautomer, a solvate, or a deuterated analog thereof.

17. The method of any one of claims 1-4, wherein said BET inhibitor comprises a compound having formula (Va): 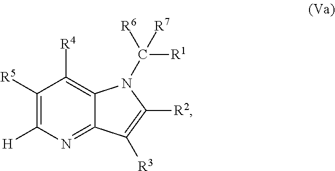
or a pharmaceutically acceptable salt, a solvate, a tautomer, a stereoisomer or a deuterated analog thereof,
wherein:
R₂ is H;
R₄ is H;
R₆ is H;
R⁷ is H, OH, C₁₋₆ alkyl, aryl, heteroaryl, cycloalkyl, or heterocycloalkyl;
R¹ is heteroaryl optionally substituted with from 1-3 R^{j} groups;
each R^{j} independently selected from halogen, —CN, —OH, —NH₂, —NO₂, —C(O)OH, —C(S)OH, —C(O)NH₂, —C(S)NH₂, —S(O)₂NH₂, —NHC(O)NH₂, —NHC(S)NH₂, —NHS(O)₂NH₂, —C(NH)NH₂, —CH═C(R^{k})(R^{k}), —OR^{k}, —SR^{k}, —OC(O)R^{k}, —OC(S)R^{k}, —P(═O)HR^{k}, —P(═O)R^{k}R^{k}, —PH(═O)OR^{k}, —P(═O)(OR^{k})₂, —OP(═O)(OR^{k})₂, —C(O)H, —O(CO)OR^{k}, —C(O)R^{k}, —C(S)R^{k}, —C(O)OR^{k}, —C(S)OR^{k}, —S(O)R^{k}, —S(O)₂R^{k}, —C(O)NHR^{k}, —C(S)NHR^{k}, —C(O)NR^{k}R^{k}, —C(S)NR^{k}R^{k}, —S(O)₂NHR^{k}, —S(O)₂NR^{k}R^{k}, —C(NH)NHR^{k}, —C(NH)NR^{k}R^{k}, —NHC(O)R^{k}, —NHC(S)R^{k}, —NR^{k}C(O)R^{k}, —NR^{k}C(S)R^{k}, —NHS(O)₂R^{k}, —NR^{k}S(O)₂R^{k}, —NHC(O)NHR^{k}, —NHC(S)NHR^{k}, —NR^{k}C(O)NH₂, —NR^{k}C(S)NH₂, —NR^{k}C(O)NHR^{k}, —NR^{k}C(S)NHR^{k}, —NHC(O)NR^{k}R^{k}, —NHC(S)NR^{k}R^{k}, —NR^{k}C(O)NR^{k}R^{k}, —NR^{k}C(S)NR^{k}R^{k}, —NHS(O)₂NHR^{k}, —NR^{k}S(O)₂NH₂, —NR^{k}S(O)₂NHR^{k}, —NHS(O)₂NR^{k}R^{k}, —NR^{k}S(O)₂NR^{k}R^{k}, —NHR^{k} or —NR^{k}R^{k};
each R^{k} is independently H, C₁₋₆alkyl, aryl, arylalkyl, heteroaryl, heteroarylalkyl, heterocycloalkyl, heterocycloalkylalkyl, cycloalkyl or cycloalkylalkyl; or
two R^{k} groups when attached to the same carbon or nitrogen atom are taken together to form a 3- to 6-membered carbocyclic ring or 3- to 8-membered heterocyclic ring having from 1-2 heteroatoms as ring members selected from O, N or S, wherein the nitrogen or sulfur ring atoms are optionally oxidized;
R³ is H, halogen, —CN, optionally substituted C₁₋₆alkyl, optionally substituted deuterated C₁₋₆alkyl, optionally substituted aryl, optionally substituted aryl-C₁₋₄alkyl, optionally substituted heteroaryl, optionally substituted heteroaryl-C₁₋₄alkyl, optionally substituted C₃₋₈ cycloalkyl, optionally substituted C₃₋₈ cycloalkyl-C₁₋₄alkyl, optionally substituted heterocycloalkyl, or optionally substituted heterocycloalkyl-C₁₋₄alkyl; and
R⁵ is 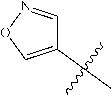 [SMILES: *c1cnoc1]
optionally substituted with from 1 to 2 R¹¹ groups independently selected from D, halogen, C₁₋₆alkyl, C₁₋₄haloalkyl, C₁₋₄haloalkoxy or —CN;
wherein the wavy line indicates the point of attachment to the rest of molecule.

18. The method of claim 17, wherein said BET inhibitor comprises Compound 4: 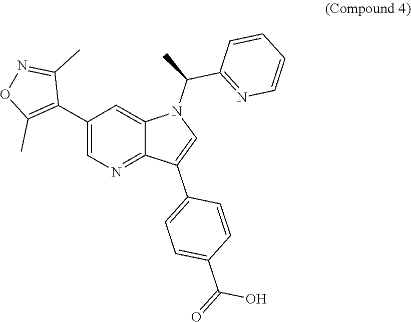 [SMILES: Cc1noc(C)c1-c1cnc2c(-c3ccc(C(=O)O)cc3)cn([C@@H](C)c3ccccn3)c2c1]
or a pharmaceutically acceptable salt, a tautomer, a solvate, or a deuterated analog thereof.

19. The method of any one of claims 1-4, wherein said BET inhibitor comprises Compound 9: 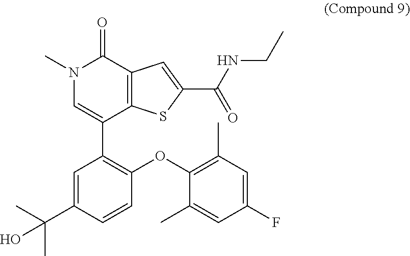 [SMILES: CCNC(=O)c1cc2c(=O)n(C)cc(-c3cc(C(C)(C)O)ccc3Oc3c(C)cc(F)cc3C)c2s1]
or a pharmaceutically acceptable salt, a tautomer, a solvate, or a deuterated analog thereof.

20. The method of any one of the previous claims, wherein said cancer comprises bladder cancer, lung cancer, gynecologic cancer, adrenocortical carcinoma, bone cancer, central nervous system (CNS) cancer, pancreatic cancer, gastrointestinal cancer, head and neck cancer, skin cancer, mesothelioma, nerve sheath tumor, lymphoma, renal cell carcinoma, salivary gland cancer, non-melanoma skin cancer, or a combination thereof.

21. The method of any one of claims 1-20, wherein said cancer comprises lung cancer.

22. The method of claim 21, wherein said lung cancer comprises non-small cell lung cancer.

23. The method of claim 22, wherein said non-small cell lung cancer comprises squamous cell carcinoma, large cell carcinoma, adenocarcinoma, or a combination thereof.

24. The method of claim 23, wherein said non-small cell lung cancer comprises squamous cell carcinoma.

25. The method of claim 23, wherein said non-small cell lung cancer comprises large cell carcinoma.

26. The method of claim 23, wherein said non-small cell lung cancer comprises adenocarcinoma.

27. The method of claim 21, wherein said lung cancer comprises small cell lung cancer.

28. The method of any one of claims 21-27, wherein said BET inhibitor comprises AZD-5153, INCB57643, OTX-015, PLX-51107, or a combination thereof.

29. The method of any one of claims 1-20, wherein said cancer comprises bladder cancer.

30. The method of claim 29, wherein said bladder cancer comprises a carcinoma.

31. The method of claim 30, wherein carcinoma comprises urothelial carcinoma.

32. The method of any one of claims 29-31, wherein said BET inhibitor comprises AZD-5153, BMS-986158, GSK525762, INCB57643, OTX-015, PLX-51107, or a combination thereof.

33. The method of any one of claims 1-20, wherein said cancer comprises pancreatic cancer.

34. The method of claim 33, wherein said BET inhibitor comprises BI-894999, BMS-986158, GSK525762, INCB054329, INCB57643, OTX-015, PLX-51107, or a combination thereof.

35. The method of claim 20, wherein said skin cancer comprises melanoma.

36. The method of claim 20, wherein said CNS cancer comprises a glioma.

37. The method of claim 20, wherein said CNS cancer comprises a neuroepithelial tumor.

38. The method of claim 20, wherein said lymphoma comprises Non-Hodgkin lymphoma.

39. The method of claim 20, wherein said lymphoma comprises diffuse large B cell lymphoma.

40. The method of claim 20, wherein said lymphoma comprises follicular lymphoma.

41. The method of claim 20, wherein said lymphoma comprises marginal zone lymphoma.

42. The method of claim 20, wherein said lymphoma comprises mature B-cell neoplasm.

43. The method of claim 20, wherein said gastrointestinal cancer comprises a gastrointestinal stromal tumor, esophagogastric cancer, gastrointestinal neuroendocrine tumor, small bowel cancer, anal cancer, colon cancer, or a combination thereof.

44. The method of claim 20, wherein said gastrointestinal cancer comprises a gastrointestinal stromal tumor.

45. The method of claim 20, wherein said gastrointestinal cancer comprises an esophagogastric cancer.

46. The method of claim 20, wherein said gastrointestinal cancer comprises a gastrointestinal neuroendocrine tumor.

47. The method of claim 20, wherein said gastrointestinal cancer comprises a small bowel cancer.

48. The method of claim 20, wherein said gastrointestinal cancer comprises an anal cancer.

49. The method of claim 20, wherein said gastrointestinal cancer comprises colon cancer.

50. The method of claim 49, wherein said colon cancer comprises colorectal cancer.

51. The method of claim 20, wherein said gynecologic cancer comprises a cervical cancer.

52. The method of claim 20, wherein said gynecologic cancer comprises an ovarian cancer.

53. The method of claim 20, wherein said gynecologic cancer comprises a sex cord stromal tumor.

54. The method of claim 20, wherein said gynecologic cancer comprises a vaginal cancer.

55. The method of claim 20, wherein said cancer comprises uterine cancer.

56. The method of claim 55, wherein said uterine cancer comprises endometrial cancer or uterine sarcoma.

57. The method of claim 56, wherein said uterine cancer comprises endometrial cancer.

58. The method of claim 57, wherein said endometrial cancer comprises uterine corpus endometrial carcinoma.

59. The method of claim 55, wherein said uterine cancer comprises uterine sarcoma.

60. The method of claim 59, wherein said uterine sarcoma comprises uterine carcinosarcoma.

61. The method of any one of the previous claims, wherein said BET inhibitor inhibits any one or more of the following bromodomains: BRD2, BRD3, and BRD4.

62. The method of claim 61, wherein said BET inhibitor inhibits BRD4 and BRD2.

63. The method of claim 61, wherein said BET inhibitor inhibits BRD3 and BRD4.

64. The method of claim 61, wherein said BET inhibitor inhibits BD1 bromodomain of BRD2, BRD3, BRD4, or any combination thereof.

65. The method of claim 61, wherein said BET inhibitor inhibits BD2 bromodomain of BRD2, BRD3, BRD4, or any combination thereof.

66. The method of any one of the previous claims, wherein said BET inhibitor comprises a selective BET inhibitor.

67. The method of claim 66, wherein said selective BET inhibitor inhibits any one or more of the following bromodomains: BRD2, BRD3, and BRD4.

68. The method of claim 67, wherein said selective BET inhibitor is selective for BRD4 and BRD2.

69. The method of claim 67, wherein said selective BET inhibitor is selective for BRD2, BRD3, and BRD4.

70. The method of claim 67, wherein said selective BET inhibitor inhibits BD1 bromodomain of BRD2, BRD3, BRD4, or any combination thereof.

71. The method of claim 70, wherein said selective BET inhibitor comprises GSK778

72. The method of claim 67, wherein said selective BET inhibitor inhibits BD2 bromodomain of BRD2, BRD3, BRD4, or any combination thereof.

73. The method of claim 72, wherein said selective BET inhibitor comprises GSK046, ABBV-744, Compound 9, or any combination thereof.

74. The method of any one of the previous claims, wherein said loss of function or deletion is for an EP300 gene.

75. A method of treating a cancer in a subject in need thereof, said method comprising administering a pharmaceutical composition to said subject,
a. wherein said pharmaceutical composition comprises a bromodomain and extra-terminal domain (BET) inhibitor;
b. said cancer has previously been determined to comprise a loss of function or a deletion of an EP300 gene;
wherein said BET inhibitor comprises a compound of formula IV, 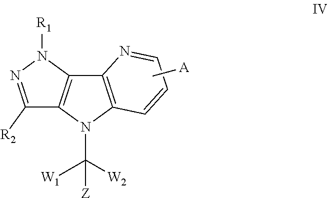
or a pharmaceutically acceptable salt thereof or a stereoisomer thereof,
wherein:
R₁ is selected from hydrogen; deuterium; —C₁₋₆alkyl; or —C₃₋₈carbocyclic; and each of which at each occurrence is independently optionally substituted with 1, 2, 3, 4, 5 or 6 substituents, and the said each of substituents at each occurrence is independently selected from deuterium, halogen, OH, —CN, —C₁₋₈alkyl, —C₁₋₈alkoxy, —NH₂, —NH(C₁₋₆ alkyl), —N(C₁₋₆alkyl)₂, or —C₃₋₈carbocyclic;
R₂ is selected from hydrogen; deuterium; halogen; —OR₂₁; —NR₂₁R₂₂; —CN; —SR₂₁; —SOR₂₁; —SO₂R₂₁; —SO₂NR₂₁R₂₂; —C₁₋₈alkyl; 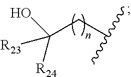
carboxyl; —COOR₂₁; —CONR₂₁R₂₂; —NR₂₁COR₂₂; —NR₂₁SO₂R₂₂; or —C₃₋₈carbocyclic; and each of which at each occurrence is independently optionally substituted with 1, 2, 3, 4, 5 or 6 substituents, and the said each of substituents at each occurrence is independently selected from deuterium, halogen, —OH, —CN, —NH₂, —C₁₋₈alkyl, —C₁₋₈alkoxy, C₃₋₈carbocyclic, or 6-membered heterocyclic containing 1, 2 or 3 heteroatoms selected from N, O;
each of R₂₁ and R₂₂ at each occurrence is independently selected from hydrogen; deuterium; —OH; NH₂; —CN; —C₁₋₈alkyl; —C₁₋₈alkoxy; —C₁₋₈alkylene-C₃₋₈carbocyclic; or —C₃₋₈carbocyclic;
each of R₂₃ and R₂₄ at each occurrence is independently selected from hydrogen, deuterium, or —C₁₋₈alkyl;
A is selected from 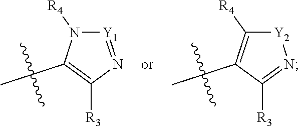
Y₁ is selected from N or CR_{Y1};
Y₂ is selected from O, S, CR_{Y1}R_{Y2} or NR_{Y2};
each of R_{Y1} and R_{Y2} at each occurrence is independently selected from hydrogen, deuterium, halogen, —OH, NH₂, —CN, —C₁₋₆alkyl or —C₁₋₆alkoxy;
each of R₃ and R₄ at each occurrence is independently selected from hydrogen, deuterium, or —C₁₋₆alkyl; and each of which at each occurrence is independently optionally substituted with 1, 2, 3, 4, 5 or 6 substituents, and the said each of substituents at each occurrence is independently selected from deuterium, halogen, —OH, —NH₂, —CN, —C₁₋₆alkyl or —C₁₋₆alkoxy;
n is selected from 0, 1, 2, 3, 4, 5 or 6;
W₁ is selected from hydrogen; deuterium; —F; —CI; —NH₂; —CN; —OH; carboxyl; —C₁₋₆ alkyl; —C₁₋₆alkoxy; —C₁₋₃alkylene-C₁₋₃alkoxy; phenyl; 5-membered heteroaryl containing 1, 2 or 3 heteroatoms selected from N, O; 6-membered heteroaryl containing 1, 2 or 3 heteroatoms selected from N, O; 3-membered heterocyclic containing 1, 2 or 3 heteroatoms selected from N, O; 4-membered heterocyclic containing 1, 2 or 3 heteroatoms selected from N, O; 5-membered heterocyclic containing 1, 2 or 3 heteroatoms selected from N, O;
6-membered heterocyclic containing 1, 2 or 3 heteroatoms selected from N, O; 3-membered carbocyclic; 4-membered carbocyclic; 5-membered carbocyclic; or 6-membered carbocyclic; and each of which at each occurrence is independently optionally substituted with 1, 2, 3, 4, 5 or 6 substituents, and the said each of substituents at each occurrence is selected from deuterium, halogen, —NH₂, —CN, —OH, —NO₂, carboxyl, —C₁₋₃ alkyl, or —C₁₋₃alkoxy;
W₂ is selected from hydrogen; deuterium; —F; —CI; —NH₂; —CN; —OH; carboxyl; —C₁₋₃ alkyl; —C₁₋₃alkoxy; phenyl; naphthyl; 5-membered heteroaryl containing 1, 2 or 3 heteroatoms selected from N, O or S; 6-membered heteroaryl containing 1, 2 or 3 heteroatoms selected from N, O, or S; 7-membered heteroaryl containing 1, 2 or 3 heteroatoms selected from N, O or S; 8-membered heteroaryl containing 1, 2 or 3 heteroatoms selected from N, O or S; 9-membered heteroaryl containing 1, 2 or 3 heteroatoms selected from N, O or S; 10-membered heteroaryl containing 1, 2 or 3 heteroatoms selected from N, O, or S; 3-membered heterocyclic containing 1, 2 or 3 heteroatoms selected from N, O or S; 4-membered heterocyclic containing 1, 2 or 3 heteroatoms selected from N, O or S; 5-membered heterocyclic containing 1, 2 or 3 heteroatoms selected from N, O or S; 6-membered heterocyclic containing 1, 2 or 3 heteroatoms selected from N, O or S; 3-membered carbocyclic; 4-membered carbocyclic; 5-membered carbocyclic; or 6-membered carbocyclic; and each of which at each occurrence is independently optionally substituted with 1, 2, 3, 4 or 5 substituents, and the said each of substituents at each occurrence is selected from deuterium, halogen, —NH₂, —CN, —OH, —NO₂, carboxyl, methyl, ethyl, propyl, isopropyl, methoxy, ethoxy, propoxy or isopropoxy;
Z is selected from hydrogen, deuterium, halogen, —NH₂, —CN, —OH, carboxyl, —C₁₋₆alkyl or —C₁₋₆alkoxy.

76. A method of treating a cancer in a subject in need thereof, said method comprising administering a pharmaceutical composition to said subject,
wherein said pharmaceutical composition comprises a bromodomain and extra-terminal domain (BET) inhibitor;
said cancer has previously been determined to comprise a decreased amount or activity of p300 compared to wild type p300;
wherein said BET inhibitor comprises a compound of formula IV, 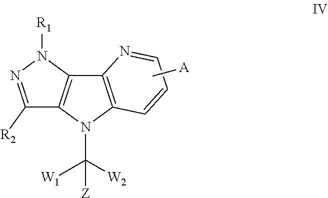
or a pharmaceutically acceptable salt thereof or a stereoisomer thereof
wherein:
R₁ is selected from hydrogen; deuterium; —C₁₋₆alkyl; or —C₃₋₈carbocyclic; and each of which at each occurrence is independently optionally substituted with 1, 2, 3, 4, 5 or 6 substituents, and the said each of substituents at each occurrence is independently selected from deuterium, halogen, OH, —CN, —C₁₋₈alkyl, —C₁₋₈alkoxy, —NH₂, —NH(C₁₋₆ alkyl), —N(C₁₋₆alkyl)₂, or —C₃₋₈carbocyclic;
R₂ is selected from hydrogen; deuterium; halogen; —OR₂₁; —NR₂₁R₂₂; —CN; —SR₂₁; —SOR₂₁; —SO₂R₂₁; —SO₂NR₂₁R₂₂; —C₁₋₈alkyl; 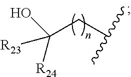
carboxyl; —COOR₂₁; —CONR₂₁R₂₂; —NR₂₁COR₂₂; —NR₂₁SO₂R₂₂; or —C₃₋₈carbocyclic; and each of which at each occurrence is independently optionally substituted with 1, 2, 3, 4, 5 or 6 substituents, and the said each of substituents at each occurrence is independently selected from deuterium, halogen, —OH, —CN, —NH₂, —C₁₋₈alkyl, —C₁₋₈alkoxy, C₃₋₈carbocyclic, or 6-membered heterocyclic containing 1, 2 or 3 heteroatoms selected from N, O;
each of R₂₁ and R₂₂ at each occurrence is independently selected from hydrogen; deuterium; —OH; NH₂; —CN; —C₁₋₈alkyl; —C₁₋₈alkoxy; —C₁₋₈alkylene-C₃₋₈carbocyclic; or —C₃₋₈carbocyclic;
each of R₂₃ and R₂₄ at each occurrence is independently selected from hydrogen, deuterium, or —C₁₋₈alkyl;
A is selected from 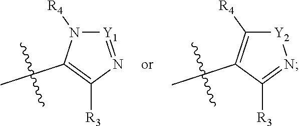
Y₁ is selected from N or CR_{Y1};
Y₂ is selected from O, S, CR_{Y1}R_{Y2} or NR_{Y2};
each of R_{Y1} and R_{Y2} at each occurrence is independently selected from hydrogen, deuterium, halogen, —OH, NH₂, —CN, —C₁₋₆alkyl or —C₁₋₆alkoxy;
each of R₃ and R₄ at each occurrence is independently selected from hydrogen, deuterium, or —C₁₋₆alkyl; and each of which at each occurrence is independently optionally substituted with 1, 2, 3, 4, 5 or 6 substituents, and the said each of substituents at each occurrence is independently selected from deuterium, halogen, —OH, —NH₂, —CN, —C₁₋₆alkyl or —C₁₋₆alkoxy;
n is selected from 0, 1, 2, 3, 4, 5 or 6;
W₁ is selected from hydrogen; deuterium; —F; —CI; —NH₂; —CN; —OH; carboxyl; —C₁₋₆ alkyl; —C₁₋₆alkoxy; —C₁₋₃alkylene-C₁₋₃alkoxy; phenyl; 5-membered heteroaryl containing 1, 2 or 3 heteroatoms selected from N, O; 6-membered heteroaryl containing 1, 2 or 3 heteroatoms selected from N, O; 3-membered heterocyclic containing 1, 2 or 3 heteroatoms selected from N, O; 4-membered heterocyclic containing 1, 2 or 3 heteroatoms selected from N, O; 5-membered heterocyclic containing 1, 2 or 3 heteroatoms selected from N, O;
6-membered heterocyclic containing 1, 2 or 3 heteroatoms selected from N, O; 3-membered carbocyclic; 4-membered carbocyclic; 5-membered carbocyclic; or 6-membered carbocyclic; and each of which at each occurrence is independently optionally substituted with 1, 2, 3, 4, 5 or 6 substituents, and the said each of substituents at each occurrence is selected from deuterium, halogen, —NH₂, —CN, —OH, —NO₂, carboxyl, —C₁₋₃ alkyl, or —C₁₋₃alkoxy;
W₂ is selected from hydrogen; deuterium; —F; —CI; —NH₂; —CN; —OH; carboxyl; —C₁₋₃ alkyl; —C₁₋₃alkoxy; phenyl; naphthyl; 5-membered heteroaryl containing 1, 2 or 3 heteroatoms selected from N, O or S; 6-membered heteroaryl containing 1, 2 or 3 heteroatoms selected from N, O, or S; 7-membered heteroaryl containing 1, 2 or 3 heteroatoms selected from N, O or S; 8-membered heteroaryl containing 1, 2 or 3 heteroatoms selected from N, O or S; 9-membered heteroaryl containing 1, 2 or 3 heteroatoms selected from N, O or S; 10-membered heteroaryl containing 1, 2 or 3 heteroatoms selected from N, O, or S; 3-membered heterocyclic containing 1, 2 or 3 heteroatoms selected from N, O or S; 4-membered heterocyclic containing 1, 2 or 3 heteroatoms selected from N, O or S; 5-membered heterocyclic containing 1, 2 or 3 heteroatoms selected from N, O or S; 6-membered heterocyclic containing 1, 2 or 3 heteroatoms selected from N, O or S; 3-membered carbocyclic; 4-membered carbocyclic; 5-membered carbocyclic; or 6-membered carbocyclic; and each of which at each occurrence is independently optionally substituted with 1, 2, 3, 4 or 5 substituents, and the said each of substituents at each occurrence is selected from deuterium, halogen, —NH₂, —CN, —OH, —NO₂, carboxyl, methyl, ethyl, propyl, isopropyl, methoxy, ethoxy, propoxy or isopropoxy;
Z is selected from hydrogen, deuterium, halogen, —NH₂, —CN, —OH, carboxyl, —C₁₋₆alkyl or —C₁₋₆alkoxy.

77. The method of claim 75, wherein said loss of function is caused by genetic mutation.

78. The method of claim 75 or 76, wherein said BET inhibitor comprises Compound 5: 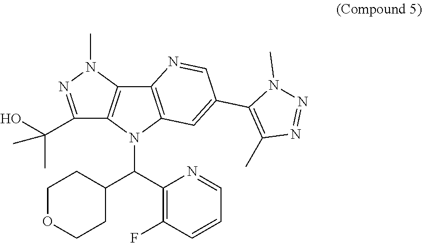 [SMILES: Cc1nnn(C)c1-c1cnc2c3c(c(C(C)(C)O)nn3C)n(C(c3ncccc3F)C3CCOCC3)c2c1]
or a pharmaceutically acceptable salt, a tautomer, a solvate, or a deuterated analog thereof.

79. The method of claim 75 or 76, wherein said BET inhibitor comprises Compound 6: 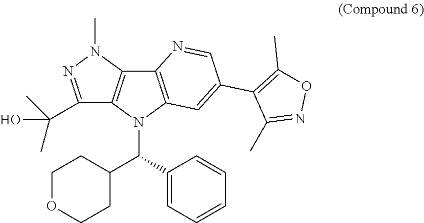 [SMILES: Cc1noc(C)c1-c1cnc2c3c(c(C(C)(C)O)nn3C)n([C@H](c3ccccc3)C3CCOCC3)c2c1]
or a pharmaceutically acceptable salt, a tautomer, a solvate, or a deuterated analog thereof.

80. The method of claim 75 or 76, wherein said BET inhibitor comprises Compound 7: 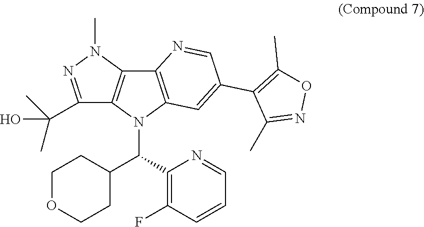 [SMILES: Cc1noc(C)c1-c1cnc2c3c(c(C(C)(C)O)nn3C)n([C@H](c3ncccc3F)C3CCOCC3)c2c1]
or a pharmaceutically acceptable salt, a tautomer, a solvate, or a deuterated analog thereof.

81. The method of claim 75 or 76, wherein said BET inhibitor comprises Compound 8: 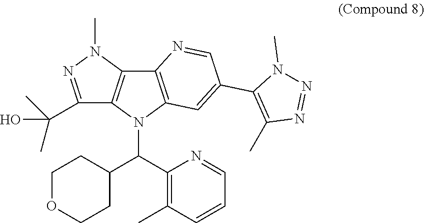 [SMILES: Cc1cccnc1C(C1CCOCC1)n1c2cc(-c3c(C)nnn3C)cnc2c2c1c(C(C)(C)O)nn2C]
or a pharmaceutically acceptable salt, a tautomer, a solvate, or a deuterated analog thereof.

82. The method of claim 75 or 76, wherein said cancer comprises bladder cancer, lung cancer, gynecologic cancer, adrenocortical carcinoma, bone cancer, central nervous system (CNS) cancer, pancreatic cancer, gastrointestinal cancer, head and neck cancer, skin cancer, mesothelioma, nerve sheath tumor, lymphoma, renal cell carcinoma, salivary gland cancer, non-melanoma skin cancer, or a combination thereof.

83. The method of claim 82, wherein said cancer comprises lung cancer.

84. The method of claim 83, wherein said lung cancer comprises non-small cell lung cancer.

85. The method of claim 84, wherein said non-small cell lung cancer comprises squamous cell carcinoma, large cell carcinoma, adenocarcinoma, or a combination thereof.

86. The method of claim 85, wherein said non-small cell lung cancer comprises squamous cell carcinoma.

87. The method of claim 85, wherein said non-small cell lung cancer comprises large cell carcinoma.

88. The method of claim 85, wherein said non-small cell lung cancer comprises adenocarcinoma.

89. The method of claim 84, wherein said lung cancer comprises small cell lung cancer.

90. The method of claim 82, wherein said cancer comprises bladder cancer.

91. The method of claim 90, wherein said bladder cancer comprises a carcinoma.

92. The method of claim 91, wherein carcinoma comprises urothelial carcinoma.

93. The method of claim 82, wherein said cancer comprises pancreatic cancer.

94. The method of claim 82, wherein said skin cancer comprises melanoma.

95. The method of claim 82, wherein said CNS cancer comprises a glioma.

96. The method of claim 82, wherein said CNS cancer comprises a neuroepithelial tumor.

97. The method of claim 82, wherein said lymphoma comprises Non-Hodgkin lymphoma.

98. The method of claim 82, wherein said lymphoma comprises diffuse large B cell lymphoma.

99. The method of claim 82, wherein said lymphoma comprises follicular lymphoma.

100. The method of claim 82, wherein said lymphoma comprises marginal zone lymphoma.

101. The method of claim 82, wherein said lymphoma comprises mature B-cell neoplasm.

102. The method of claim 82, wherein said gastrointestinal cancer comprises a gastrointestinal stromal tumor, esophagogastric cancer, gastrointestinal neuroendocrine tumor, small bowel cancer, anal cancer, colon cancer, or a combination thereof.

103. The method of claim 82, wherein said gastrointestinal cancer comprises a gastrointestinal stromal tumor.

104. The method of claim 82, wherein said gastrointestinal cancer comprises an esophagogastric cancer.

105. The method of claim 82, wherein said gastrointestinal cancer comprises a gastrointestinal neuroendocrine tumor.

106. The method of claim 82, wherein said gastrointestinal cancer comprises a small bowel cancer.

107. The method of claim 82, wherein said gastrointestinal cancer comprises an anal cancer.

108. The method of claim 82, wherein said gastrointestinal cancer comprises colon cancer.

109. The method of claim 108, wherein said colon cancer comprises colorectal cancer.

110. The method of claim 82, wherein said gynecologic cancer comprises a cervical cancer.

111. The method of claim 82, wherein said gynecologic cancer comprises an ovarian cancer.

112. The method of claim 82, wherein said gynecologic cancer comprises a sex cord stromal tumor.

113. The method of claim 82, wherein said gynecologic cancer comprises a vaginal cancer.

114. The method of claim 82, wherein said cancer comprises uterine cancer.

115. The method of claim 114, wherein said uterine cancer comprises endometrial cancer or uterine sarcoma.

116. The method of claim 115, wherein said uterine cancer comprises endometrial cancer.

117. The method of claim 116, wherein said endometrial cancer comprises uterine corpus endometrial carcinoma.

118. The method of claim 114, wherein said uterine cancer comprises uterine sarcoma.

119. The method of claim 118, wherein said uterine sarcoma comprises uterine carcinosarcoma.

120. The method of claim 75, wherein said subject has a NUT midline cancer.

121. The method of claim 75, wherein said subject has a castration resistant prostate cancer.

122. The method of claim 75, wherein said subject has a solid cancer or a liquid cancer.

123. The method of claim 75, wherein said subject has a liquid cancer selected from myelofibrosis.

124. The method of claim 75, wherein said subject has a liquid cancer selected from myeloma and leukemia.

125. The method of claim 75, wherein said subject has a liquid cancer selected from leukemia.

126. The method of claim 124, wherein the leukemia is selected from acute myeloid leukemia (AML) and acute lymphocytic leukemia (ALL).

127. The method of claim 75, wherein said loss of function or deletion is for an EP300 gene.

128. The method of claim 75 or 76, wherein:
R₁ is selected from hydrogen; deuterium; —C₁₋₆alkyl; or —C₃₋₈carbocyclic; and each of which at each occurrence is independently optionally substituted with 1, 2, 3, 4, 5 or 6 substituents, and the said each of substituents at each occurrence is independently selected from deuterium, halogen, OH, —CN, —C₁₋₈alkyl, or —C₁₋₈alkoxy;
R₂ is selected from hydrogen; deuterium; halogen; —C₁₋₈alkyl; 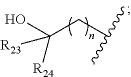
carboxyl; —COOR₂₁; or —CONR₂₁R₂₂; and each of which at each occurrence is independently optionally substituted with 1, 2, 3, 4, 5 or 6 substituents, and the said each of substituents at each occurrence is independently selected from deuterium, halogen, —OH, —CN, —NH₂, —C₁₋₈alkyl, —C₁₋₈alkoxy, C₃₋₈carbocyclic, or 6-membered heterocyclic containing 1, 2 or 3 heteroatoms selected from N, O;
each of R₂₁ and R₂₂ at each occurrence is independently selected from hydrogen; deuterium; —OH; NH₂; —CN; —C₁₋₈alkyl; or —C₃₋₈carbocyclic;
each of R₂₃ and R₂₄ at each occurrence is independently selected from hydrogen, deuterium, or —C₁₋₈alkyl;
A is selected from 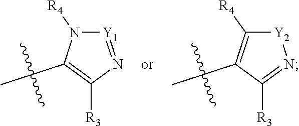
Y₁ is selected from N or CR_{Y1};
Y₂ is selected from O, S, CR_{Y1}R_{Y2} or NR_{Y2};
each of R_{Y1} and R_{Y2} at each occurrence is independently selected from hydrogen, deuterium, halogen, —OH, NH₂, —CN, —C₁₋₆alkyl or —C₁₋₆alkoxy;
each of R₃ and R₄ at each occurrence is independently selected from hydrogen, deuterium, or —C₁₋₆alkyl; and each of which at each occurrence is independently optionally substituted with 1, 2, 3, 4, 5 or 6 substituents, and the said each of substituents at each occurrence is independently selected from deuterium, halogen, —OH, —NH₂, —CN, —C₁₋₆alkyl or —C₁₋₆alkoxy;
n is selected from 0, 1 or 2;
W₁ is selected from hydrogen; deuterium; —F; —CI; —NH₂; —CN; —OH; carboxyl; —C₁₋₆ alkyl; —C₁₋₆alkoxy; 6-membered heterocyclic containing 1, 2 or 3 heteroatoms selected from N, O; and each of which at each occurrence is independently optionally substituted with 1, 2, 3, 4, 5 or 6 substituents, and the said each of substituents at each occurrence is selected from deuterium, halogen, —NH₂, —CN, —OH, —NO₂, carboxyl, —C₁₋₃alkyl, or —C₁₋₃ alkoxy;
W₂ is selected from hydrogen; deuterium; —F; —CI; —NH₂; —CN; —OH; carboxyl; —C₁₋₃ alkyl; —C₁₋₃alkoxy; phenyl; naphthyl; 5-membered heteroaryl containing 1, 2 or 3 heteroatoms selected from N, O or S; 6-membered heteroaryl containing 1, 2 or 3 heteroatoms selected from N, O, or S; and each of which at each occurrence is independently optionally substituted with 1, 2, 3, 4 or 5 substituents, and the said each of substituents at each occurrence is selected from deuterium, halogen, —NH₂, —CN, —OH, —NO₂, carboxyl, methyl, ethyl, propyl, isopropyl, methoxy, ethoxy, propoxy or isopropoxy;
Z is selected from hydrogen, deuterium, halogen, —NH₂, —CN, —OH, or —C₁₋₆alkoxy.

129. The method of claim 75 or 76, wherein said compound of formula IV is selected from:
(S)-2-(6-(3,5-dimethylisoxazol-4-yl)-1-methyl-4-(phenyl(tetrahydro-2H-pyran-4-yl)methyl)-1,4-dihydropyrazolo[3′,4′:4,5]pyrrolo[3,2-b]pyridin-3-yl)propan-2-ol;
(S)-2-(6-(3,5-dimethylisoxazol-4-yl)-4-((3-fluoropyridin-2-yl)(tetrahydro-2H-pyran-4-yl)methyl)-1-methyl-1,4-dihydropyrazolo[3′,4′:4,5]pyrrolo[3,2-b]pyridin-3-yl)propan-2-ol;
2-(6-(1,4-Dimethyl-1H-1,2,3-triazol-5-yl)-1-methyl-4-(4,4,4-trifluoro-1-(3-fluoropyridin-2-yl)butyl)-1,4-dihydropyrazolo[3′,4′:4,5]pyrrolo[3,2-b]pyridine-3-yl)propan-2-ol;
2-(6-(1,4-Dimethyl-1H-1,2,3-triazol-5-yl)-1-methyl-4-((3-methylpyridin-2-yl)(tetrahydro-2H-pyran-4-yl)methyl)-1,4-dihydropyrazolo[3′,4′:4,5]pyrrolo[3,2-b]pyridine-3-yl)propan-2-ol;
(S)-2-(6-(1,4-dimethyl-1H-1,2,3-triazol-5-yl)-1-methyl-4-(phenyl(tetrahydro-2H-pyran-4-yl)methyl)-1,4-dihydropyrazolo[3′,4′:4,5]pyrrolo[3,2-b]pyridine-3-yl)propan-2-ol;
(S)-2-(6-(1,4-dimethyl-1H-1,2,3-triazol-5-yl)-4-((3-fluoropyridin-2-yl)(tetrahydro-2H-pyran-4-yl)methyl)-1-methyl-1,4-dihydropyrazolo[3′,4′:4,5]pyrrolo[3,2-b]pyridin-3-yl)propan-2-ol;
2-(6-(3,5-dimethylisoxazol-4-yl)-1-methyl-4-((tetrahydro-2H-pyran-4-yl)(o-tolyl)methyl)-1,4-dihydropyrazolo[3′,4′:4,5]pyrrolo[3,2-b]pyridin-3-yl)propan-2-ol;
(S)-2-(6-(1,4-Dimethyl-1H-1,2,3-triazol-5-yl)-4-((3-fluoropyridin-2-yl)(tetrahydro-2H-pyran-4-yl)methyl)-1,4-dihydropyrazolo[3′,4′:4,5]pyrrolo[3,2-b]pyridin-3-yl)propan-2-ol;
6-(1,4-Dimethyl-1H-1,2,3-triazol-5-yl)-1-methyl-4-(phenyl(tetrahydro-2H-pyran-4-yl)methyl)-1,4-dihydropyrazolo[3′,4′:4,5]pyrrolo[3,2-b]pyridine-3-carboxamide;
2-(6-(1,4-Dimethyl-1H-1,2,3-triazol-5-yl)-1-methyl-4-(phenyl(tetrahydro-2H-pyran-4-yl)methyl)-1,4-dihydropyrazolo[3′,4′:4,5]pyrrolo[3,2-b]pyridine-3-yl)propan-2-amine;
2-(4-((3-Fluoropyridin-2-yl)(tetrahydro-2H-pyran-4-yl)methyl)-1-methyl-6-(1-methyl-4-(methyl-d3)-1H-1,2,3-triazol-5-yl)-1,4-dihydropyrazolo[3′,4′:4,5]pyrrolo[3,2-b]pyridine-3-yl)propan-2-ol;
2-(6-(1,4-Dimethyl-1H-1,2,3-triazol-5-yl)-4-((3-fluoropyridin-4-yl)(tetrahydro-2H-pyran-4-yl)methyl)-1-methyl-1,4-dihydropyrazolo[3′,4′:4,5]pyrrolo[3,2-b]pyridine-3-yl)propan-2-ol;
2-(6-(1,4-Dimethyl-1H-1,2,3-triazol-5-yl)-4-((3-methoxypyridin-2-yl)(tetrahydro-2H-pyran-4-yl)methyl)-1-methyl-1,4-dihydropyrazolo[3′,4′:4,5]pyrrolo[3,2-b]pyridin-3-yl)propan-2-ol; and
4-((6-(1,4-dimethyl-1H-1,2,3-triazol-5-yl)-1-methyl-4-(phenyl(tetrahydro-2H-pyran-4-yl)methyl)-1,4-dihydropyrazolo[3′,4′:4,5]pyrrolo[3,2-b]pyridin-3-yl)methyl)morpholine.
